SEQUENCE LISTING (24 sequences)

SEQ ID NO: 1 - DNA, 43 bp, synthetic construct
  source 1..43: mol_type = other DNA
ggcataactagnnnngctagttcgcatacgnnnngcatcgtac

SEQ ID NO: 2 - DNA, 10 bp, synthetic construct
  source 1..10: mol_type = other DNA
  modified_base 1: mod_base = OTHER; note = 5'-Phosphate modified nucleotide
cgtatgcgaa

SEQ ID NO: 3 - DNA, 10 bp, synthetic construct
  source 1..10: mol_type = other DNA
  modified_base 1: mod_base = OTHER; note = 5'-Phosphate modified nucleotide
ctagttatgc

SEQ ID NO: 4 - DNA, 10 bp, synthetic construct
  source 1..10: mol_type = other DNA
  misc_difference 1: note = Nucleotide attached to Dye1 which may be selected from any fluorescent dye and is spectrally distinct from Dyes 2-4
gtacgatgca

SEQ ID NO: 5 - DNA, 10 bp, synthetic construct
  source 1..10: mol_type = other DNA
  misc_difference 1: note = Nucleotide attached to Dye2 which may be selected from any fluorescent dye and is spectrally distinct from Dyes 1 and 3-4
gtacgatgct

SEQ ID NO: 6 - DNA, 10 bp, synthetic construct
  source 1..10: mol_type = other DNA
  misc_difference 1: note = Nucleotide attached to Dye3 which may be selected from any fluorescent dye and is spectrally distinct from Dyes 1-2, and 4
gtacgatgcc

SEQ ID NO: 7 - DNA, 10 bp, synthetic construct
  source 1..10: mol_type = other DNA
  misc_difference 1: note = Nucleotide attached to Dye4 which may be selected from any fluorescent dye and is spectrally distinct from Dyes 1-3
gtacgatgcg

SEQ ID NO: 8 - DNA, 10 bp, synthetic construct
  source 1..10: mol_type = other DNA
  modified_base 1: mod_base = OTHER; note = 5'-Phosphate modified nucleotide
  misc_difference 10: note = Nucleotide attached to Dye1 which may be selected from any fluorescent dye and is spectrally distinct from Dyes 2-4
acgtatgcga

SEQ ID NO: 9 - DNA, 10 bp, synthetic construct
  source 1..10: mol_type = other DNA
  modified_base 1: mod_base = OTHER; note = 5'-Phosphate modified nucleotide
  misc_difference 10: note = Nucleotide attached to Dye2 which may be selected from any fluorescent dye and is spectrally distinct from Dyes 1 and 3-4
tcgtatgcga

SEQ ID NO: 10 - DNA, 10 bp, synthetic construct
  source 1..10: mol_type = other DNA
  modified_base 1: mod_base = OTHER; note = 5'-Phosphate modified nucleotide
  misc_difference 10: note = Nucleotide attached to Dye3 which may be selected from any fluorescent dye and is spectrally distinct from Dyes 1-2, and 4
ccgtatgcga

SEQ ID NO: 11 - DNA, 10 bp, synthetic construct
  source 1..10: mol_type = other DNA
  modified_base 1: mod_base = OTHER; note = 5'-Phosphate modified nucleotide
  misc_difference 10: note = Nucleotide attached to Dye4 which may be selected from any fluorescent dye and is spectrally distinct from Dyes 1-3
gcgtatgcga

SEQ ID NO: 12 - DNA, 10 bp, synthetic construct
  source 1..10: mol_type = other DNA
gtacgatgca

SEQ ID NO: 13 - DNA, 10 bp, synthetic construct
  source 1..10: mol_type = other DNA
gtacgatgct

SEQ ID NO: 14 - DNA, 10 bp, synthetic construct
  source 1..10: mol_type = other DNA
gtacgatgcc

SEQ ID NO: 15 - DNA, 30 bp, synthetic construct
  source 1..30: mol_type = other DNA
  misc_difference 1..20: note = This region may encompass 5-20 nucleotides
nnnnnnnnnnnnnnnnnnnngtacgatgcg

SEQ ID NO: 16 - DNA, 10 bp, synthetic construct
  source 1..10: mol_type = other DNA
gtacgatgcg

SEQ ID NO: 17 - DNA, 10 bp, synthetic construct
  source 1..10: mol_type = other DNA
  modified_base 1: mod_base = OTHER; note = 5'-Phosphate modified nucleotide
  misc_difference 10: note = Nucleotide attached to Dye4 which may be selected from any fluorescent dye and is spectrally distinct from Dyes 1-3
gcgtatgcga

SEQ ID NO: 18 - DNA, 10 bp, synthetic construct
  source 1..10: mol_type = other DNA
  misc_difference 1: note = Nucleotide attached to Dye1 which may be selected from any fluorescent dye and is spectrally distinct from Dyes 2-4
  misc_difference 7: note = Nucleotide attached to Dye1 which may be selected from any fluorescent dye and is spectrally distinct from Dyes 2-4
gtacgatgca

SEQ ID NO: 19 - DNA, 10 bp, synthetic construct
  source 1..10: mol_type = other DNA
  misc_difference 1: note = Nucleotide attached to Dye2 which may be selected from any fluorescent dye and is spectrally distinct from Dyes 1 and 3-4
  misc_difference 7: note = Nucleotide attached to Dye2 which may be selected from any fluorescent dye and is spectrally distinct from Dyes 1 and 3-4
gtacgatgct

SEQ ID NO: 20 - DNA, 10 bp, synthetic construct
  source 1..10: mol_type = other DNA
  misc_difference 1: note = Nucleotide attached to Dye3 which may be selected from any fluorescent dye and is spectrally distinct from Dyes 1-2, and 4
  misc_difference 7: note = Nucleotide attached to Dye3 which may be selected from any fluorescent dye and is spectrally distinct from Dyes 1-2, and 4
gtacgatgcc

SEQ ID NO: 21 - DNA, 10 bp, synthetic construct
  source 1..10: mol_type = other DNA
  misc_difference 1: note = Nucleotide attached to Dye4 which may be selected from any fluorescent dye and is spectrally distinct from Dyes 1-3
  misc_difference 7: note = Nucleotide attached to Dye4 which may be selected from any fluorescent dye and is spectrally distinct from Dyes 1-3
gtacgatgcg

SEQ ID NO: 22 - DNA, 30 bp, synthetic construct
  source 1..30: mol_type = other DNA
  misc_difference 1..20: note = This region may encompass 5-20 nucleotides
nnnnnnnnnnnnnnnnnnnngtacgatgca

SEQ ID NO: 23 - DNA, 30 bp, synthetic construct
  source 1..30: mol_type = other DNA
  misc_difference 1..20: note = This region may encompass 5-20 nucleotides
nnnnnnnnnnnnnnnnnnnngtacgatgct

SEQ ID NO: 24 - DNA, 30 bp, synthetic construct
  source 1..30: mol_type = other DNA
  misc_difference 1..20: note = This region may encompass 5-20 nucleotides
nnnnnnnnnnnnnnnnnnnngtacgatgcc

(19) United States
(12) Patent Application Publication
(10) Pub. No.: US 2026/0125743 A1
(43) Pub. Date: May 7, 2026

(54) COMPOSITIONS AND METHODS FOR TARGET DETECTION

(71) Applicant: Stellaromics, Inc., Boston, MA (US)

(72) Inventors: Ye FU, Boston, MA (US); Mark PRATT, Bozeman, MT (US)

(21) Appl. No.: 19/344,362

(22) Filed: Sep. 29, 2025

Related U.S. Application Data
(63) Continuation of application No. PCT/US2025/030656, filed on May 22, 2025.
(60) Provisional application No. 63/651,318, filed on May 23, 2024.

(51) Int. Cl. C12Q 1/6809

(52) U.S. Cl. CPC C12Q 1/6809

(57) ABSTRACT

Provided herein are methods for identifying subsequences in a sample. The methods may comprise contacting an analyte with a binding moiety, bringing the analyte in contact with a first detection probe and second detection probe, wherein the first detection probe and second detection probe are adjacent and different from one another, detecting the corresponding detection moieties, and using the identified subsequences to identify the analyte.

### CROSS-REFERENCE

This application is a continuation of Int'l Application No. PCT/US2025/030656, filed May 22, 2025, which claims priority to U.S. Provisional Patent Application No. 63/651,318, filed May 23, 2024, each of which is entirely incorporated herein by reference.

### SEQUENCE LISTING

The instant application contains a Sequence Listing which has been submitted electronically in XML format and is hereby incorporated by reference in its entirety. Said XML copy, created on Sep. 29, 2025, is named 68135-718.301_SL.xml and is 35,803_bytes in size.

### BACKGROUND

Targets can be detected in situ within a biological sample. Multiple methods of detection can be used.

### SUMMARY

In an aspect, the present disclosure provides methods for identifying an analyte in a sample, comprising: (a) contacting the sample with a binding moiety, wherein the binding moiety comprises (i) a probe that couples to the analyte and (ii) a sequence, wherein the sequence comprises a plurality of subsequences; (b) bringing the sample in contact with a first detection probe and a second detection probe, wherein: (i) the first detection probe couples to a first subsequence of the sequence or derivative thereof, and wherein the first detection probe comprises a first detection moiety; (ii) the second detection probe couples to a second subsequence of the sequence or derivative thereof, and wherein the second detection probe comprises a second detection moiety; and (iii) the first subsequence and the second subsequence are adjacent to each other; (c) detecting the first detection moiety and the second detection moiety to identify at least the first subsequence and the second subsequence; and (d) using at least the first subsequence and the second subsequence identified in (c) to identify the analyte.

In some embodiments, the first detection moiety and the second detection moiety are different. In some embodiments, the first detection moiety and the second detection moiety are the same. In some embodiments, the sample is a tissue. In some embodiments, the tissue is a fresh-frozen tissue. In some embodiments, the tissue is a formalin-fixed paraffin embedded tissue. In some embodiments, the sample comprises a cell. In some embodiments, the sample is embedded in a hydrogel. In some embodiments, the analyte comprises a protein or a polypeptide. In some embodiments, the analyte comprises a nucleic acid. In some embodiments, the nucleic acid is deoxyribonucleic acid. In some embodiments, the nucleic acid is ribonucleic acid. In some embodiments, the probe that couples to the analyte comprises an antibody or antibody fragment. In some embodiments, the probe that couples to the analyte comprises a nucleic acid. In some embodiments, the probe that couples to the analyte comprises an aptamer. In some embodiments, a subsequence of the plurality of subsequences comprises a nucleic acid. In some embodiments, the nucleic acid comprises one or more nucleotides. In some embodiments, the nucleic acid comprises two or more nucleotides. In some embodiments, the nucleic acid comprises three or more nucleotides. In some embodiments, the plurality of subsequences comprises subsequences of the same length. In some embodiments, the plurality of subsequences comprises subsequences of different lengths. In some embodiments, the first detection probe comprises a nucleic acid. In some embodiments, the second detection probe comprises a nucleic acid. In some embodiments, the first subsequence is a nucleic acid. In some embodiments, the nucleic acid is at least one nucleotide in length. In some embodiments, the nucleic acid is at least two nucleotides in length. In some embodiments, the nucleic acid is at least three nucleotides in length. In some embodiments, the second subsequence is a nucleic acid. In some embodiments, the nucleic acid is at least one nucleotide in length. In some embodiments, the nucleic acid is at least two nucleotides in length. In some embodiments, the nucleic acid is at least three nucleotides in length. In some embodiments, the first detection moiety comprises a fluorescent dye. In some embodiments, the first detection moiety comprises a linker. In some embodiments, the first detection moiety is coupled to the nucleic acid of the first detection probe via the linker. In some embodiments, the first detection moiety is coupled to a 5′ end of the nucleic acid of the first detection probe. In some embodiments, the first detection moiety is coupled to a 3′ end of the nucleic acid of the first detection probe. In some embodiments, the second detection moiety comprises a fluorescent dye. In some embodiments, the second detection moiety comprises a linker. In some embodiments, the second detection moiety is coupled to the nucleic acid of the second detection probe via the linker. In some embodiments, the second detection moiety is coupled to a 3′ end of the nucleic acid of the second detection probe. In some embodiments, the second detection moiety is coupled to a 5′ end of the nucleic acid of the second detection probe. In some embodiments, (c) comprises imaging the analyte. In some embodiments, the imaging comprises using a microscope. In some embodiments, the method further comprises amplifying the sequence in (a) to generate an amplified sequence, wherein the amplified sequence comprises multiple copies of the sequence or derivative thereof. In some embodiments, the method further comprises ligating the first detection probe and the second detection probe in (b). In some embodiments, the ligating comprises using a ligase. In some embodiments, the ligase is a T4 ligase. In some embodiments, the ligating comprises reacting a first reactive chemical moiety and a second reactive chemical moiety, wherein the first detection probe comprises the first reactive chemical moiety and the second detection probe comprises the second reactive chemical moiety. In some embodiments, the first reactive chemical moiety is selected from the group consisting of tetrazine, alkyne, azide, trans-cyclooctene, maleimide, N-hydroxysuccinimide ester, amine, carboxylic acid, hydroxyl, cyclopropenone, thiol, and norbornene. In some embodiments, the second reactive chemical moiety is selected from the group consisting of tetrazine, alkyne, azide, trans-cyclooctene, maleimide, N-hydroxysuccinimide ester, amine, carboxylic acid, hydroxyl, cyclopropenone, thiol, and norbornene. In some embodiments, (c) further comprises detecting a ratio of the first detection moiety and the second detection moiety. In some embodiments, the method further comprises using the ratio to identify the analyte. In some embodiments, the method further comprises detecting a third subsequence of the sequence and a fourth subsequence of the sequence using a third detection probe and a fourth detection probe to identify the third subsequence and the fourth subsequence. In some embodiments, the third detection probe comprises a third detection moiety and wherein the fourth detection probe comprises a fourth detection moiety. In some embodiments, the third detection moiety and the fourth detection moiety are the same. In some embodiments, the third detection moiety and the fourth detection moiety are different. In some embodiments, the third detection moiety is the same as either the first detection moiety or the second detection moiety. In some embodiments, the method further comprises using the third subsequence and the fourth subsequence in (d) to identify the analyte.

In another aspect, the present disclosure provides methods for identifying an analyte in a sample, comprising: (a) contacting the sample with a binding moiety, wherein the binding moiety comprises (i) a probe that couples to the analyte and (ii) a sequence, wherein the sequence comprises a plurality of subsequences; (b) bringing the sample in contact with a first detection probe and a second detection probe, wherein: (i) the first detection probe couples to a first subsequence of the sequence or derivative thereof, and wherein the first detection probe comprises a first detection moiety; (ii) the second detection probe couples to a second subsequence of the sequence or derivative thereof, and wherein the second detection probe comprises a second detection moiety; and (iii) the first subsequence is covalently linked to the second subsequence; (c) detecting the first detection moiety and the second detection moiety to identify at least the first subsequence and the second subsequence; and (d) using at least the first subsequence and the second subsequence identified in (c) to identify the analyte.

In some embodiments, the first detection moiety and the second detection moiety are different. In some embodiments, the first detection moiety and the second detection moiety are the same. In some embodiments, the sample is a tissue. In some embodiments, the tissue is a fresh-frozen tissue. In some embodiments, the tissue is a formalin-fixed paraffin embedded tissue. In some embodiments, the sample comprises a cell. In some embodiments, the sample is embedded in a hydrogel. In some embodiments, the analyte comprises a protein or a polypeptide. In some embodiments, the analyte comprises a nucleic acid. In some embodiments, the nucleic acid is deoxyribonucleic acid. In some embodiments, the nucleic acid is ribonucleic acid. In some embodiments, the probe that couples to the analyte comprises an antibody or antibody fragment. In some embodiments, the probe that couples to the analyte comprises a nucleic acid. In some embodiments, the probe that couples to the analyte comprises an aptamer. In some embodiments, a subsequence of the plurality of subsequences comprises a nucleic acid. In some embodiments, the nucleic acid comprises one or more nucleotides. In some embodiments, the nucleic acid comprises two or more nucleotides. In some embodiments, the nucleic acid comprises three or more nucleotides. In some embodiments, the plurality of subsequences comprises subsequences of the same length. In some embodiments, the plurality of subsequences comprises subsequences of different lengths. In some embodiments, the first detection probe comprises a nucleic acid. In some embodiments, the second detection probe comprises a nucleic acid. In some embodiments, the first subsequence is a nucleic acid. In some embodiments, the nucleic acid is at least one nucleotide in length. In some embodiments, the nucleic acid is at least two nucleotides in length. In some embodiments, the nucleic acid is at least three nucleotides in length. In some embodiments, the second subsequence is a nucleic acid. In some embodiments, the nucleic acid is at least one nucleotide in length. In some embodiments, the nucleic acid is at least two nucleotides in length. In some embodiments, the nucleic acid is at least three nucleotides in length. In some embodiments, the first detection moiety comprises a fluorescent dye. In some embodiments, the first detection moiety comprises a linker. In some embodiments, the first detection moiety is coupled to the nucleic acid of the first detection probe via the linker. In some embodiments, the first detection moiety is coupled to a 5′ end of the nucleic acid of the first detection probe. In some embodiments, the first detection moiety is coupled to a 3′ end of the nucleic acid of the first detection probe. In some embodiments, the second detection moiety comprises a fluorescent dye. In some embodiments, the second detection moiety comprises a linker. In some embodiments, the second detection moiety is coupled to the nucleic acid of the second detection probe via the linker. In some embodiments, the second detection moiety is coupled to a 3′ end of the nucleic acid of the second detection probe. In some embodiments, the second detection moiety is coupled to a 5′ end of the nucleic acid of the second detection probe. In some embodiments, (c) comprises imaging the analyte. In some embodiments, the imaging comprises using a microscope. In some embodiments, the method further comprises amplifying the sequence in (a) to generate an amplified sequence, wherein the amplified sequence comprises multiple copies of the sequence or derivative thereof. In some embodiments, the method further comprises ligating the first detection probe and the second detection probe in (b). In some embodiments, the ligating comprises using a ligase. In some embodiments, the ligase is a T4 ligase. In some embodiments, the ligating comprises reacting a first reactive chemical moiety and a second reactive chemical moiety, wherein the first detection probe comprises the first reactive chemical moiety and the second detection probe comprises the second reactive chemical moiety. In some embodiments, the first reactive chemical moiety is selected from the group consisting of tetrazine, alkyne, azide, trans-cyclooctene, maleimide, N-hydroxysuccinimide ester, amine, carboxylic acid, hydroxyl, cyclopropenone, thiol, and norbornene. In some embodiments, the second reactive chemical moiety is selected from the group consisting of tetrazine, alkyne, azide, trans-cyclooctene, maleimide, N-hydroxysuccinimide ester, amine, carboxylic acid, hydroxyl, cyclopropenone, thiol, and norbornene. In some embodiments, (c) further comprises detecting a ratio of the first detection moiety and the second detection moiety. In some embodiments, the method further comprises using the ratio to identify the analyte. In some embodiments, the method further comprises detecting a third subsequence of the sequence and a fourth subsequence of the sequence using a third detection probe and a fourth detection probe to identify the third subsequence and the fourth subsequence. In some embodiments, the third detection probe comprises a third detection moiety and wherein the fourth detection probe comprises a fourth detection moiety. In some embodiments, the third detection moiety and the fourth detection moiety are the same. In some embodiments, the third detection moiety and the fourth detection moiety are different. In some embodiments, the third detection moiety is the same as either the first detection moiety or the second detection moiety. In some embodiments, the method further comprises using the third subsequence and the fourth subsequence in (d) to identify the analyte.

In another aspect, the present disclosure provides methods comprising: (a) providing said sample, wherein said sample comprises one or more analytes and wherein said one or more analytes comprises a nucleic acid; (b) contacting said analyte with a binding moiety and one or more detection probes, wherein said binding moiety comprises a sequence; and (c) detecting at least a portion of said sequence or a derivative thereof with greater than 70% accuracy, wherein said at least a portion of said sequence or a derivative thereof is detected when said two or more detection probes of the one or more detection probes are adjacent to one another.

In some embodiments, the two or more detection probes are at most 15 nucleotides in length. In some embodiments, the two or more detection probes are at most 10 nucleotides in length. In some embodiments, the nuclei acid comprises a genetic aberration. In some embodiments, the genetic aberration comprises a single nucleotide polymorphism. In some embodiments, the genetic aberration comprises a deletion. In some embodiments, the genetic aberration comprises an insertion. In some embodiments, the genetic aberration comprises a copy number variation.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows the decoding capacity for number of n states/round for k rounds without any temporal information for sequencing.

FIG. 2 shows dinucleotide combinations for a 10-state decoding scheme.

FIG. 3 shows dinucleotide combinations for a 16-state decoding scheme.

FIG. 4 shows an example of dinucleotide combinations for a 15-state decoding scheme without pre-arranged temporal information.

FIG. 5 shows image data dinucleotide combinations associated with messenger ribonucleic acid (mRNA) transcripts of a mouse brain cell using a dual-probe detection scheme.

FIG. 6 shows image data associated with mRNA transcripts of a mouse brain cell using a dual-probe detection scheme.

FIG. 7 shows a computer system that is programmed or otherwise configured to implement methods provided herein.

FIG. 8 shows a table with a detection scheme for nine genes.

FIG. 9 shows decoding probes across four rounds of detection.

FIG. 10 shows a table with the laser profiles associated with different barcodes

FIG. 11 shows images of a mouse brain tissue and detection of the ActB ribonucleic acid (RNA) signal across four rounds of detection.

FIG. 12 shows images of a mouse brain tissue and detection of the MBP RNA signal across four rounds of detection.

FIG. 13 shows images of a mouse brain tissue and detection of the Slc17a7 RNA signal across four rounds of detection.

FIG. 14 shows a process for identifying subsequences in a sample.

FIG. 15 shows a process for identifying subsequences in a sample.

FIG. 16 shows a schematic of an analyte coupled to a binding moiety.

FIG. 17 shows a process for identifying an analyte with rolling circle amplification.

FIG. 18 shows a process for identifying an analyte.

FIG. 19 shows a process for identifying an analyte using an antibody probe.

FIG. 20 shows a process for using a binder to couple to a sequence of a binding moiety.

FIG. 21 shows a process for performing two rounds of detection using detection probes.

FIG. 22 shows a process for identifying an analyte.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Provided herein are methods and/or compositions useful for the detection and/or identification of one or more analytes (e.g. targets) within a biological sample. The detection of the one or more analytes within a biological sample may comprise identifying one or more subsequence associated with the one or more analytes within the biological sample.

The methods and compositions described herein provide may be used for detecting one or more targets (e.g. one or more analytes) in a sample (e.g. a tissue sample) and/or identifying the one or more targets (e.g. one or more analytes). In some cases, the one or more targets (e.g. one or more analytes) may be detected/identified in situ. To identify one or more targets (e.g. one or more analytes), the signals detected within one or more detection cycles should involve a low error rate. Additionally, to identify one or more targets (e.g. analytes) in situ, there are advantages to detecting the information needed to identify one or more targets in a faster time (e.g. by using fewer detection cycles). The methods and compositions described herein may provide certain advantages compared to other technologies for detecting one or more targets (e.g. analytes) within a sample. For example, compared to bulk measurements, the methods and compositions described herein may provide information regarding both the identity of the one or more targets (e.g. analytes) and their spatial context. Additionally, compared to other in situ detection technologies, the methods and compositions described herein may enable highly multiplexed readouts with low error rates in a high throughput manner. For example, the methods and compositions described herein may provide information relevant to identifying one or more targets (e.g. analytes) during a given detection cycle. The detection cycle may comprise more information useful for identifying one or more targets (e.g. analytes) within a cycle than other in situ methods. The information in a detection cycle may have lower error rates relative to other detection methods of other in situ technologies.

The methods described herein may comprise identifying one or more analytes. The identifying of the one or more analytes may comprise using the one or more subsequences to compare to a codebook or database. The codebook or database may comprise a correlation between one or more analytes and one or more subsequences and/or sequence combinations. In some cases, an analyte of the one or more analytes may be represented by a combination of subsequences that are unique to that analyte and can be used to identify the analyte (e.g. a barcode or sequence comprising a combination of subsequences). For example, a protein analyte may be identified by detecting and/or identifying a combination of subsequences. The protein analyte may be identified by contacting the sample with one or more binding moieties. The one or more binding moieties may comprise antibodies conjugated to oligonucleotides. The sample may be further contacted with one or more binders (e.g. one or more padlock probes). The one or more binders may comprise one or more subsequences. The one or more binders may be used for binding to/coupling to at least a portion of a sequence of a binding moiety, and/or amplifying at least a portion of a sequence of a binding moiety. The one or more binders may comprise one or more subsequences that can be used to correlate the identify of one or more analytes recognized by one or more binding moieties. The one or more binders may be ligated upon binding to oligonucleotides of the one or more binding moieties. The one or more binders may be amplified by performing an amplification reaction (e.g. a rolling circle amplification reaction). The amplification reaction may generate a derivative of the one or more binders (e.g. a reverse compliment or complement of the binders). The subsequences (or complement or reverse complement thereof) may be detected and/or identified by detecting one or more signals and/or counts associated with one or more detection probes as described in the methods disclosed herein. The subsequences identified and/or detected across one or more rounds of detection may be compared to combinations of subsequences within a database or codebook to identify the analyte.

The methods described herein may include certain advantages compared to other methods. In some cases, the methods described herein may enable identification of a number of analytes using fewer detection cycles compared to other methods. For example, the methods described herein may use at least a 2-state detection scheme, at least a 3-state detection scheme, at least a 4-state detection scheme, at least a 5-state detection scheme, at least a 6-state detection scheme, at least a 7-state detection scheme, least a 8-state detection scheme, at least a 9-state detection scheme, at least a 10-state detection scheme, at least a 11-state detection scheme, at least a 12-state detection scheme, at least a 13-state detection scheme, at least a 14-state detection scheme, at least a 15-state detection scheme, at least a 16-state detection scheme, at least a 17-state detection scheme, at least a 18-state detection scheme, at least a 19-state detection scheme, at least a 20-state detection scheme, or more. The methods described herein may use at most a 2-state detection scheme, at most a 3-state detection scheme, at most a 4-state detection scheme, at most a 5-state detection scheme, at most a 6-state detection scheme, at most a 7-state detection scheme, most a 8-state detection scheme, at most a 9-state detection scheme, at most a 10-state detection scheme, at most a 11-state detection scheme, at most a 12-state detection scheme, at most a 13-state detection scheme, at most a 14-state detection scheme, at most a 15-state detection scheme, at most a 16-state detection scheme, at most a 17-state detection scheme, at most a 18-state detection scheme, at most a 19-state detection scheme, at most a 20-state detection scheme, or more. The methods described herein may use about a 2-state to about a 20-state detection scheme, about a 3-state to about a 19-state detection scheme, about a 4-state to about a 18-state detection scheme, about a 5-state to about a 17-state detection scheme, about a 6-state to about a 16-state detection scheme, about a 7-state to about a 15-state detection scheme, about a 8-state to about a 14-state detection scheme, about a 9-state to about a 13-state detection scheme, or about a 10-state to about a 12-state detection scheme.

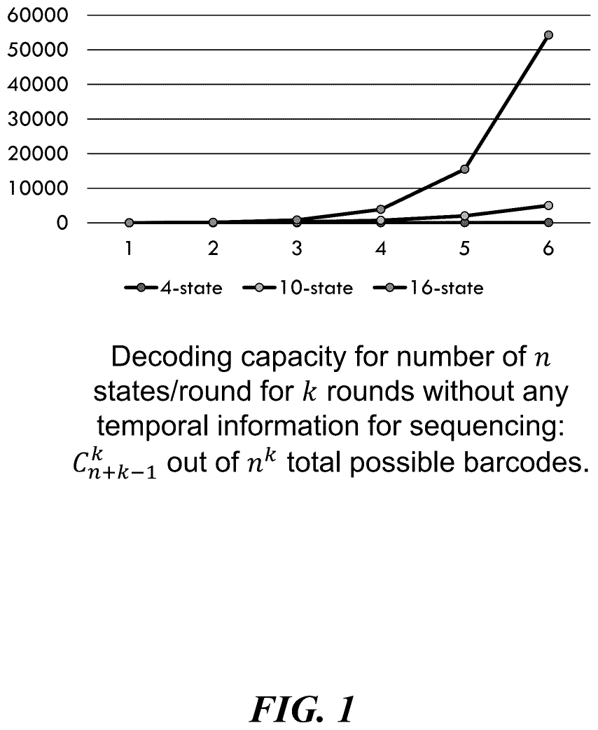

The number of state pertains to the number of types of subsequences that may be detected in a given codebook. For example, a 4-state experiment may comprise the following nucleotides: A, C, T, and G. The decoding capacity may scale according to the traces shown in FIG. 1 for a 4-state, 10-state, and 16-state setup for a given set of analytes across a given set of cycles. Performing fewer cycles may take less time. The methods described herein can enable an efficient and/or fast detection method for identifying many targets in part because fewer cycles are needed. In some cases, a 2-state, 3-state, 4-state, 5-state, 6-state, 7-state, 8-state, 9-state, 10-state, 11-state, 12-state, 13-state, 14-state, 15-state, 16-state, 17-state, 18-state, 19-state, or 20-state code may be used to detect at least about 5 analytes, at least about 10 analytes, at least about 15 analytes, at least about 20 analytes, at least about 25 analytes, at least about 30 analytes, at least about 35 analytes, at least about 40 analytes, at least about 45 analytes, at least about 50 analytes, at least about 55 analytes, at least about 60 analytes, at least about 65 analytes, at least about 70 analytes, at least about 75 analytes, at least about 80 analytes, at least about 85 analytes, at least about 90 analytes, at least about 95 analytes, at least about 100 analytes, at least about 150 analytes, at least about 200 analytes, at least about 250 analytes, at least about 300 analytes, at least about 350 analytes, at least about 400 analytes, at least about 450 analytes, at least about 500 analytes, at least about 550 analytes, at least about 600 analytes, at least about 650 analytes, at least about 700 analytes, at least about 750 analytes, at least about 800 analytes, at least about 850 analytes, at least about 900 analytes, at least about 950 analytes, at least about 1000 analytes, at least about 2000 analytes, at least about 3000 analytes, at least about 4000 analytes, at least about 5000 analytes, at least about 6000 analytes, at least about 7000 analytes, at least about 8000 analytes, at least about 9000 analytes, at least about 10000 analytes, at least about 11000 analytes, at least about 12000 analytes, at least about 13000 analytes, at least about 14000 analytes, at least about 15000 analytes, at least about 16000 analytes, at least about 17000 analytes, at least about 18000 analytes, at least about 19000 analytes, at least about 20000 analytes, at most about 5 analytes, at most about 10 analytes, at most about 15 analytes, at most about 20 analytes, at most about 25 analytes, at most about 30 analytes, at most about 35 analytes, at most about 40 analytes, at most about 45 analytes, at most about 50 analytes, at most about 55 analytes, at most about 60 analytes, at most about 65 analytes, at most about 70 analytes, at most about 75 analytes, at most about 80 analytes, at most about 85 analytes, at most about 90 analytes, at most about 95 analytes, at most about 100 analytes, at most about 150 analytes, at most about 200 analytes, at most about 250 analytes, at most about 300 analytes, at most about 350 analytes, at most about 400 analytes, at most about 450 analytes, at most about 500 analytes, at most about 550 analytes, at most about 600 analytes, at most about 650 analytes, at most about 700 analytes, at most about 750 analytes, at most about 800 analytes, at most about 850 analytes, at most about 900 analytes, at most about 950 analytes, at most about 1000 analytes, at most about 2000 analytes, at most about 3000 analytes, at most about 4000 analytes, at most about 5000 analytes, at most about 6000 analytes, at most about 7000 analytes, at most about 8000 analytes, at most about 9000 analytes, at most about 10000 analytes, at most about 11000 analytes, at most about 12000 analytes, at most about 13000 analytes, at most about 14000 analytes, at most about 15000 analytes, at most about 16000 analytes, at most about 17000 analytes, at most about 18000 analytes, at most about 19000 analytes, or at most about 20000 analytes, using at least about 1 detection cycle, at least about 2 detection cycles, at least about 3, at least about 4 detection cycles, at least about 5 detection cycles, 6 detection cycles, at least about 7 detection cycles, at least about 8 detection cycles, at least about 9 detection cycles, at least about 10 detection cycles, at least about 11 detection cycles, at least about 12 detection cycles, at least about 13 detection cycles, at least about 14 detection cycles, at least about 15 detection cycles, at least about 16 detection cycles, at least about 17 detection cycles, at least about 18 detection cycles, at least about 19 detection cycles, at least about 20 detection cycles, at least about 21 detection cycles, at least about 22 detection cycles, at least about 23 detection cycles, at least about 24 detection cycles, at least about 25 detection cycles, at least about 26 detection cycles, at least about 27 detection cycles, at least about 28 detection cycles, at least about 29 detection cycles, at least about 30 detection cycles, at least about 31 detection cycles, at least about 32 detection cycles, at least about 33 detection cycles, at least about 34 detection cycles, at least about 35 detection cycles, at least about 36 detection cycles, at least about 37 detection cycles, at least about 38 detection cycles, at least about 39 detection cycles, at least about 40 detection cycles, at least about 41 detection cycles, at least about 42 detection cycles, at least about 43 detection cycles, at least about 44 detection cycles, at least about 45 detection cycles, at least about 47 detection cycles, at least about 48 detection cycles, at least about 49 detection cycles, at least about 50 detection cycles, or more. In some cases, detection of between 1 and 20,000 or more analytes is accomplished using at most about 1 detection cycle, at most about 2 detection cycles, at most about 3, at most about 4 detection cycles, at most about 5 detection cycles, 6 detection cycles, at most about 7 detection cycles, at most about 8 detection cycles, at most about 9 detection cycles, at most about 10 detection cycles, at most about 11 detection cycles, at most about 12 detection cycles, at most about 13 detection cycles, at most about 14 detection cycles, at most about 15 detection cycles, at most about 16 detection cycles, at most about 17 detection cycles, at most about 18 detection cycles, at most about 19 detection cycles, at most about 20 detection cycles, at most about 21 detection cycles, at most about 22 detection cycles, at most about 23 detection cycles, at most about 24 detection cycles, at most about 25 detection cycles, at most about 26 detection cycles, at most about 27 detection cycles, at most about 28 detection cycles, at most about 29 detection cycles, at most about 30 detection cycles, at most about 31 detection cycles, at most about 32 detection cycles, at most about 33 detection cycles, at most about 34 detection cycles, at most about 35 detection cycles, at most about 36 detection cycles, at most about 37 detection cycles, at most about 38 detection cycles, at most about 39 detection cycles, at most about 40 detection cycles, at most about 41 detection cycles, at most about 42 detection cycles, at most about 43 detection cycles, at most about 44 detection cycles, at most about 45 46 detection cycles, at most about 47 detection cycles, at most about 48 detection cycles, at most about 49 detection cycles, or at most about 50 detection cycles.

An aspect of the disclosure provides a method for identifying subsequences in a sample. The method may comprise contacting the sample with a binding moiety. The binding moiety may comprise a probe. The probe may couple to the analyte. The binding moiety may comprise a sequence. The binding moiety may comprise a pre-determined sequence. The sequence may comprise one or more pluralities of subsequences. The one or more pluralities of subsequences may correlate to the analyte. In some cases, the method may comprise bringing the analyte in contact with one or more detection probes. For example, the method may comprise bringing the analyte in contact with at least a first detection probe, a second detection probe, or a combination thereof. In some cases, the first detection probe may couple to a first subsequence of the sequence or derivative thereof. In some cases, the first detection probe may comprise a first detection moiety. In some cases, the second detection probe may couple to a second subsequence of the sequence or derivative thereof. The second detection probe may comprise a second detection moiety. In some cases, the first subsequence and/or the second subsequence may be adjacent to each other. The method may comprise detecting the one or more detection moieties associated with the one or more detection probes (e.g., the first detection moiety, the second detection moiety, or a combination thereof) to identify at least the first subsequence, the second subsequence, or a combination thereof. The method may comprise using at least the first subsequence, the second subsequence, or combination thereof identified in the detecting to identify the analyte.

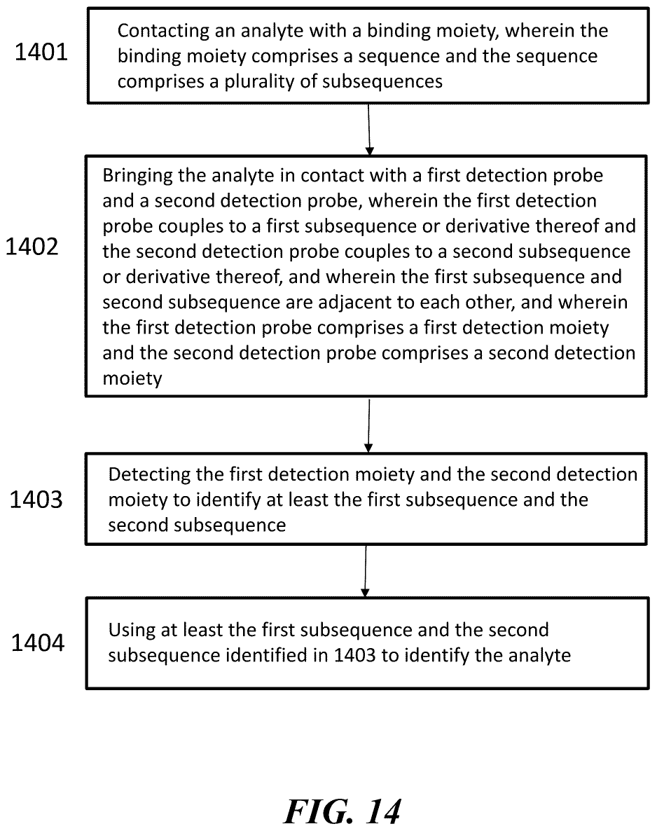

FIG. 14 schematically illustrates an example for identifying subsequences in a sample. In this example, an analyte is contacted with a binding moiety (1401). The binding moiety may comprise a probe that couples to the analyte, a sequence, or a combination thereof. The sequence may comprise one or more pluralities of subsequences. The analyte may be contacted with a first detection probe, a second detection probe, or a combination thereof (1402). The first detection probe may couple to a first subsequence of the sequence. The first detection probe may couple to a first subsequence of a derivative (e.g. a complement or a reverse complement) of the sequence. The first detection probe may comprise a detection moiety (e.g. a fluorescent dye). The second detection probe may couple to a second subsequence of the sequence. The second detection probe may couple to a second subsequence of a derivative (e.g. a complement or a reverse complement) of the sequence. The second detection probe may comprise a detection moiety (e.g. a fluorescent dye). The first subsequence and/or the second subsequence may be adjacent to each other (e.g. may be contiguous nucleotides within a nucleic acid sequence). The first detection moiety may be detected to identify the first subsequence (1403). The second detection moiety may be detected to identify the second subsequence (1403). The analyte may be identified using the identified first subsequence, the identified second subsequence, or a combination thereof (1404). FIG. 14 shows a process for identifying subsequences in a sample.

Another aspect of the disclosure provides a method for identifying subsequences in a sample. The method may comprise contacting the sample with a binding moiety. The binding moiety may comprise a probe. The probe may couple to the analyte. The binding moiety may comprise a sequence. The sequence may comprise one or more pluralities of subsequences. In some cases, the method may comprise bringing the analyte in contact with a first detection probe, a second detection probe, or a combination thereof. In some cases, the first detection probe may couple to a first subsequence of the sequence or derivative thereof. In some cases, the first detection probe may comprise a first detection moiety. In some cases, the second detection probe may couple to a second subsequence of the sequence or derivative thereof. The second detection probe may comprise a second detection moiety. In some cases, the first subsequence may be covalently linked to the second subsequence. The method may comprise detecting the first detection moiety, the second detection moiety, or a combination thereof to identify at least the first subsequence, the second subsequence, or a combination thereof. The method may comprise using at least the first subsequence, the second subsequence, or combination thereof identified in the detecting to identify the analyte.

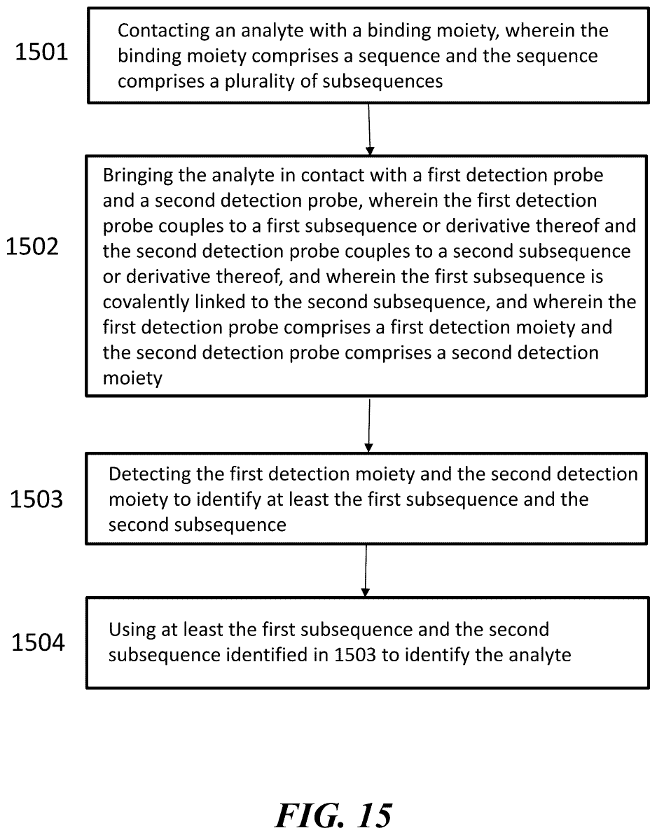

FIG. 15 schematically illustrates an example for identifying subsequences in a sample. In this example, an analyte is contacted with a binding moiety (1501). The binding moiety may comprise a probe that couples to the analyte, a sequence, or a combination thereof. The sequence may comprise one or more pluralities of subsequences. The analyte may be contacted with a first detection probe, a second detection probe, or a combination thereof (1502). The first detection probe may couple to a first subsequence of the sequence. The first detection probe may couple to a first subsequence of a derivative (e.g. a complement or a reverse complement) of the sequence. The first detection probe may comprise a detection moiety (e.g. a fluorescent dye). The second detection probe may couple to a second subsequence of the sequence. The second detection probe may couple to a second subsequence of a derivative (e.g. complement and/or reverse complement) of the sequence. The second detection probe may comprise a detection moiety (e.g. a fluorescent dye). The first subsequence may be covalently linked to the second subsequence. The first detection moiety may be detected to identify the first subsequence (1503). The second detection moiety may be detected to identify the second subsequence (1503). The analyte may be identified using the identified first subsequence, the identified second subsequence, or a combination thereof (1504). FIG. 15 shows a process for identifying subsequences in a sample.

Another aspect of the disclosure provides a method for identifying subsequences in a sample. The method may comprise contacting the sample with a binding moiety. The binding moiety may comprise a probe. The probe may couple to the analyte. The binding moiety may comprise a sequence. The method may comprise contacting the sample a binder. The binder may couple to or bind to the binding moiety. For example, the binder may couple to or bind to the sequence of the binding moiety. The binder may comprise one or more subsequences. The one or more subsequences may correlate with (e.g. code for) the analyte. In some cases, the method may comprise bringing the analyte in contact with one or more detection probes. For example, the method may comprise bringing the analyte in contact with at least a first detection probe, a second detection probe, or a combination thereof. In some cases, the first detection probe may couple to a first subsequence of the binder or derivative thereof (e.g. complement or reverse complement thereof). In some cases, the first detection probe may comprise a first detection moiety. In some cases, the second detection probe may couple to a second subsequence of the binder or derivative thereof (e.g. complement or reverse complement thereof). The second detection probe may comprise a second detection moiety. In some cases, the first subsequence and/or the second subsequence may be adjacent to each other. The method may comprise detecting the one or more detection moieties associated with the one or more detection probes (e.g., the first detection moiety, the second detection moiety, or a combination thereof) to identify at least the first subsequence, the second subsequence, or a combination thereof. The method may comprise using at least the first subsequence, the second subsequence, or combination thereof identified in the detecting to identify the analyte.

Another aspect of the disclosure provides a method for identifying subsequences in a sample. The method may comprise contacting the sample with a binding moiety. The binding moiety may comprise a probe. The probe may couple to the analyte. The binding moiety may comprise a sequence. The method may comprise contacting the sample a binder. The binder may couple to or bind to the binding moiety. For example, the binder may couple to or bind to the sequence of the binding moiety. The binder may comprise one or more subsequences. The one or more subsequences may correlate with (e.g. code for) the analyte. In some cases, the method may comprise bringing the analyte in contact with one or more detection probes. For example, the method may comprise bringing the analyte in contact with at least a first detection probe, a second detection probe, or a combination thereof. In some cases, the first detection probe may couple to a first subsequence of the binder or derivative thereof (e.g. complement or reverse complement thereof). In some cases, the first detection probe may comprise a first detection moiety. In some cases, the second detection probe may couple to a second subsequence of the binder or derivative thereof (e.g. complement or reverse complement thereof). The second detection probe may comprise a second detection moiety. In some cases, the first subsequence may be covalently linked to the second subsequence. The method may comprise detecting the one or more detection moieties associated with the one or more detection probes (e.g., the first detection moiety, the second detection moiety, or a combination thereof) to identify at least the first subsequence, the second subsequence, or a combination thereof. The method may comprise using at least the first subsequence, the second subsequence, or combination thereof identified in the detecting to identify the analyte.

The methods described herein may have certain advantages that result in a high accuracy of detection. For example, a first detection probe (e.g. the first detection probe) and a second detection probe (e.g. the second detection probe) may bind to or couple to the binding moiety. The combination of these binding events may enable a ligation event. The ligation event may generate a strong interaction between the first and second detection probes and the binding moiety. The strong interaction between the first and second detection probes and the binding moiety may enable more accurate detection of the detection probes. The binding of the first and second detection probes may result in more specific detection of the subsequences (e.g. a specificity of at least about 60%, at least about 65%, at least about 70%, at least about 75%, at least about 80%, at least about 85%, at least about 90%, at least about 95%, at least about 96%, at least about 97%, at least about 98%, at least about 99%, or more). The more specific detection may be a result of not detecting detection probes that are not ligated when bound to the binding moiety. In another example, a first detection probe (e.g. the first detection probe) and a second detection probe (e.g. the second detection probe) may bind to or couple to a binder or an amplification product of a binder (e.g. one or more amplicons). The combination of these binding events may enable a ligation event. The ligation event may generate a strong interaction between the first and second detection probes and the binder or the amplification product of the binder (e.g. one or more amplicons). The strong interaction between the first and second detection probes and the binder or the amplification product of the binder may enable more accurate detection of the detection probes. The binding of the first and second detection probes may result in more specific detection of the subsequences (e.g. a specificity of at least about 60%, at least about 65%, at least about 70%, at least about 75%, at least about 80%, at least about 85%, at least about 90%, at least about 95%, at least about 96%, at least about 97%, at least about 98%, at least about 99%, or more). The more specific detection may be a result of not detecting detection probes that are not ligated when bound to the binder or the amplification product of the binder. In some cases, one or more detection probes may bind to or couple to one or more amplification products (e.g. one or more amplicons) within a sample comprising one or more analytes. The one or more amplification products may be generated by: a) ligating a first end of a padlock probe to a second end of a padlock probe to form a circular nucleic acid; b) contracting the sample with a primer that couples to the circular nucleic acid; and c) amplifying the circular nucleic acid to generate one or more amplification products (e.g. one or more amplicons). The ligating of the first of a padlock probe to a second end of a padlock probe may be enabled by binding the padlock probe to a primer. The primer may comprise one or more binding sites that bind to or couple to the padlock probe.

Another aspect of the disclosure provides a method of analyzing analytes in a sample. The method may comprise providing a sample. The sample may comprise one or more analytes. The one or more analytes may comprise one or more nucleic acids. The one or more analytes may comprise one or more polypeptides. The method may comprise contacting the one or more analytes with one or more binding moieties. The method may comprise contacting the one or more analytes with one or more detection probes. The binding moiety may comprise one or more sequences. The method may comprise detecting at least a portion of the sequence or derivative there (e.g. complement thereof or reverse complement thereof). The detection may have greater than 50% (e.g. at least 70%) accuracy. The at least a portion of the sequence or derivative thereof may be detected when two or more detection probes are adjacent to one another.

The accuracy of detection may be measured by a variety of metrics, including but not limited to specificity, sensitivity, detecting the correct barcode or complement thereof, detecting the correct barcode or complement or reverse complements thereof in comparison to detecting incorrect barcodes or complements or reverse complements thereof, or a combination thereof. In some cases, the accuracy of detection may be at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%. In some cases, the accuracy of detection may be at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 96%, at most about 97%, at most about 98%, at most about 99%, or at most about 100%.

In some examples, one or more subsequences or complements or reverse complements thereof (e.g. the first subsequence and the second subsequence) may be adjacent to each other. For example, the first subsequence or complement or reverse complement thereof may be directly next to the second subsequence or complement or reverse complement thereof without any unit or subsequence or complement or reverse complement thereof separating the first subsequence or complement or reverse complement thereof from the second subsequence or complement or reverse complement thereof. In some cases, the first subsequence or complement or reverse complement thereof and/or the second subsequence or complement or reverse complement thereof may comprise nucleotides of a nucleic acid molecule. The nucleotides of the first subsequence or complement or reverse complement thereof may be directly sequential to the nucleotides of the second subsequence or complement or reverse complement thereof. In some cases, the first subsequence or complement or reverse complement thereof may be linked the second subsequence or complement or reverse complement thereof (e.g. covalently linked). The first subsequence or complement or reverse complement thereof may be linked to the second subsequence using one or more linkages. The one or more linkages may comprise one or more phosphodiester linkages, one or more amide linkages, one or more peg linkages, one or more ester linkages, one or more ether linkages, one or more methylene linkages, or a combination thereof. For example, the first subsequence and/or the second subsequence may comprise nucleic acid sequences and the nucleic acid sequence of the first subsequence may be linked to the nucleic acid sequence of the second subsequence via one or more phosphodiester linkages. In some cases, the first subsequence and the second subsequence may be separated by one or more nucleotides. For example, the first subsequence and the second subsequence may be separated by at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100, or more nucleotides. In some cases, the first subsequence and the second subsequence may be separated by at most about 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, at most about 20 nucleotides, at most about 21 nucleotides, at most about 22 nucleotides, at most about 23 nucleotides, at most about 24 nucleotides, at most about 25 nucleotides, at most about 30 nucleotides, at most about 35 nucleotides, at most about 40 nucleotides, at most about 45 nucleotides, at most about 50 nucleotides, at most about 55 nucleotides, at most about 60 nucleotides, at most about 65 nucleotides, at most about 70 nucleotides, at most about 75 nucleotides, at most about 80 nucleotides, at most about 85 nucleotides, at most about 90 nucleotides, at most about 95 nucleotides, at most about 100, or fewer nucleotides. The first subsequence may comprise one or more nucleotides, one or more amino acids, one or more chemical groups, or a combination thereof. For example, the first subsequence and/or the second subsequence may be part of a nucleic acid sequence. The one or more nucleotides of the first subsequence may comprise one or more naturally occurring nucleotides, one or more non-naturally occurring nucleotides, or a combination thereof. The one or more nucleotides of the first subsequence may comprise one or more adenines (A), one or more guanines (G), one or more thymines (T), one or more cytosines (C), one or more uracils (U), one or more xanthines, one or more hypoxanthines, one or more 8-azapurines, one or more purines substituted at the 8 position with methyl or bromine, 9-oxo-N6-methyladenine, one or more 2-aminoadenines, one or more 7-deazaxanthines, one or more 7-deazaguanines, one or more 7-deaza-adenines, one or more N4-ethanocytosines, 2,6-diaminopurines, one or more N6-ethano-2,6-diaminopurines, one or more 5-methylcytosines, one or more 5-(C3-C6)-alkynylcytosines, one or more 5-alkynyluracils, one or more 5-fluorouracils, one or more 5-bromouracils, one or more thiouracils, one or more pseudoisocytosines, one or more 2-hydroxy-5-methyl-4-triazolopyridines, one or more isocytosines, one or more isoguanines, one or more inosines, one or more 7,8-dimethylalloxazines, one or more 6-dihydrothymines, one or more 5,6-dihydrouracils, one or more 4-methyl-indoles, ethenoadenines, or a combination thereof.

The second subsequence may comprise one or more nucleotides, one or more amino acids, one or more chemical groups, or a combination thereof. For example, the first subsequence and/or the second subsequence may be part of a nucleic acid sequence. The one or more nucleotides of the second subsequence may comprise one or more naturally occurring nucleotides, one or more non-naturally occurring nucleotides, or a combination thereof. The one or more nucleotides of the second subsequence may comprise one or more adenines, one or more guanines, one or more thymines, one or more cytosines, one or more uracils, one or more xanthines, one or more hypoxanthines, one or more 8-azapurines, one or more purines substituted at the 8 position with methyl or bromine, 9-oxo-N6-methyladenine, one or more 2-aminoadenines, one or more 7-deazaxanthines, one or more 7-deazaguanines, one or more 7-deaza-adenines, one or more N4-ethanocytosines, 2,6-diaminopurines, one or more N6-ethano-2,6-diaminopurines, one or more 5-methylcytosines, one or more 5-(C3-C6)-alkynylcytosines, one or more 5-alkynyluracils, one or more 5-fluorouracils, one or more 5-bromouracils, one or more thiouracils, one or more pseudoisocytosines, one or more 2-hydroxy-5-methyl-4-triazolopyridines, one or more isocytosines, one or more isoguanines, one or more inosines, one or more 7,8-dimethylalloxazines, one or more 6-dihydrothymines, one or more 5,6-dihydrouracils, one or more 4-methyl-indoles, ethenoadenines, or a combination thereof.

In some aspects, the methods described herein may comprise ligating one detection probe to another detection probe (for example ligating the first detection probe to the second detection probe). The ligating may comprise using one or more ligases. For example, a sample comprising the analyte and a first detection probe and/or a second detection probe may be contacted with a ligase and/or buffers and/or reagents. In some cases, the one or more ligases may comprise a mammalian ligase. In some cases, the one or more ligases may comprise a bacterial ligase. The one or more ligases may comprise a deoxyribonucleic acid (DNA) ligase I, DNA ligase II, DNA ligase III, DNA ligase IV, or a combination thereof. In some cases, the one or more ligases may comprise an RNA ligase. In some cases, the ligase may ligate a 3′ nucleotide of one nucleic acid detection probe to a 5′ nucleotide of a different nucleic acid detection probe (e.g. not the same nucleic acid detection probe as the one nucleic acid detection probe). In some cases, the ligase may ligate a 3′ end of a nucleic acid detection probe to a 5′ end of the same nucleic acid detection probe. The one or more ligases may ligate two nucleotides that are part of a double-stranded nucleic acid. For example, the double-stranded nucleic acid may comprise a nick, and the location of the nick may be ligated by the one or more ligases. In some embodiments, the double-stranded nucleic acid may comprise a DNA/DNA duplex. In some embodiments, the double-stranded nucleic acid may comprise an RNA/DNA duplex. The ligase may comprise one or more of the following: T4 DNA ligase, SplintR ligase, T3 DNA ligase, T7 DNA ligase, E. coli DNA ligase, Taq ligase, RtcB ligase, or a combination thereof. In some cases, a ligase may be used to ligate a first end of a binder to a second end of a binder. In some cases, the binding moieties as described herein may comprise a nucleic acid (e.g. a padlock probe). A first end of binding moiety padlock probe may be ligated to a second end of the binding moiety padlock probe thereby generating a circular nucleic acid. A ligation reaction may be carried out using the one or more ligases. The ligation reaction may involve incubating a sample comprising an analyte comprising one or more detection probes. The sample may be incubated with one or more ligases for a period of time, for example at least 10 minutes, at least 20 minutes, at least 30 minutes, at least 1 hour, at least 2 hours, or more. The sample may be incubated with one or more ligases for at most 10 minutes, at most 20 minutes, at most 30 minutes, at most 1 hour, at most 2 hours, or more. The ligation reaction may comprise incubation with one or more components, including but not limited to one or more buffers, one or more salts, one or more detergents, one or more solvents, one or more chaotropic reagents, or a combination thereof. The one or more buffers of the ligation reaction conditions may comprise MES (4-Morpholineethanesulfonic acid), Bis-Tris (Bis(2-hydroxyethyl)amino-tris(hydroxymethyl)methane), ADA, ACES, PIPES, MOSO, Bis-Tris Propane, BES, MOPS, TES, HEPES, DIPSO, MOBS, TAPSO, Tris, HEPPSO, POPSO, TEA, EPPS, Tricine, Gly-Gly, Bicine, HEPBS, TAPS, AMPD, TABS, AMPSO, CHES, CAPSO, AMP, CAPS, CAPS, Phosphate buffered saline, or a combination thereof. The one or more salts of the ligation reaction conditions may comprise NaCl, CaCl₂, MgCl₂, or a combination thereof. The one or more detergents of the ligation reaction conditions may comprise SDS, Triton X-100, CHAPS, NP-40, Tween-20, Digitonin, or a combination thereof. The one or more solvents of the ligation reaction may comprise methanol, ethanol, ethyl acetate, DMSO, acetonitriles, water, or a combination thereof. The one or more chaotropic agents of the ligation reaction conditions may comprise DMSO, formamide, urea, thiourea, 2-propanol, guanidinium chloride, n-butanol, or a combination thereof. The ligation reaction may include an incubation at one or more temperatures. The one or more temperatures of the ligation reaction may be at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., at least about 71° C., at least about 72° C., at least about 73° C., at least about 74° C., at least about 75° C., at least about 76° C., at least about 77° C., at least about 78° C., at least about 79° C., at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., or higher. The one or more temperature may be at most about 4° C., at most about 5° C., at most about 6° C., at most about 7° C., at most about 8° C., at most about 9° C., at most about 10° C., at most about 11° C., at most about 12° C., at most about 13° C., at most about 14° C., at most about 15° C., at most about 16° C., at most about 17° C., at most about 18° C., at most about 19° C., at most about 20° C., at most about 21° C., at most about 22° C., at most about 23° C., at most about 24° C., at most about 25° C., at most about 26° C., at most about 27° C., at most about 28° C., at most about 29° C., at most about 30° C., at most about 31° C., at most about 32° C., at most about 33° C., at most about 34° C., at most about 35° C., at most about 36° C., at most about 37° C., at most about 38° C., at most about 39° C., at most about 40° C., at most about 41° C., at most about 42° C., at most about 43° C., at most about 44° C., at most about 45° C., at most about 46° C., at most about 47° C., at most about 48° C., at most about 49° C., at most about 50° C., at most about 51° C., at most about 52° C., at most about 53° C., at most about 54° C., at most about 55° C., at most about 56° C., at most about 57° C., at most about 58° C., at most about 59° C., at most about 60° C., at most about 61° C., at most about 62° C., at most about 63° C., at most about 64° C., at most about 65° C., at most about 66° C., at most about 67° C., at most about 68° C., at most about 69° C., at most about 70° C., at most about 71° C., at most about 72° C., at most about 73° C., at most about 74° C., at most about 75° C., at most about 76° C., at most about 77° C., at most about 78° C., at most about 79° C., at most about 80° C., at most about 81° C., at most about 82° C., at most about 83° C., at most about 84° C., at most about 85° C., at most about 86° C., at most about 87° C., at most about 88° C., at most about 89° C., at most about 90° C., at most about 91° C., at most about 92° C., at most about 93° C., at most about 94° C., at most about 95° C., or lower. The one or more temperatures may be about 4-95° C., about 5-94° C., about 6-93° C., about 7-92° C., about 8-91° C., about 9-90° C., about 10-89° C., about 11-88° C., about 12-87° C., about 13-86° C., about 14-85° C., about 15-84° C., about 16-83° C., about 17-82° C., about 18-81° C., about 19-80° C., about 20-79° C., about 21-78° C., about 22-77° C., about 23-76° C., about 24-75° C., about 25-74° C., about 26-73° C., about 27-72° C., about 28-71° C., about 29-70° C., about 30-69° C., about 31-68° C., about 32-67° C., about 33-66° C., about 34-65° C., about 35-64° C., about 36-63° C., about 37-62° C., about 38-61° C., about 39-60° C., about 40-59° C., about 41-58° C., about 42-57° C., about 43-56° C., about 44-55° C., about 45-54° C., about 46-53° C., about 47-52° C., about 48-51° C., or about 49-50° C.

In some cases, the ligating may comprise a chemical reaction between a chemical moiety on one detection probe and a chemical moiety on a second detection probe (e.g. the first detection probe and/or the second detection probe). The first detection probe may comprise a first chemical reactive moiety. The second detection probe may comprise a second chemical reactive moiety. The first chemical reactive moiety of the first detection probe may comprise a thioether, an acetal, an acyl halide, an alcohol, an aldehyde, an amide, an amidine, an amines, an azide, an azo compound, a borinic acid, a borinic ester, a boronic acid, a boronic ester, a carbamate ester, a carbonate, a carboxylate, a carboxylic acid, a cyanates, a disulfide, a dithiocarboxylic acid, an ester, an ether, a guanidine, a hemiacetal, a hemiketal, a heterocycle, a hydroperoxide, an imide, an imine, a ketal (or acetal), a ketone, a nitrate, a nitrile, a nitrite, a nitro compound, a nitroso compound, an organic acid anhydride, an orthocarbonate ester, an orthoester, an oxime, a peroxide, a pyridine derivative, a sulfide, a sulfinic acid, a sulfonate ester, a sulfone, a sulfonic acid, a sulfoxide, a thial, a thiocarboxylic acid, a thiocyanate, a thioester, a thioketone, a thiol, a tetrazine, an alkyne, a trans-cyclooctene, a maleimide, a n-hydroxysuccinimide ester, a hydroxyl, a cyclopropenone, a norbornene, or a combination thereof. The second chemical reactive moiety may comprise a thioether, an acetal, an acyl halide, an alcohol, an aldehyde, an amide, an amidine, an amines, an azide, an azo compound, a borinic acid, a borinic ester, a boronic acid, a boronic ester, a carbamate ester, a carbonate, a carboxylate, a carboxylic acid, a cyanates, a disulfide, a dithiocarboxylic acid, an ester, an ether, a guanidine, a hemiacetal, a hemiketal, a heterocycle, a hydroperoxide, an imide, an imine, a ketal (or acetal), a ketone, a nitrate, a nitrile, a nitrite, a nitro compound, a nitroso compound, an organic acid anhydride, an orthocarbonate ester, an orthoester, an oxime, a peroxide, a pyridine derivative, a sulfide, a sulfinic acid, a sulfonate ester, a sulfone, a sulfonic acid, a sulfoxide, a thial, a thiocarboxylic acid, a thiocyanate, a thioester, a thioketone, a thiol, a tetrazine, an alkyne, a trans-cyclooctene, a maleimide, a n-hydroxysuccinimide ester, a hydroxyl, a cyclopropenone, a norbornene, or a combination thereof. The ligating may comprise reacting the first chemical reactive chemical moiety to the second reactive chemical moiety to form a covalent bond.

In some aspects, the sample comprising the analyte may comprise a biological specimen extracted from a subject. The subject may be a mammal, a bacteria, a fungus, or a combination there. The subject may be a human subject. The subject may be a mouse. In some cases, the sample may comprise cells grown ex vivo, e.g., using cell culture methods. In some embodiments, the sample may comprise a cell, a tissue, a bodily fluid, or a combination thereof.

The sample comprising the analyte may be a variety of formats and/or may comprise a variety or features and/or characteristics. The sample may comprise one or more cells. In some embodiments, the sample may comprise one or more cells, one or more tissue samples, one or more bodily fluids, or a combination thereof. The cells of the sample may be cultured cells. The cultured cells may be cultured in vivo, ex vivo or in vitro. The sample may comprise a tissue sample. The tissue sample may be fresh, fresh-frozen, fixed, fixed-frozen, formalin-fixed, paraffin embedded, or a combination thereof. In some cases, the tissue sample may be fixed using a cross-linking reagent. In some cases, the tissue sample may be fixed using a preservative. In some cases, the cross-linking reagent may comprise formaldehyde, formalin, glutaraldehyde, or a combination thereof.

The sample may comprise a tissue sample that has been sliced from a tissue block. The tissue sample may be immobilized onto a substrate. The substrate may be a well-plate, a slide, a coverslip, a well, a surface, a flow cell, or a combination thereof. The slide may be a microscope slide. The tissue sample may have a variety of thicknesses. The tissue sample may be at least at least about 1 μm thick, at least about 2 μm thick, at least about 3 μm thick, at least about 4 μm thick, at least about 5 μm thick, at least about 6 μm thick, at least about 7 μm thick, at least about 8 μm thick, at least about 9 μm thick, at least about 10 μm thick, at least about 11 μm thick, at least about 12 μm thick, at least about 13 μm thick, at least about 14 μm thick, at least about 15 μm thick, at least about 16 μm thick, at least about 17 μm thick, at least about 18 μm thick, at least about 19 μm thick, at least about 20 μm thick, at least about 21 μm thick, at least about 22 μm thick, at least about 23 μm thick, at least about 24 μm thick, at least about 25 μm thick, at least about 26 μm thick, at least about 27 μm thick, at least about 28 μm thick, at least about 29 μm thick, at least about 30 μm thick, at least about 31 μm thick, at least about 32 μm thick, at least about 33 μm thick, at least about 34 μm thick, at least about 35 μm thick, at least about 36 μm thick, at least about 37 μm thick, at least about 38 μm thick, at least about 39 μm thick, at least about 40 μm thick, at least about 41 μm thick, at least about 42 μm thick, at least about 43 μm thick, at least about 44 μm thick, at least about 45 μm thick, at least about 46 μm thick, at least about 47 μm thick, at least about 48 μm thick, at least about 49 μm thick, at least about 50 μm thick, at least about 51 μm thick, at least about 52 μm thick, at least about 53 μm thick, at least about 54 μm thick, at least about 55 μm thick, at least about 56 μm thick, at least about 57 μm thick, at least about 58 μm thick, at least about 59 μm thick, at least about 60 μm thick, at least about 61 μm thick, at least about 62 μm thick, at least about 63 μm thick, at least about 64 μm thick, at least about 65 μm thick, at least about 66 μm thick, at least about 67 μm thick, at least about 68 μm thick, at least about 69 μm thick, at least about 70 μm thick, at least about 71 μm thick, at least about 72 μm thick, at least about 73 μm thick, at least about 74 μm thick, at least about 75 μm thick, at least about 76 μm thick, at least about 77 μm thick, at least about 78 μm thick, at least about 79 μm thick, at least about 80 μm thick, at least about 81 μm thick, at least about 82 μm thick, at least about 83 μm thick, at least about 84 μm thick, at least about 85 μm thick, at least about 86 μm thick, at least about 87 μm thick, at least about 88 μm thick, at least about 89 μm thick, at least about 90 μm thick, at least about 91 μm thick, at least about 92 μm thick, at least about 93 μm thick, at least about 94 μm thick, at least about 95 μm thick, at least about 96 μm thick, at least about 97 μm thick, at least about 98 μm thick, at least about 99 μm thick, at least about 100 μm thick, at least about 105 μm thick, at least about 110 μm thick, at least about 115 μm thick, at least about 120 μm thick, at least about 125 μm thick, at least about 130 μm thick, at least about 135 μm thick, at least about 140 μm thick, at least about 145 μm thick, at least about 150 μm thick, at least about 155 μm thick, at least about 160 μm thick, at least about 165 μm thick, at least about 170 μm thick, at least about 175 μm thick, at least about 180 μm thick, at least about 185 μm thick, at least about 190 μm thick, at least about 195 μm thick, at least about 200 μm thick, at least about 210 μm thick, at least about 220 μm thick, at least about 230 μm thick, at least about 240 μm thick, at least about 250 μm thick, at least about 260 μm thick, at least about 270 μm thick, at least about 280 μm thick, at least about 290 μm thick, at least about 300 μm thick, at least about 320 μm thick, at least about 340 μm thick, at least about 360 μm thick, at least about 380 μm thick, at least about 400 μm thick, at least about 420 μm thick, at least about 440 μm thick, at least about 460 μm thick, at least about 480 μm thick, at least about 500 μm thick, or more. The tissue sample may be at most at most about 1 μm thick, at most about 2 μm thick, at most about 3 μm thick, at most about 4 μm thick, at most about 5 μm thick, at most about 6 μm thick, at most about 7 μm thick, at most about 8 μm thick, at most about 9 μm thick, at most about 10 μm thick, at most about 11 μm thick, at most about 12 μm thick, at most about 13 μm thick, at most about 14 μm thick, at most about 15 μm thick, at most about 16 μm thick, at most about 17 μm thick, at most about 18 μm thick, at most about 19 μm thick, at most about 20 μm thick, at most about 21 μm thick, at most about 22 μm thick, at most about 23 μm thick, at most about 24 μm thick, at most about 25 μm thick, at most about 26 μm thick, at most about 27 μm thick, at most about 28 μm thick, at most about 29 μm thick, at most about 30 μm thick, at most about 31 μm thick, at most about 32 μm thick, at most about 33 μm thick, at most about 34 μm thick, at most about 35 μm thick, at most about 36 μm thick, at most about 37 μm thick, at most about 38 μm thick, at most about 39 μm thick, at most about 40 μm thick, at most about 41 μm thick, at most about 42 μm thick, at most about 43 μm thick, at most about 44 μm thick, at most about 45 μm thick, at most about 46 μm thick, at most about 47 μm thick, at most about 48 μm thick, at most about 49 μm thick, at most about 50 μm thick, at most about 51 μm thick, at most about 52 μm thick, at most about 53 μm thick, at most about 54 μm thick, at most about 55 μm thick, at most about 56 μm thick, at most about 57 μm thick, at most about 58 μm thick, at most about 59 μm thick, at most about 60 μm thick, at most about 61 μm thick, at most about 62 μm thick, at most about 63 μm thick, at most about 64 μm thick, at most about 65 μm thick, at most about 66 μm thick, at most about 67 μm thick, at most about 68 μm thick, at most about 69 μm thick, at most about 70 μm thick, at most about 71 μm thick, at most about 72 μm thick, at most about 73 μm thick, at most about 74 μm thick, at most about 75 μm thick, at most about 76 μm thick, at most about 77 μm thick, at most about 78 μm thick, at most about 79 μm thick, at most about 80 μm thick, at most about 81 μm thick, at most about 82 μm thick, at most about 83 μm thick, at most about 84 μm thick, at most about 85 μm thick, at most about 86 μm thick, at most about 87 μm thick, at most about 88 μm thick, at most about 89 μm thick, at most about 90 μm thick, at most about 91 μm thick, at most about 92 μm thick, at most about 93 μm thick, at most about 94 μm thick, at most about 95 μm thick, at most about 96 μm thick, at most about 97 μm thick, at most about 98 μm thick, at most about 99 μm thick, at most about 100 μm thick, at most about 105 μm thick, at most about 110 μm thick, at most about 115 μm thick, at most about 120 μm thick, at most about 125 μm thick, at most about 130 μm thick, at most about 135 μm thick, at most about 140 μm thick, at most about 145 μm thick, at most about 150 μm thick, at most about 155 μm thick, at most about 160 μm thick, at most about 165 μm thick, at most about 170 μm thick, at most about 175 μm thick, at most about 180 μm thick, at most about 185 μm thick, at most about 190 μm thick, at most about 195 μm thick, at most about 200 μm thick, at most about 210 μm thick, at most about 220 μm thick, at most about 230 μm thick, at most about 240 μm thick, at most about 250 μm thick, at most about 260 μm thick, at most about 270 μm thick, at most about 280 μm thick, at most about 290 μm thick, at most about 300 μm thick, at most about 320 μm thick, at most about 340 μm thick, at most about 360 μm thick, at most about 380 μm thick, at most about 400 μm thick, at most about 420 μm thick, at most about 440 μm thick, at most about 460 μm thick, at most about 480 μm thick, at most about 500 μm thick, or less. The tissue sample may be about 1 to about 500 μm thick, about 2 to about 480 μm thick, about 3 to about 460 μm thick, about 4 to about 440 μm thick, about 5 to about 420 μm thick, about 6 to about 400 μm thick, about 7 to about 380 μm thick, about 8 to about 360 μm thick, about 9 to about 340 μm thick, about 10 to about 320 μm thick, about 11 to about 300 μm thick, about 12 to about 290 μm thick, about 13 to about 280 μm thick, about 14 to about 270 μm thick, about 15 to about 260 μm thick, about 16 to about 250 μm thick, about 17 to about 240 μm thick, about 18 to about 230 μm thick, about 19 to about 220 μm thick, about 20 to about 210 μm thick, about 21 to about 200 μm thick, about 22 to about 195 μm thick, about 23 to about 190 μm thick, about 24 to about 185 μm thick, about 25 to about 180 μm thick, about 26 to about 175 μm thick, about 27 to about 170 μm thick, about 28 to about 165 μm thick, about 29 to about 160 μm thick, about 30 to about 155 μm thick, about 31 to about 150 μm thick, about 32 to about 145 μm thick, about 33 to about 140 μm thick, about 34 to about 135 μm thick, about 35 to about 130 μm thick, about 36 to about 125 μm thick, about 37 to about 120 μm thick, about 38 to about 115 μm thick, about 39 to about 110 μm thick, about 40 to about 105 μm thick, about 41 to about 100 μm thick, about 42 to about 99 μm thick, about 43 to about 98 μm thick, about 44 to about 97 μm thick, about 45 to about 96 μm thick, about 46 to about 95 μm thick, about 47 to about 94 μm thick, about 48 to about 93 μm thick, about 49 to about 92 μm thick, about 50 to about 91 μm thick, about 51 to about 90 μm thick, about 52 to about 89 μm thick, about 53 to about 88 μm thick, about 54 to about 87 μm thick, about 55 to about 86 μm thick, about 56 to about 85 μm thick, about 57 to about 84 μm thick, about 58 to about 83 μm thick, about 59 to about 82 μm thick, about 60 to about 81 μm thick, about 61 to about 80 μm thick, about 62 to about 79 μm thick, about 63 to about 78 μm thick, about 64 to about 77 μm thick, about 65 to about 76 μm thick, about 66 to about 75 μm thick, about 67 to about 74 μm thick, about 68 to about 73 μm thick, about 69 to about 72 μm thick, or about 70 to about 71 μm thick. In some cases, the tissue sample is about 5 to about 250 μm thick, about 10 to about 100 μm thick, or about 25 to about 150 μm thick.

The methods described herein may comprise detecting the analyte within the sample (e.g. a tissue sample). The methods described herein may comprise detecting the analyte on a tissue surface. In some cases, the methods described herein may comprise detecting an analyte outside of a tissue sample (e.g. a cytokine outside of a tissue sample). The sample may be embedded in a hydrogel. The hydrogel may be formed by polymerizing monomers in the presence of the sample. The hydrogel may comprise one or more polymers. The one or more polymers may comprise poly (vinyl alcohol) (PVA), poly (ethylene glycol) (PEG), poly (ethylene oxide) (PEO), poly (2-hydroxyethyl methacrylate) (PHEMA), poly (acrylic acid) (PAA), poly (acrylamide) (PAAm), or a combination thereof. Hydrogel may be formed during any step of the methods described herein. For example, the hydrogel may be formed prior to contacting the sample with one or more binding moieties. The hydrogel may be formed after contacting the sample with one or more binding moieties. In some cases, the hydrogel may be formed after contacting the sample with one or more probes but before contacting the sample with one or more other probes. The hydrogel may be formed before an amplification step. The hydrogel may be formed after an amplification step. The hydrogel may be formed before a ligation step between one or more detection probes. The hydrogel may be formed after a ligation step between one or more detection probes. The hydrogel may be formed before a gap-filling reaction. For example, the hydrogel may be formed and following the formation of the hydrogel, a gap-filling reaction may be performed. The gap-filling reaction may comprise generating a double-stranded nucleic acid between two previously double-stranded regions. For example, a padlock probe may couple or bind to a target within the sample. The padlock probe may couple or bind to the target such that two double-stranded regions are formed that flank (e.g. surround) a single-stranded region. The single-stranded region may be filled in (e.g. gap-filled) by extending the ends of the padlock probe to generate a double-stranded region that was previously a single-stranded region. The gap-filling reaction may be performed using a polymerase (e.g. a DNA polymerase). The product of the gap-filling reaction may be further ligated to form a circular nucleic acid, for example by adding a ligase. The hydrogel may be formed after a gap-filling reaction. The hydrogel may be formed before a detection step. The hydrogel may be formed after a detection step. The sample may be embedded into a hydrogel. The sample may be incubated for at least about 1 hour, at least about 12 hours, at least about a day, at least about two days, at least about three days, or longer before contacting the sample with one or more probes. The sample may be incubated for at most about 1 hour, at most about 12 hours, at most about a day, at most about two days, at most about three days, or less before contacting the sample with one or more probes.

The methods described herein may comprise identifying one or more analytes. A variety of different types of analytes may be identified, including but not limited to nucleic acids, polypeptides, lipids, small molecules, cells, or a combination thereof. In some cases, one or more analytes of the same type may be identified (e.g. one or more protein analytes). For example, two different proteins may be identified. In some cases, one or more analytes of different types of analytes may be analyzed (e.g. different analyte forms or compositions). For example, a protein and/or a nucleic acid may be identified.

In some aspects, the one or more analytes that are identified may comprise one or more polypeptides. The one or more polypeptides may comprise one or more proteins, one or more peptides, or a combination thereof. In some cases, the one or more polypeptides may comprise one or more proteins and the one or more proteins may comprise one or more enzymes, one or more antibodies, one or more transcription factors, one or more structural proteins, one or more defense proteins, one or more signaling proteins, one or more receptors, one or more soluble proteins, one or more transmembrane proteins or a combination thereof. In cases where the one or more proteins may comprise one or more signaling proteins, the one or more signaling proteins may comprise one or more cytokines, one or more chemokines, or a combination thereof. The one or more polypeptides may be implicated in a disease state or mechanism of interest. In cases where the one or more analytes that are identified comprise one or more polypeptides, the probe of the binding moiety may comprise an antibody, a nanobody, an antibody fragment, or combination thereof.

In some aspects, the one or more analytes that are identified may comprises one or more lipids. The one or more lipids may comprise one or more phospholipids, one or more sterols, one or more triglycerides or a combination thereof. In cases where the one or more analytes that are identified comprise one or more lipids, the probe of the biding moiety may comprise a moiety that detects, couples to, binds to, or otherwise recognizes the one or more lipids. For example, the probe may comprise an antibody, a nanobody, a lipid binding protein, or a combination thereof. In some cases, the lipid binding protein may comprise Lipid A.

In some aspects, the one or more analytes that are identified may comprises one or more small molecules. The one or more small molecules may comprise one or more signaling molecules, hormones, or a combination thereof. In cases where the one or more analytes that are identified comprise one or more small molecules, the probe of the biding moiety may comprise a moiety that detects, couples to, binds to, or otherwise recognizes the one or more small molecules. For example, the probe may comprise an antibody, a nanobody, or a combination thereof.

In some aspects, the one or more analytes that are identified may comprises one or more cells. The one or more cells may comprise a cell that expresses a marker unique to or indicative of the cell. For example, the one or more cells that are identified may comprise a cancer cell and the cancer cell may comprise an RNA and/or protein that is over or under expressed. The RNA and/or protein that is over or under expressed may be used to identify the cell as cancerous.

In some aspects, the analyte may comprise one or more nucleic acids. The one or more nucleic acids of the one or more analytes may comprise one or more deoxyribonucleic acids (DNA). The one or more nucleic acids of the one or more analytes may comprise one or more ribonucleic acids (RNA). In some embodiments, the one or more nucleic acids may comprise both DNA and RNA. In cases wherein the one or more nucleic acids may comprise one or more RNAs, the one or more RNAs may comprise a variety of types of RNAs, including but not limited to messenger RNA (mRNA), ribosomal RNA (rRNA), transfer RNA (tRNA), micro RNA (miRNA), or a combination thereof. The one or more nucleic acids may comprise one or more sequences native to the sample. For example, the one or more nucleic acids of the one or more analytes may comprise one or more endogenous RNAs. In some cases, the one or more nucleic acids of the one or more analytes may comprise one or more sequences exogenous to the sample. For example, the one or more sequences exogenous to the sample may have been delivered to the sample. The one or more nucleic acids of the one or more analytes may comprise one or more sequences associated with one or more therapeutic agents. For example, the one or more sequences associated with one or more therapeutic agents may comprise one or more sequences or portions thereof of one or more gene therapies, one or more CAR T cells, one or more genes or transcripts encoding an antibody, or a combination thereof.

In some embodiments, the one or more nucleic acids of the one or more analytes may comprise one or more modifications. In some cases, the one or more modifications of the one or more nucleic acids of the one or more analytes may be associated with a5′ end of a nucleic acid of the one or more nucleic acids, a 3′ end of one a nucleic acid of the one or more nucleic acids, one or more internal nucleotides of a nucleic acid of the one or more nucleic acids, or a combination thereof. The one or more modifications may comprise one or more phosphorylation groups, one or more methyl groups, one or more fluorescent modifications, one or more reactive chemical moieties, or a combination thereof. The one or more nucleic acids of the one or more analytes may comprise one or more nucleotides. The one of more nucleotides of the one or more nucleic acids of the one or more analytes may comprise one or more natural nucleotides, one or more non-natural nucleotides, or a combination thereof. The one of more nucleotides of the one or more nucleic acids of the one or more analytes may comprise one or more adenines, one or more guanines, one or more thymines, one or more cytosines, one or more uracils, one or more xanthines, one or more hypoxanthines, one or more 8-azapurines, one or more purines substituted at the 8 position with methyl or bromine, 9-oxo-N6-methyladenine, one or more 2-aminoadenines, one or more 7-deazaxanthines, one or more 7-deazaguanines, one or more 7-deaza-adenines, one or more N4-ethanocytosines, 2,6-diaminopurines, one or more N6-ethano-2,6-diaminopurines, one or more 5-methylcytosines, one or more 5-(C3-C6)-alkynylcytosines, one or more 5-alkynyluracils, one or more 5-fluorouracils, one or more 5-bromouracils, one or more thiouracils, one or more pseudoisocytosines, one or more 2-hydroxy-5-methyl-4-triazolopyridines, one or more isocytosines, one or more isoguanines, one or more inosines, one or more 7,8-dimethylalloxazines, one or more 6-dihydrothymines, one or more 5,6-dihydrouracils, one or more 4-methyl-indoles, ethenoadenines, or a combination thereof.

In some cases, the one or more analytes may comprise a nucleic acid with one or more genetic aberrations. The one or more genetic aberration may comprise one or more insertions, one or more deletions, one or more single nucleotide polymorphisms, one or more single nucleotide variations, one or more copy number variations, or a combination thereof.

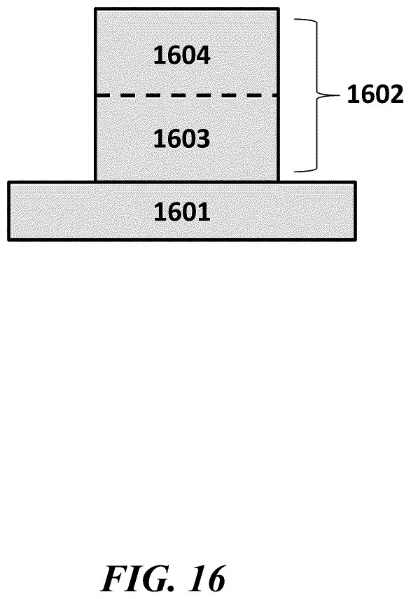

The methods described herein comprise contacting one or more analytes of a sample with one or more binding moieties. A binding moiety of the one or more binding moieties may comprise a probe that couples to the analyte (see FIG. 16). The binding moiety (1602) may comprise a probe (1603) and/or a sequence (1604). The probe (1603) may couple to or bind to the analyte (1601). The probe of the binding moiety of the one or more binding moieties may comprise a polypeptide. The polypeptide of the probe of the binding moiety may comprise an antibody, a nanobody, an antibody fragment, or combination thereof. For example, the analyte may be a protein target and/or the binding moiety may comprise an antibody probe. In some cases, the probe of the binding moiety may comprise an antibody probe and a sequence comprising a nucleic acid coupled thereto (e.g. an oligonucleotide). The oligonucleotide of the sequence coupled to the antibody probe may be single-stranded, double-stranded, or a combination thereof.

The one or more binding moieties may comprise one or more modifications. In some cases, the one or more binding moieties may comprise one or more padlock probes. The one or more padlock probes may comprise one or more phosphorylation modifications. The one or more phosphorylation may be located at a 3′ end of a binding moiety of the one or more binding moieties. The one or more phosphorylation may be located at a 5′ end of a binding moiety of the one or more binding moieties. In some cases, 5′ end of a binding moiety may be ligated to the 3′ end of a binding moiety. In some embodiments, the probe of the one or more probes of the one or more binding moieties that couples to the one or more analytes may comprise one or more nucleic acids. For example, the probe may comprise a DNA that couples to an exogenous RNA analyte within the sample. In some cases, the one or more nucleic acid of the probe may hybridize to at least a portion of a sequence of the analyte. For example, the nucleic acid of the probe may comprise an oligonucleotide that comprises a sequence that is the complement or reverse complement of a portion of an mRNA within the sample. In some cases, the nucleic acid of the probe may comprise DNA, RNA, or a combination thereof. The nucleic acid of the probe may comprise one or more modifications. The one or more modifications of the nucleic acid of the probe may comprise one or more phosphorylation groups, one or more methyl groups, one or more fluorescent dyes, one or more reactive chemical moieties, or a combination thereof. The nucleic acid of the probe may comprise an aptamer. The nucleic acids of the probe may comprise one or more nucleotides. The one of more nucleotides of the nucleic acid of the probe may comprise one or more natural nucleotides, one or more non-natural nucleotides, or a combination thereof. The one of more nucleotides of the nucleic acid of the probe may comprise one or more adenines, one or more guanines, one or more thymines, one or more cytosines, one or more uracils, one or more xanthines, one or more hypoxanthines, one or more 8-azapurines, one or more purines substituted at the 8 position with methyl or bromine, 9-oxo-N6-methyladenine, one or more 2-aminoadenines, one or more 7-deazaxanthines, one or more 7-deazaguanines, one or more 7-deaza-adenines, one or more N4-ethanocytosines, 2,6-diaminopurines, one or more N6-ethano-2,6-diaminopurines, one or more 5-methylcytosines, one or more 5-(C3-C6)-alkynylcytosines, one or more 5-alkynyluracils, one or more 5-fluorouracils, one or more 5-bromouracils, one or more thiouracils, one or more pseudoisocytosines, one or more 2-hydroxy-5-methyl-4-triazolopyridines, one or more isocytosines, one or more isoguanines, one or more inosines, one or more 7,8-dimethylalloxazines, one or more 6-dihydrothymines, one or more 5,6-dihydrouracils, one or more 4-methyl-indoles, ethenoadenines, or a combination thereof.

In some cases, the probe of the binding moiety of the one or more binding moieties that couples to one or more analytes may comprise one or more analyte-binding regions. The one or more analyte binding regions of the probe may couple to, hybridize to, associate with, or otherwise couple or bind to the one or more analytes. For example, the probe may comprise an antibody that couples to a protein, and/or the antibody may comprise an analyte-binding region that comprises an epitope that recognizes the protein. As another example, the probe may comprise a nucleic acid that comprises a sequence that hybridizes to an mRNA within the sample. The one or more analyte binding regions may comprise one or more nucleic acid sequences with a variety of lengths. The length of an analyte binding region of the one or more analyte binding regions of the probe of the binding moiety of the one or more binding moieties may comprise about 1 to about 300 nucleotides, about 2 to about 250 nucleotides, about 3 to about 200 nucleotides, about 4 to about 150 nucleotides, about 5 to about 100 nucleotides, about 6 to about 95 nucleotides, about 7 to about 90 nucleotides, about 8 to about 85 nucleotides, about 9 to about 80 nucleotides, about 10 to about 75 nucleotides, about 11 to about 70 nucleotides, about 12 to about 65 nucleotides, about 13 to about 60 nucleotides, about 14 to about 55 nucleotides, about 15 to about 50 nucleotides, about 16 to about 45 nucleotides, about 17 to about 40 nucleotides, about 18 to about 35 nucleotides, about 19 to about 30 nucleotides, about 20 to about 25 nucleotides, at least about, 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 150 nucleotides, at least about 200 nucleotides, at least about 250 nucleotides, at least about 300 nucleotides. At most about, 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, at most about 20 nucleotides, at most about 25 nucleotides, at most about 30 nucleotides, at most about 35 nucleotides, at most about 40 nucleotides, at most about 45 nucleotides, at most about 50 nucleotides, at most about 55 nucleotides, at most about 60 nucleotides, at most about 65 nucleotides, at most about 70 nucleotides, at most about 75 nucleotides, at most about 80 nucleotides, at most about 85 nucleotides, at most about 90 nucleotides, at most about 95 nucleotides, at most about 100 nucleotides, at most about 150 nucleotides, at most about 200 nucleotides, at most about 250 nucleotides, or at most about 300 nucleotides.

In some aspects, a binding moiety of the one or more binding moieties may comprise a sequence. The sequence of the binding moiety may comprise one or more subsequence or one or more pluralities of subsequences. For example, the sequence of the binding moiety may comprise 10 subsequences. In some cases, the sequence of the binding moiety may comprise at least about 1 subsequence, at least about 2 subsequences, at least about 3 subsequences, at least about 4 subsequences, at least about 5 subsequences, at least about 6 subsequences, at least about 7 subsequences, at least about 8 subsequences, at least about 9 subsequences, at least about 10 subsequences, at least about 11 subsequences, at least about 12 subsequences, at least about 13 subsequences, at least about 14 subsequences, at least about 15 subsequences, at least about 16 subsequences, at least about 17 subsequences, at least about 18 subsequences, at least about 19 subsequences, at least about 20 subsequences, at least about 25 subsequences, at least about 30 subsequences, at least about 35 subsequences, at least about 40 subsequences, at least about 45 subsequences, at least about 50 subsequences, at least about 55 subsequences, at least about 60 subsequences, at least about 65 subsequences, at least about 70 subsequences, at least about 75 subsequences, at least about 80 subsequences, at least about 85 subsequences, at least about 90 subsequences, at least about 95 subsequences, at least about 100 subsequences, or more subsequences. In some cases, the sequence of the binding moiety may comprise at most about 1 subsequence, at most about 2 subsequences, at most about 3 subsequences, at most about 4 subsequences, at most about 5 subsequences, at most about 6 subsequences, at most about 7 subsequences, at most about 8 subsequences, at most about 9 subsequences, at most about 10 subsequences, at most about 11 subsequences, at most about 12 subsequences, at most about 13 subsequences, at most about 14 subsequences, at most about 15 subsequences, at most about 16 subsequences, at most about 17 subsequences, at most about 18 subsequences, at most about 19 subsequences, at most about 20 subsequences, at most about 25 subsequences, at most about 30 subsequences, at most about 35 subsequences, at most about 40 subsequences, at most about 45 subsequences, at most about 50 subsequences, at most about 55 subsequences, at most about 60 subsequences, at most about 65 subsequences, at most about 70 subsequences, at most about 75 subsequences, at most about 80 subsequences, at most about 85 subsequences, at most about 90 subsequences, at most about 95 subsequences, at most about 100 subsequences, or fewer subsequences.

In some cases, a subsequence of the one or more pluralities of subsequences of the sequence of the binding moiety may comprise a nucleic acid sequence. In some cases, each of the subsequences of the one or more pluralities of subsequences may comprise one or more nucleotides. The one or more subsequences may have a length of at least about 1 nucleotide, at least about 2 nucleotides, 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, or more nucleotides. The one or more subsequences may have a length of at most about 1 nucleotide, at most about 2 nucleotides, 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, at most about 20 nucleotides, or fewer nucleotides. In some cases, each subsequence of the one or more pluralities of subsequence may comprise a portion of a nucleic acid. In some cases, each subsequence may be covalently attached to each other. In some cases, each subsequence of the one or more pluralities of subsequences may be covalently attached through phosphodiester bonds. The one or more detection probes described herein may couple or bind to one or more subsequences of the one or more pluralities of subsequences. In some cases, one or more detection probes described herein may couple or bind to a complement or derivative (e.g. a complement or a reverse complement) of one or more subsequences of the one or more pluralities of subsequences. For example, a binding moiety may comprise an antibody probe that is bound to a sequence comprising an oligonucleotide. The oligonucleotide of the sequence of the binding moiety may comprise one or more pluralities of subsequences, wherein a subsequence or complement or reverse complement thereof of the one or more pluralities of subsequences may comprise two nucleotides of the oligonucleotide. A detection probe may couple or bind to the subsequence of the sequence of the binding moiety via nucleic acid hybridization. In some cases, a binder may couple or bind to the oligonucleotide of the sequence of the binding moiety. The binder may comprise one or more subsequences or complement or reverse complements thereof.

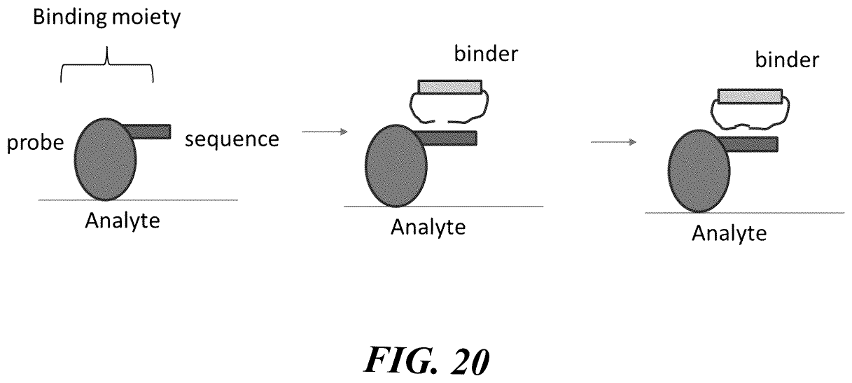

In some cases, a binding moiety of the present disclosure may comprise a sequence and the sequence of the binding moiety may couple or bind to a binder. For example, the binding moiety may comprise a sequence that comprises nucleic acid, and the binder may comprise a nucleic acid that hybridize to the sequence. The binder may comprise s single-stranded nucleic acid, a double stranded nucleic acid, or a combination thereof. In some cases, the binder may comprise a padlock probe comprising at least two binding sites that couple or bind to the sequence (See FIG. 20). For example, the binder may comprise a padlock probe nucleic acid that comprises a binding site at the 3′ end of the padlock probe and/or a binding site at the 5′ end of the padlock probe and/or the binding site at 3′ end of the padlock probe and/or the binding site at the 5′ end of the padlock probe may couple or bind to (e.g. hybridize to) the sequence of the binding moiety. The padlock probe of the binder may couple to (e.g. hybridize to) the sequence of the binding moiety. Upon coupling to the sequence, 5′ end of the padlock probe may be ligated to the 3′ end of the padlock probe (e.g. using a ligase). The binder may comprise one or more pluralities of subsequences. For example, the probe may be a padlock probe comprising one or more pluralities of subsequences. The one or more pluralities of subsequences or complement or reverse complements thereof of the binder may bind to or couple to one or more detection probes. In some cases, the one or more pluralities of subsequences or complements or reverse complements thereof may be amplified to generate a complement or a reverse complement of the one or more pluralities of subsequences or complements or reverse complements thereof. For example, the binder may be ligated to form a circular nucleic acid. The circular nucleic acid may be amplified using rolling circle amplification to form one or more amplicons. The one or more amplicons may comprise one or more pluralities of subsequences or complements or reverse complements thereof.

The methods described herein may comprise contacting the one or more analytes of the sample with one or more binding moieties. The contacting of the one or more analytes with the one or more binding moieties may comprise conditions that promote binding or coupling of the one or more binding moieties to the one or more analytes. For example, the contacting may result in a probe of a binding moiety of the one or more binding moieties to couple to or bind to an analyte of the one or more analytes. The conditions that promote binding between the one or more binding moieties and the one or more analytes may comprise incubating the sample with a reaction mixture. The incubating may comprise incubating the sample for a period of time. The period of time may be at least about 5 minutes, at least about 10 minutes, at least about 15 minutes, at least about 20 minutes, at least about 25 minutes, at least about 30 minutes, at least about 40 minutes, at least about 45 minutes, at least about 50 minutes, at least about 55 minutes, at least about 60 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 1 day, at least about 2 days, at least about 3 days at least about 4 days or longer. The length of time may be at most about 5 minutes, at most about 10 minutes, at most about 15 minutes, at most about 20 minutes, at most about 25 minutes, at most about 30 minutes, at most about 40 minutes, at most about 45 minutes, at most about 50 minutes, at most about 55 minutes, at most about 60 minutes, at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 hours, at most about 13 hours, at most about 14 hours, at most about 15 hours, at most about 16 hours, at most about 17 hours, at most about 18 hours, at most about 19 hours, at most about 20 hours, at most about 21 hours, at most about 22 hours, at most about 23 hours, at most about 24 hours, at most about 1 day, at most about 2 days, at most about 3 days at most about 4 days or less. The length of time may be about 5 minutes-24 hours, about 10 minutes-23 hours, about 15 minutes-22 hours, about 20 minutes-21 hours, about 25 minutes-20 hours, about 30 minutes-19 hours, about 40 minutes-18 hours, about 45 minutes-17 hours, about 50 minutes-16 hours, about 55 minutes-15 hours, about 60 minutes-14 hours, about 1 hour-13 hours, about 2 hours-12 hours, about 3 hours-11 hours, about 4 hours-10 hours, about 5 hours-9 hours, or about 6 hours-8 hours. The incubating may comprise incubating the sample at one or more temperatures. The one or more temperature may be at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., at least about 71° C., at least about 72° C., at least about 73° C., at least about 74° C., at least about 75° C., at least about 76° C., at least about 77° C., at least about 78° C., at least about 79° C., at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., or higher. The one or more temperature may be at most about 4° C., at most about 5° C., at most about 6° C., at most about 7° C., at most about 8° C., at most about 9° C., at most about 10° C., at most about 11° C., at most about 12° C., at most about 13° C., at most about 14° C., at most about 15° C., at most about 16° C., at most about 17° C., at most about 18° C., at most about 19° C., at most about 20° C., at most about 21° C., at most about 22° C., at most about 23° C., at most about 24° C., at most about 25° C., at most about 26° C., at most about 27° C., at most about 28° C., at most about 29° C., at most about 30° C., at most about 31° C., at most about 32° C., at most about 33° C., at most about 34° C., at most about 35° C., at most about 36° C., at most about 37° C., at most about 38° C., at most about 39° C., at most about 40° C., at most about 41° C., at most about 42° C., at most about 43° C., at most about 44° C., at most about 45° C., at most about 46° C., at most about 47° C., at most about 48° C., at most about 49° C., at most about 50° C., at most about 51° C., at most about 52° C., at most about 53° C., at most about 54° C., at most about 55° C., at most about 56° C., at most about 57° C., at most about 58° C., at most about 59° C., at most about 60° C., at most about 61° C., at most about 62° C., at most about 63° C., at most about 64° C., at most about 65° C., at most about 66° C., at most about 67° C., at most about 68° C., at most about 69° C., at most about 70° C., at most about 71° C., at most about 72° C., at most about 73° C., at most about 74° C., at most about 75° C., at most about 76° C., at most about 77° C., at most about 78° C., at most about 79° C., at most about 80° C., at most about 81° C., at most about 82° C., at most about 83° C., at most about 84° C., at most about 85° C., at most about 86° C., at most about 87° C., at most about 88° C., at most about 89° C., at most about 90° C., at most about 91° C., at most about 92° C., at most about 93° C., at most about 94° C., at most about 95° C., or lower. The one or more temperatures may be about 4-95° C., about 5-94° C., about 6-93° C., about 7-92° C., about 8-91° C., about 9-90° C., about 10-89° C., about 11-88° C., about 12-87° C., about 13-86° C., about 14-85° C., about 15-84° C., about 16-83° C., about 17-82° C., about 18-81° C., about 19-80° C., about 20-79° C., about 21-78° C., about 22-77° C., about 23-76° C., about 24-75° C., about 25-74° C., about 26-73° C., about 27-72° C., about 28-71° C., about 29-70° C., about 30-69° C., about 31-68° C., about 32-67° C., about 33-66° C., about 34-65° C., about 35-64° C., about 36-63° C., about 37-62° C., about 38-61° C., about 39-60° C., about 40-59° C., about 41-58° C., about 42-57° C., about 43-56° C., about 44-55° C., about 45-54° C., about 46-53° C., about 47-52° C., about 48-51° C., or about 49-50° C.

The reaction mixture to promote coupling of one or more binding moieties to one or more analytes may comprise one or more buffers. The one or more buffers of the reaction mixture may comprise MES (4-Morpholineethanesulfonic acid), Bis-Tris (Bis(2-hydroxyethyl)amino-tris(hydroxymethyl)methane), ADA, ACES, PIPES, MOSO, Bis-Tris Propane, BES, MOPS, TES, HEPES, DIPSO, MOBS, TAPSO, Tris, HEPPSO, POPSO, TEA, EPPS, Tricine, Gly-Gly, Bicine, HEPBS, TAPS, AMPD, TABS, AMPSO, CHES, CAPSO, AMP, CAPS, CAPS, Phosphate buffered saline, or a combination thereof. The reaction mixture to promote coupling of one or more binding moieties to one or more analytes may comprise one or more salts. The one or more salts of the reaction mixture may comprise NaCl, CaCl₂, MgCl₂, or a combination thereof. The reaction mixture to promote coupling of one or more binding moieties to one or more analytes may comprise one or more detergents. The one or more detergents of the reaction mixture may comprise SDS, Triton X-100, CHAPS, NP-40, Tween-20, Digitonin, or a combination thereof. The reaction mixture to promote coupling of one or more binding moieties to one or more analytes may comprise one or more solvents. The one or more solvents of the reaction mixture may comprise methanol, ethanol, ethyl acetate, DMSO, acetonitriles, water, or a combination thereof. The one or more chaotropic agents of the ligation reaction conditions may comprise DMSO, formamide, urea, thiourea, 2-propanol, guanidinium chloride, n-butanol, or a combination thereof. The reaction mixture to promote coupling of one or more binding moieties to one or more analytes may comprise a pH. The pH of the reaction mixture may be at least about 2, at least about 2.4, at least about 2.6, at least about 2.8, at least about 3, at least about 3.2, at least about 3.4, at least about 3.6, at least about 3.8, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, at least about 9, at least about 9.2, at least about 9.4, at least about 9.6, at least about 9.8, at least about 10, at least about 10.2, at least about 10.4, at least about 10.6, at least about 10.8, at least about 11, at least about 11.2, at least about 11.4, at least about 11.6, at least about 11.8, at least about 12, at most about 2, at most about 2.4, at most about 2.6, at most about 2.8, at most about 3, at most about 3.2, at most about 3.4, at most about 3.6, at most about 3.8, at most about 4, at most about 4.1, at most about 4.2, at most about 4.3, at most about 4.4, at most about 4.5, at most about 4.6, at most about 4.7, at most about 4.8, at most about 4.9, at most about 5, at most about 5.1, at most about 5.2, at most about 5.3, at most about 5.4, at most about 5.5, at most about 5.6, at most about 5.7, at most about 5.8, at most about 5.9, at most about 6, at most about 6.1, at most about 6.2, at most about 6.3, at most about 6.4, at most about 6.5, at most about 6.6, at most about 6.7, at most about 6.8, at most about 6.9, at most about 7, at most about 7.1, at most about 7.2, at most about 7.3, at most about 7.4, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 9.2, at most about 9.4, at most about 9.6, at most about 9.8, at most about 10, at most about 10.2, at most about 10.4, at most about 10.6, at most about 10.8, at most about 11, at most about 11.2, at most about 11.4, at most about 11.6, at most about 11.8, or at most about 12.

Contacting the one or more analytes with one or more binding moieties under conditions to promote coupling of the one or more binding moieties to the one or more analytes may result in the one or more binding moieties coupling to the one or more analytes. In some cases, the one or more binding moieties may comprise one or more nucleic acid probes. The one or more nucleic acid probes of the one or more binding moieties may hybridize to the one or more analytes under conditions to promote coupling (e.g. in the presence of the reaction mixture to promote coupling). In some cases, the one or more binding moieties may comprise one or more polypeptide probes (e.g. one or more antibodies). The one or more polypeptide probe of the one or more biding moieties may bind to or couple to the one or more analytes under conditions to promote coupling (e.g. the reaction mixture to promote coupling).

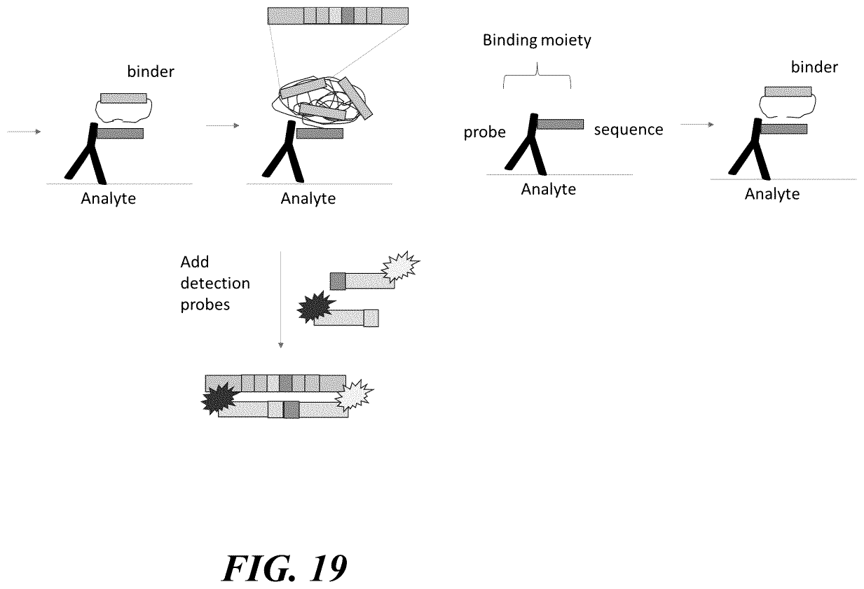

In some cases, the one or more detection probes (e.g. the first detection probe, the second detection probe, or a combination thereof) may couple or bind to one or more subsequences or complement or reverse complement of the one or more pluralities of subsequences or complements or reverse complements thereof. An example of a method that uses a binder is shown in FIG. 19. As shown in FIG. 19, an analyte may be bound by a binding moiety that comprises a probe and one or more sequences. In some cases, the binding moiety may comprise a probe and one or more sequences (e.g. pre-determined sequences). The one or more pre-determined subsequences may comprise a nucleic acid sequence that correlates to (e.g. corresponds to) the analyte. For example, detecting the one or more pre-determined subsequences may enable identification of the analyte. In some cases, the one or more pre-determined subsequences may correlate to the analyte (e.g. correspond to the analyte) by enabling a binder to couple or bind to the one or more pre-determined subsequences. The binder may comprise one or more pluralities of subsequences. The one or more pluralities of subsequences of the binder may be detected. The probe may comprise an antibody that couples or binds to the analyte. A binder may couple or bind to the sequence (e.g. pre-determined sequence) attached to the antibody. The binder may comprise one or more pluralities of subsequences. The binder may comprise a padlock probe. The padlock probe may be ligated to form a circular nucleic acid. The circular nucleic acid may be amplified (e.g. using rolling circle amplification) to generate one or more amplicons. The one or more amplicons may comprise one or more pluralities of subsequences or complements or reverse complements thereof. Detection probes may be added to the sample comprising the one or more amplicons. A first detection probe of the detection probes may couple or bind to a first subsequence or complement or reverse complement thereof of the one or more subsequences or complements or reverse complements thereof. The first detection probe may comprise a first detection moiety. A second detection probe of the detection probes may couple or bind to a second subsequence or complement or reverse complement thereof of the one or more pluralities of subsequences or complements or reverse complements thereof. The second detection probe may comprise a second detection moiety. The first detection probe may be ligated to the second detection probe. The first detection moiety of the first detection probe and/or the second detection moiety of the second detection probe may be detected. The detection of the first detection moiety and/or the second detection moiety corresponding to the first subsequence or complement or reverse complement thereof and/or the second subsequence or complement or reverse complement thereof may be used to identify the analyte.

The methods described herein may comprise contacting the sample with one or more binders under conditions to promote the one or more binders to bind to one or more binding moieties. In some cases, the one or more binders may couple with a sequence of a binding moiety of the one or more binding moieties. In some cases, the one or more binders may comprise nucleic acid. Coupling of the nucleic acid of the one or more binders to the one or more binding moieties may comprise nucleic acid hybridization between the one or more binders and the one or more binding moieties.

The conditions that promote binding between the one or more binders to bind to one or more binding moieties may comprise incubating the sample with a reaction mixture. The incubating may comprise incubating the sample for a period of time. The period of time may be at least about 5 minutes, at least about 10 minutes, at least about 15 minutes, at least about 20 minutes, at least about 25 minutes, at least about 30 minutes, at least about 40 minutes, at least about 45 minutes, at least about 50 minutes, at least about 55 minutes, at least about 60 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 1 day, at least about 2 days, at least about 3 days at least about 4 days or longer. The length of time may be at most about 5 minutes, at most about 10 minutes, at most about 15 minutes, at most about 20 minutes, at most about 25 minutes, at most about 30 minutes, at most about 40 minutes, at most about 45 minutes, at most about 50 minutes, at most about 55 minutes, at most about 60 minutes, at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 hours, at most about 13 hours, at most about 14 hours, at most about 15 hours, at most about 16 hours, at most about 17 hours, at most about 18 hours, at most about 19 hours, at most about 20 hours, at most about 21 hours, at most about 22 hours, at most about 23 hours, at most about 24 hours, at most about 1 day, at most about 2 days, at most about 3 days at most about 4 days or less. The length of time may be about 5 minutes-24 hours, about 10 minutes-23 hours, about 15 minutes-22 hours, about 20 minutes-21 hours, about 25 minutes-20 hours, about 30 minutes-19 hours, about 40 minutes-18 hours, about 45 minutes-17 hours, about 50 minutes-16 hours, about 55 minutes-15 hours, about 60 minutes-14 hours, about 1 hour-13 hours, about 2 hours-12 hours, about 3 hours-11 hours, about 4 hours-10 hours, about 5 hours-9 hours, or about 6 hours-8 hours. The incubating may comprise incubating the sample at one or more temperatures. The one or more temperature may be at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., at least about 71° C., at least about 72° C., at least about 73° C., at least about 74° C., at least about 75° C., at least about 76° C., at least about 77° C., at least about 78° C., at least about 79° C., at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., or higher. The one or more temperature may be at most about 4° C., at most about 5° C., at most about 6° C., at most about 7° C., at most about 8° C., at most about 9° C., at most about 10° C., at most about 11° C., at most about 12° C., at most about 13° C., at most about 14° C., at most about 15° C., at most about 16° C., at most about 17° C., at most about 18° C., at most about 19° C., at most about 20° C., at most about 21° C., at most about 22° C., at most about 23° C., at most about 24° C., at most about 25° C., at most about 26° C., at most about 27° C., at most about 28° C., at most about 29° C., at most about 30° C., at most about 31° C., at most about 32° C., at most about 33° C., at most about 34° C., at most about 35° C., at most about 36° C., at most about 37° C., at most about 38° C., at most about 39° C., at most about 40° C., at most about 41° C., at most about 42° C., at most about 43° C., at most about 44° C., at most about 45° C., at most about 46° C., at most about 47° C., at most about 48° C., at most about 49° C., at most about 50° C., at most about 51° C., at most about 52° C., at most about 53° C., at most about 54° C., at most about 55° C., at most about 56° C., at most about 57° C., at most about 58° C., at most about 59° C., at most about 60° C., at most about 61° C., at most about 62° C., at most about 63° C., at most about 64° C., at most about 65° C., at most about 66° C., at most about 67° C., at most about 68° C., at most about 69° C., at most about 70° C., at most about 71° C., at most about 72° C., at most about 73° C., at most about 74° C., at most about 75° C., at most about 76° C., at most about 77° C., at most about 78° C., at most about 79° C., at most about 80° C., at most about 81° C., at most about 82° C., at most about 83° C., at most about 84° C., at most about 85° C., at most about 86° C., at most about 87° C., at most about 88° C., at most about 89° C., at most about 90° C., at most about 91° C., at most about 92° C., at most about 93° C., at most about 94° C., at most about 95° C., or lower. The one or more temperatures may be about 4-95° C., about 5-94° C., about 6-93° C., about 7-92° C., about 8-91° C., about 9-90° C., about 10-89° C., about 11-88° C., about 12-87° C., about 13-86° C., about 14-85° C., about 15-84° C., about 16-83° C., about 17-82° C., about 18-81° C., about 19-80° C., about 20-79° C., about 21-78° C., about 22-77° C., about 23-76° C., about 24-75° C., about 25-74° C., about 26-73° C., about 27-72° C., about 28-71° C., about 29-70° C., about 30-69° C., about 31-68° C., about 32-67° C., about 33-66° C., about 34-65° C., about 35-64° C., about 36-63° C., about 37-62° C., about 38-61° C., about 39-60° C., about 40-59° C., about 41-58° C., about 42-57° C., about 43-56° C., about 44-55° C., about 45-54° C., about 46-53° C., about 47-52° C., about 48-51° C., or about 49-50° C.

The reaction mixture to promote coupling of the one or more binders to bind to one or more binding moieties may comprise one or more buffers. The one or more buffers of the reaction mixture may comprise MES (4-Morpholineethanesulfonic acid), Bis-Tris (Bis(2-hydroxyethyl)amino-tris(hydroxymethyl)methane), ADA, ACES, PIPES, MOSO, Bis-Tris Propane, BES, MOPS, TES, HEPES, DIPSO, MOBS, TAPSO, Tris, HEPPSO, POPSO, TEA, EPPS, Tricine, Gly-Gly, Bicine, HEPBS, TAPS, AMPD, TABS, AMPSO, CHES, CAPSO, AMP, CAPS, CAPS, Phosphate buffered saline, or a combination thereof. The reaction mixture to promote coupling of the one or more binders to bind to one or more binding moieties may comprise one or more salts. The one or more salts of the reaction mixture may comprise NaCl, CaCl₂, MgCl₂, or a combination thereof. The reaction mixture to promote coupling of the one or more binders to bind to one or more binding moieties may comprise one or more detergents. The one or more detergents of the reaction mixture may comprise SDS, Triton X-100, CHAPS, NP-40, Tween-20, Digitonin, or a combination thereof. The reaction mixture to promote coupling of the one or more binders to bind to one or more binding moieties may comprise one or more solvents. The one or more solvents of the reaction mixture may comprise methanol, ethanol, ethyl acetate, DMSO, acetonitriles, water, or a combination thereof. The one or more chaotropic agents of the ligation reaction conditions may comprise DMSO, formamide, urea, thiourea, 2-propanol, guanidinium chloride, n-butanol, or a combination thereof. The reaction mixture to promote coupling of the one or more binders to bind to one or more binding moieties may comprise a pH. The pH of the reaction mixture may be at least about 2, at least about 2.4, at least about 2.6, at least about 2.8, at least about 3, at least about 3.2, at least about 3.4, at least about 3.6, at least about 3.8, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, at least about 9, at least about 9.2, at least about 9.4, at least about 9.6, at least about 9.8, at least about 10, at least about 10.2, at least about 10.4, at least about 10.6, at least about 10.8, at least about 11, at least about 11.2, at least about 11.4, at least about 11.6, at least about 11.8, at least about 12, at most about 2, at most about 2.4, at most about 2.6, at most about 2.8, at most about 3, at most about 3.2, at most about 3.4, at most about 3.6, at most about 3.8, at most about 4, at most about 4.1, at most about 4.2, at most about 4.3, at most about 4.4, at most about 4.5, at most about 4.6, at most about 4.7, at most about 4.8, at most about 4.9, at most about 5, at most about 5.1, at most about 5.2, at most about 5.3, at most about 5.4, at most about 5.5, at most about 5.6, at most about 5.7, at most about 5.8, at most about 5.9, at most about 6, at most about 6.1, at most about 6.2, at most about 6.3, at most about 6.4, at most about 6.5, at most about 6.6, at most about 6.7, at most about 6.8, at most about 6.9, at most about 7, at most about 7.1, at most about 7.2, at most about 7.3, at most about 7.4, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 9.2, at most about 9.4, at most about 9.6, at most about 9.8, at most about 10, at most about 10.2, at most about 10.4, at most about 10.6, at most about 10.8, at most about 11, at most about 11.2, at most about 11.4, at most about 11.6, at most about 11.8, or at most about 12.

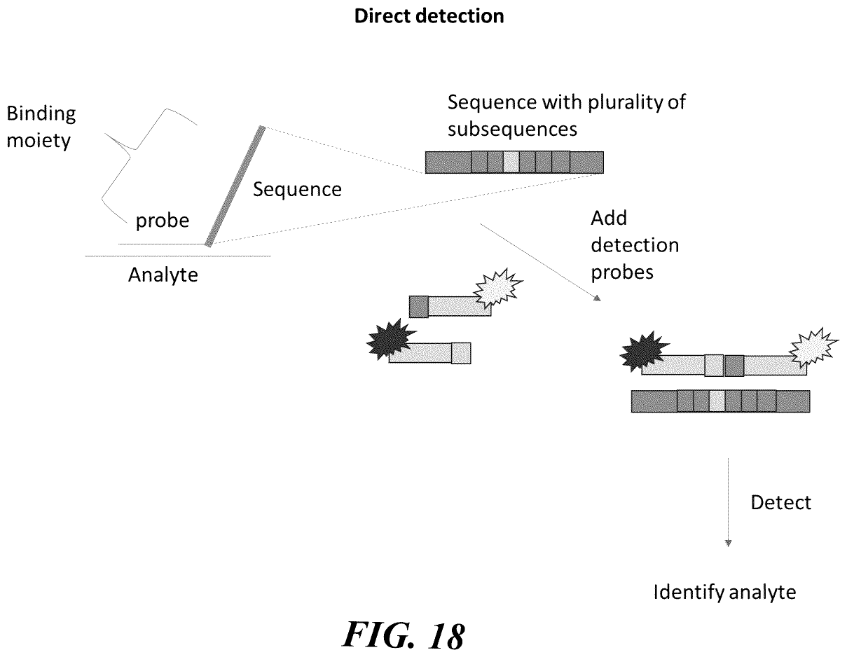

In some cases, the methods described herein may comprise direct detection of one or more subsequences. An example of direct detection of one or more subsequences is shown in FIG. 18. A binding moiety may be added to a sample. The sample may comprise one or more probes and/or one or more sequences. The one or more probes may couple to or bind to the analyte. For example, the one or more probes may comprise nucleic acid and may hybridize to the analyte. A sequence of the one or more sequences of the binding moiety may comprise one or more pluralities of subsequences or complements or reverse complements thereof. The one or more pluralities of subsequences or complements or reverse complements thereof may correlate with the analyte. Detection probes may be added to the sample. A first detection probe of the detection probes may couple or bind to a first subsequence or complement or reverse complement thereof of the one or more subsequences or complements or reverse complements thereof of the sequence of the binding moiety. The first detection probe may comprise a first detection moiety. A second detection probe of the detection probes may couple or bind to a second subsequence or complement or reverse complement thereof of the one or more pluralities of subsequences. The second detection probe may comprise a second detection moiety. The first detection probe may be ligated to the second detection probe. The first detection moiety of the first detection probe and/or the second detection moiety of the second detection probe may be detected. The detection of the first detection moiety and/or the second detection moiety corresponding to the first subsequence or complement or reverse complement thereof and/or the second subsequence or complement or reverse complement thereof may be used to identify the analyte.

In some aspects, a subsequence of the one or more pluralities of subsequences (e.g. the first subsequence and/or the second subsequence) of a sequence or of a probe may have a variety of lengths. In some cases, a subsequence of the one or more pluralities of subsequences may comprise one or more nucleotides. In some cases, a subsequence of the one or more pluralities of subsequences may comprise two or more nucleotides. In some cases, a subsequence of the one or more pluralities of subsequences may comprise three or more nucleotides. In some cases, a subsequence of the one or more pluralities of subsequences may comprise about 1 to about 20 nucleotides, about 2 to about 19 nucleotides, about 3 to about 18 nucleotides, about 4 to about 17 nucleotides, about 5 to about 16 nucleotides, about 6 to about 15 nucleotides, about 7 to about 14 nucleotides, about 8 to about 13 nucleotides, about 9 to about 12 nucleotides, about 10 to about 11 nucleotides, at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at most about 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, or at most about 20 nucleotides. In some embodiments, each of the subsequences of the one or more pluralities of subsequences may have the same length. In some embodiments, each of the subsequences of the one or more pluralities of subsequences may have different lengths. In some embodiments, at least two of the subsequences of the one or more pluralities of subsequences have the same length. In some embodiments, at least two of the subsequences of the one or more pluralities of subsequences have different lengths (e.g. lengths that are not the same).

The first subsequence may comprise a variety of lengths. For example, the first subsequence may comprise one or more nucleotides. In some cases, the first subsequence may comprise nucleic acid and/or may comprise at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 25 nucleotides, at least about 30 nucleotides, or more nucleotides. In some cases, the first subsequence may comprise nucleic acid and/or may comprise at most about 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, at most about 20 nucleotides, at most about 25 nucleotides, at most about 30 nucleotides, or fewer nucleotides. In some cases, the first subsequence may comprise nucleic acid and/or may comprise about 1 to about 30 nucleotides, about 2 to about 25 nucleotides, about 3 to about 20 nucleotides, about 4 to about 19 nucleotides, about 5 to about 18 nucleotides, about 6 to about 17 nucleotides, about 7 to about 16 nucleotides, about 8 to about 15 nucleotides, about 9 to about 14 nucleotides, about 10 to about 13 nucleotides, or about 11 to about 12 nucleotides.

The second subsequence may comprise a variety of lengths. For example, the second subsequence may comprise one or more nucleotides. In some cases, the second subsequence may comprise nucleic acid and/or may comprise at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 25 nucleotides, at least about 30 nucleotides, or more nucleotides. In some cases, the second subsequence may comprise nucleic acid and/or may comprise at most about 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, at most about 20 nucleotides, at most about 25 nucleotides, at most about 30 nucleotides, or fewer nucleotides. In some cases, the second subsequence may comprise nucleic acid and/or may comprise about 1 to about 30 nucleotides, about 2 to about 25 nucleotides, about 3 to about 20 nucleotides, about 4 to about 19 nucleotides, about 5 to about 18 nucleotides, about 6 to about 17 nucleotides, about 7 to about 16 nucleotides, about 8 to about 15 nucleotides, about 9 to about 14 nucleotides, about 10 to about 13 nucleotides, or about 11 to about 12 nucleotides.

In some embodiments, the first subsequence and/or second subsequence may be part of a nucleic acid molecule. In some cases, the first subsequence and the second subsequence of the nucleic acid molecule may each comprise at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 25 nucleotides, at least about 30 nucleotides, or more nucleotides. In some cases, the first subsequence and/or the second subsequence of the nucleic acid molecule may each comprise at most about 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, at most about 20 nucleotides, at most about 25 nucleotides, at most about 30 nucleotides, or fewer nucleotides. In some cases, the first subsequence and/or the second subsequence of the nucleic acid molecule may each comprise about 1 to about 30 nucleotides, about 2 to about 25 nucleotides, about 3 to about 20 nucleotides, about 4 to about 19 nucleotides, about 5 to about 18 nucleotides, about 6 to about 17 nucleotides, about 7 to about 16 nucleotides, about 8 to about 15 nucleotides, about 9 to about 14 nucleotides, about 10 to about 13 nucleotides, or about 11 to about 12 nucleotides.

In some cases, the a first subsequence (e.g. the first subsequence) of the one or more pluralities of subsequences may be the same length as a second subsequence (e.g. the second subsequence) of the one or more pluralities of subsequences. For example, the first subsequence may be two or more nucleotides in length and/or the second subsequence may be two or more nucleotides in length. In some cases, the first subsequence may be a different length than the second subsequence (e.g. the first subsequence length does not equal the second subsequence length). For example, the first subsequence may be 2 nucleotides in length and/or the second subsequence may be one nucleotide in length.

In some aspects, one or more detection probes of the present disclosure (e.g. the first detection probe, the second detection probe, or a combination thereof) may comprise a nucleic acid. The one or more detection probes of the present disclosure may couple or bind to one or more subsequences of the one or more pluralities of subsequences, the complement or reverse complement of one or more subsequences of the one or more pluralities of subsequences, or a combination thereof. The binding or coupling of one or more detection probes may be a result of contacting the sample with the one or more detection probes. For example, a first detection probe may comprise an oligonucleotide. In some cases, a detection probe may comprise a nucleic acid that couples or binds to (e.g. hybridizes to) a subsequence (e.g. the first subsequence or the second subsequence). In some cases, a detection probe may comprise a nucleic acid that couples or binds to (e.g. hybridizes to) a complement or a reverse complement of a subsequence (e.g. a complement or a reverse complement of the first subsequence or a complement or a reverse complement of the second subsequence). A detection probe may couple or bind to sequence adjacent to a subsequence (e.g. a first subsequence or a second subsequence). In some cases, a detection probe may couple or bind to a subsequence and/or one or more sequences adjacent to the subsequence. For example, a detection probe may hybridize to a subsequence and a portion of the sequence adjacent to the subsequence. In some cases, the subsequence may comprise two or more nucleotides (e.g. AA) and the two or more nucleotides may be part of a nucleic acid with more than two nucleotides or more. The detection probe may couple or bind the nucleic acid including the two or more nucleotides of the subsequence (in this example ‘AA’) as well as a portion of the nucleic acid sequence adjacent to the subsequence. In some cases, the first detection probe may comprise a nucleic acid, wherein the nucleic acid may couple or bind to a region adjacent to the first subsequence. The region adjacent to the a subsequence (e.g. a portion of a nucleic acid next to a subsequence) may comprise about 1 to about 20 nucleotides, about 2 to about 19 nucleotides, about 3 to about 18 nucleotides, about 4 to about 17 nucleotides, about 5 to about 16 nucleotides, about 6 to about 15 nucleotides, about 7 to about 14 nucleotides, about 8 to about 13 nucleotides, about 9 to about 12 nucleotides, about 10 to about 11 nucleotides, at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at most about 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, or at most about 20 nucleotides.

In some cases, the second detection probe may comprise a nucleic acid. The nucleic acid of the second detection probe may couple or bind to the second subsequence. In some cases, the second detection probe may comprise a nucleic acid, wherein the nucleic acid may couple or bind to a region adjacent to the second subsequence. The region adjacent to the second subsequence may comprise about 1 to about 20 nucleotides, about 2 to about 19 nucleotides, about 3 to about 18 nucleotides, about 4 to about 17 nucleotides, about 5 to about 16 nucleotides, about 6 to about 15 nucleotides, about 7 to about 14 nucleotides, about 8 to about 13 nucleotides, about 9 to about 12 nucleotides, about 10 to about 11 nucleotides, at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at most about 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, or at most about 20 nucleotides.

The one or more detection probes as described herein may comprise one or more nucleotides that couple to one or more subsequences. In some cases, a detection probe may comprise one or more nucleotides at one end of the detection probe that couple to or bind to one or more subsequences. For example, a detection probe may comprise a 3′ terminal nucleotide that binds to or couples a subsequence of a sequence or complement or reverse complement thereof. The detection probe may comprise a 3′ terminal A (e.g. 5′-NNNNNA-3′) where the A binds to or couples to a T of the subsequence of the sequence or complement or reverse complement thereof and the N's represent different combinations of A, C, T, and G. In other examples, a detection probe may comprise a 5′ terminal nucleotide that binds to or couples a subsequence of a sequence or complement or reverse complement thereof. The detection probe may comprise a 5′ terminal A (e.g. 5′-ANNNNN-3′) where the A binds to or couples to a T of the subsequence of the sequence or complement or reverse complement thereof and the N's represent different combinations of A, C, T, and G. In some cases, the detection probe may comprise two or more nucleotides at one end of the detection probe that couples to or binds to a subsequence of a sequence or complement or reverse complement thereof. The detection probe may comprise a 3′ terminal AA (e.g. 5′-NNNNNAA-3′) where the AA binds to or couples to a TT of the subsequence of the sequence or complement or reverse complement thereof and the N's represent different combinations of A, C, T, and G. In other examples, a detection probe may comprise a 5′ terminal nucleotide that binds to or couples a subsequence of a sequence or complement or reverse complement thereof. The detection probe may comprise a 5′ terminal AA (e.g. 5′-AANNNNN-3′) where the AA binds to or couples to a TT of the subsequence of the sequence or complement or reverse complement thereof and the N's represent different combinations of A, C, T, and G. In some cases, a detection probe may comprise a nucleotide or nucleotides with a fixed position that is not at either 3′ or 5′ end. For example, the detection probe may comprise one or more nucleotides that couples to or binds to a subsequence of a sequence or complement or reverse complement thereof where the one or more nucleotides are internal within the detection probe. For example, the detection probe may comprise an internal A (e.g. 5′-NNNNNAN-3′) that binds to or couples to a T the subsequence of the sequence or complement or reverse complement thereof and the N's represent different combinations of A, C, T, and G.

In some aspects, the detection probe (e.g. a first detection probe or a second detection probe) may comprise a detection moiety (e.g. a first detection moiety or a second detection moiety). The detection moiety (e.g. the first detection moiety or the second detection moiety) of the detection probe may be attached to a 5′ end of the detection probe, 3′ end of the detection probe, an internal region of the detection probe, or a combination thereof. For example, a detection probe of the present disclosure may comprise an oligonucleotide with a detection moiety attached to 5′ end of the oligonucleotide of the detection probe. The detection moiety of the detection probe may comprise a fluorescent dye, a quantum dot, an enzyme, a linker, or a combination thereof. In some cases, the fluorescent dye may comprise phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), fluorescein and/or fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and/or rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and/or coumarin derivatives such as AMCA, AMCA-NHS, AMCA-sulfo-NHS, AMCA-HPDP, DCIA, AMCE-hydrazide, BODIPY and/or derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and/or derivatives such as Cascade Blue acetyl azide, Cascade Blue cadaverine, Cascade Blue ethyl enediamine, Cascade Blue hydrazide, Lucifer Yellow and/or derivatives such as Lucifer Yellow iodoacetamide, Lucifer Yellow CH, cyanine and/or derivatives such as indolium based cyanine dyes, benzo-indolium based cyanine dyes, pyridium based cyanine dyes, thiozolium based cyanine dyes, quinolinium based cyanine dyes, imidazolium based cyanine dyes, lanthanide chelates and/or derivatives such as BCPDA, TBP, TMT, BHHCT, BCOT, Europium chelates, Terbium chelates, DyLight dyes, Atto dyes, LightCycler Red dyes, CAL Flour dyes, JOE and/or derivatives thereof, Oregon Green dyes, WellRED dyes, IRD dyes, phycoerythrin, phycobilin dyes, or a combination thereof. In some cases, the enzyme may comprise firefly luciferase, Renilla luciferase, NADPH, beta-galactosidase, horseradish peroxidase, glucose oxidase, alkaline phosphatase, chloramphenicol acetyl transferase, urease, or a combination thereof. In some embodiments, the linker may comprise a succinimidyl-4-(N-maleimidomethyl)cyclohexane-1-carboxylate (SMCC) linker, a sulfo-SMCC linker, a succinimidyl-6-hydrazino-nicotinamide (S-HyNic) linker, an N-succinimidyl-4-formylbenzamide (S-4FB) linker, a bis-aryl hydrazone bond (from S-HyNic/S-4FB reaction), a zero-length peptide bond (between —COOH and/or —NH₂ directly on affinity molecule and/or nucleic acid), a triazole bond (from “click” reaction), a phosphodiester linkage, a phosphothioate linkage, or any combination thereof. In some cases, the first detection moiety is coupled to a 5′ end of a nucleic acid of the first detection probe. In some cases, the first detection moiety is coupled to a 3′ end of a nucleic acid of the first detection probe. In some cases, the first detection moiety is coupled to an internal nucleotide of a nucleic acid of the first detection probe. In some cases, the second detection moiety is coupled to a 5′ end of a nucleic acid of the second detection probe. In some cases, the second detection moiety is coupled to a 3′ end of a nucleic acid of the second detection probe. In some cases, the second detection moiety is coupled to an internal nucleotide of a nucleic acid of the second detection probe.

The methods described herein may comprise contacting the sample with one or more detection probes under conditions to promote the one or more detection probes to bind to one or more subsequences or complements or reverse complements thereof. In some cases, the one or more detection probes may couple with or hybridize to one or more subsequences or complements or reverse complements thereof of a sequence of a binding moiety. In some cases, the one or more detection probes may couple with or hybridize to one or more subsequences or complements or reverse complements thereof of a sequence of a binder. In some cases, the one or more detection probes may couple with or hybridize to one or more subsequences or complements or reverse complements thereof of a sequence or complement or reverse complement thereof of an amplification product of a binder (e.g. one or more amplicons). In some cases, the one or more detection probes may comprise nucleic acid. Coupling of the nucleic acid of the one or more detection probes to the one or more subsequences or complements or reverse complements thereof may comprise nucleic acid hybridization between the one or more detection probes and the one or more subsequences or complements or reverse complements thereof.

The conditions that promote binding between the one or more detection probes to bind to one or more subsequences or complements or reverse complements thereof may comprise incubating the sample with a reaction mixture. The incubating may comprise incubating the sample for a period of time. The period of time may be at least about 5 minutes, at least about 10 minutes, at least about 15 minutes, at least about 20 minutes, at least about 25 minutes, at least about 30 minutes, at least about 40 minutes, at least about 45 minutes, at least about 50 minutes, at least about 55 minutes, at least about 60 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 1 day, at least about 2 days, at least about 3 days at least about 4 days or longer. The length of time may be at most about 5 minutes, at most about 10 minutes, at most about 15 minutes, at most about 20 minutes, at most about 25 minutes, at most about 30 minutes, at most about 40 minutes, at most about 45 minutes, at most about 50 minutes, at most about 55 minutes, at most about 60 minutes, at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 hours, at most about 13 hours, at most about 14 hours, at most about 15 hours, at most about 16 hours, at most about 17 hours, at most about 18 hours, at most about 19 hours, at most about 20 hours, at most about 21 hours, at most about 22 hours, at most about 23 hours, at most about 24 hours, at most about 1 day, at most about 2 days, at most about 3 days at most about 4 days or less. The length of time may be about 5 minutes-24 hours, about 10 minutes-23 hours, about 15 minutes-22 hours, about 20 minutes-21 hours, about 25 minutes-20 hours, about 30 minutes-19 hours, about 40 minutes-18 hours, about 45 minutes-17 hours, about 50 minutes-16 hours, about 55 minutes-15 hours, about 60 minutes-14 hours, about 1 hour-13 hours, about 2 hours-12 hours, about 3 hours-11 hours, about 4 hours-10 hours, about 5 hours-9 hours, or about 6 hours-8 hours. The incubating may comprise incubating the sample at one or more temperatures. The one or more temperature may be at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., at least about 71° C., at least about 72° C., at least about 73° C., at least about 74° C., at least about 75° C., at least about 76° C., at least about 77° C., at least about 78° C., at least about 79° C., at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., or higher. The one or more temperature may be at most about 4° C., at most about 5° C., at most about 6° C., at most about 7° C., at most about 8° C., at most about 9° C., at most about 10° C., at most about 11° C., at most about 12° C., at most about 13° C., at most about 14° C., at most about 15° C., at most about 16° C., at most about 17° C., at most about 18° C., at most about 19° C., at most about 20° C., at most about 21° C., at most about 22° C., at most about 23° C., at most about 24° C., at most about 25° C., at most about 26° C., at most about 27° C., at most about 28° C., at most about 29° C., at most about 30° C., at most about 31° C., at most about 32° C., at most about 33° C., at most about 34° C., at most about 35° C., at most about 36° C., at most about 37° C., at most about 38° C., at most about 39° C., at most about 40° C., at most about 41° C., at most about 42° C., at most about 43° C., at most about 44° C., at most about 45° C., at most about 46° C., at most about 47° C., at most about 48° C., at most about 49° C., at most about 50° C., at most about 51° C., at most about 52° C., at most about 53° C., at most about 54° C., at most about 55° C., at most about 56° C., at most about 57° C., at most about 58° C., at most about 59° C., at most about 60° C., at most about 61° C., at most about 62° C., at most about 63° C., at most about 64° C., at most about 65° C., at most about 66° C., at most about 67° C., at most about 68° C., at most about 69° C., at most about 70° C., at most about 71° C., at most about 72° C., at most about 73° C., at most about 74° C., at most about 75° C., at most about 76° C., at most about 77° C., at most about 78° C., at most about 79° C., at most about 80° C., at most about 81° C., at most about 82° C., at most about 83° C., at most about 84° C., at most about 85° C., at most about 86° C., at most about 87° C., at most about 88° C., at most about 89° C., at most about 90° C., at most about 91° C., at most about 92° C., at most about 93° C., at most about 94° C., at most about 95° C., or lower. The one or more temperatures may be about 4-95° C., about 5-94° C., about 6-93° C., about 7-92° C., about 8-91° C., about 9-90° C., about 10-89° C., about 11-88° C., about 12-87° C., about 13-86° C., about 14-85° C., about 15-84° C., about 16-83° C., about 17-82° C., about 18-81° C., about 19-80° C., about 20-79° C., about 21-78° C., about 22-77° C., about 23-76° C., about 24-75° C., about 25-74° C., about 26-73° C., about 27-72° C., about 28-71° C., about 29-70° C., about 30-69° C., about 31-68° C., about 32-67° C., about 33-66° C., about 34-65° C., about 35-64° C., about 36-63° C., about 37-62° C., about 38-61° C., about 39-60° C., about 40-59° C., about 41-58° C., about 42-57° C., about 43-56° C., about 44-55° C., about 45-54° C., about 46-53° C., about 47-52° C., about 48-51° C., or about 49-50° C.

The reaction mixture to promote coupling of the one or more detection probes to bind to one or more subsequences or complements or reverse complements thereof may comprise one or more buffers. The one or more buffers of the reaction mixture may comprise MES (4-Morpholineethanesulfonic acid), Bis-Tris(Bis(2-hydroxyethyl)amino-tris(hydroxymethyl)methane), ADA, ACES, PIPES, MOSO, Bis-Tris Propane, BES, MOPS, TES, HEPES, DIPSO, MOBS, TAPSO, Tris, HEPPSO, POPSO, TEA, EPPS, Tricine, Gly-Gly, Bicine, HEPBS, TAPS, AMPD, TABS, AMPSO, CHES, CAPSO, AMP, CAPS, CAPS, Phosphate buffered saline, or a combination thereof. The reaction mixture to promote coupling the one or more detection probes to bind to one or more subsequences or complements or reverse complements thereof may comprise one or more salts. The one or more salts of the reaction mixture may comprise NaCl, CaCl₂, MgCl₂, or a combination thereof. The reaction mixture to promote coupling of the one or more detection probes to bind to one or more subsequences or complements or reverse complements thereof may comprise one or more detergents. The one or more detergents of the reaction mixture may comprise SDS, Triton X-100, CHAPS, NP-40, Tween-20, Digitonin, or a combination thereof. The reaction mixture to promote coupling of the one or more detection probes to bind to one or more subsequences or complements or reverse complements thereof may comprise one or more solvents. The one or more solvents of the reaction mixture may comprise methanol, ethanol, ethyl acetate, DMSO, acetonitrile, water, or a combination thereof. The one or more chaotropic agents of the ligation reaction conditions may comprise DMSO, formamide, urea, thiourea, 2-propanol, guanidinium chloride, n-butanol, or a combination thereof. The reaction mixture to promote coupling of the one or more detection probes to bind to one or more subsequences or complements or reverse complements thereof may comprise a pH. The pH of the reaction mixture may be at least about 2, at least about 2.4, at least about 2.6, at least about 2.8, at least about 3, at least about 3.2, at least about 3.4, at least about 3.6, at least about 3.8, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, at least about 9, at least about 9.2, at least about 9.4, at least about 9.6, at least about 9.8, at least about 10, at least about 10.2, at least about 10.4, at least about 10.6, at least about 10.8, at least about 11, at least about 11.2, at least about 11.4, at least about 11.6, at least about 11.8, at least about 12, at most about 2, at most about 2.4, at most about 2.6, at most about 2.8, at most about 3, at most about 3.2, at most about 3.4, at most about 3.6, at most about 3.8, at most about 4, at most about 4.1, at most about 4.2, at most about 4.3, at most about 4.4, at most about 4.5, at most about 4.6, at most about 4.7, at most about 4.8, at most about 4.9, at most about 5, at most about 5.1, at most about 5.2, at most about 5.3, at most about 5.4, at most about 5.5, at most about 5.6, at most about 5.7, at most about 5.8, at most about 5.9, at most about 6, at most about 6.1, at most about 6.2, at most about 6.3, at most about 6.4, at most about 6.5, at most about 6.6, at most about 6.7, at most about 6.8, at most about 6.9, at most about 7, at most about 7.1, at most about 7.2, at most about 7.3, at most about 7.4, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 9.2, at most about 9.4, at most about 9.6, at most about 9.8, at most about 10, at most about 10.2, at most about 10.4, at most about 10.6, at most about 10.8, at most about 11, at most about 11.2, at most about 11.4, at most about 11.6, at most about 11.8, or at most about 12.

In some cases, one or more detection probes may be detected. Detecting the one or more detection probes may comprise detecting or measuring a signal associated with the one or more detection probes. In some cases, one or more detection probe may interact with one or more other detection probes. The interaction between a detection probe and one or more detection probes may modify, increase, decrease, alter, or otherwise influence a signal associated with the detection probe. For example, a detection probe may comprise a detection moiety. The detection moiety of the detection probe may interact with another detection probe from another detection probe. The interaction between the detection moiety of the detection probe and the other detection moiety of the other detection probe may result in fluorescence resonance energy transfer (FRET). FRET may be used in any one of the methods described herein to detect one or more detection probes. A FRET signal may correspond to one or more subsequences corresponding to one or more analytes.

In some aspects, the methods described herein may comprise an amplifying step. The amplifying step may comprise amplifying the sequence comprising the one or more pluralities of subsequences to generate multiple copies of the sequence or a derivative thereof. The derivative thereof may comprise a complement or a reverse complement of the sequence. In cases where a complement or a reverse complement of the sequence is generated, detecting the sequence using the methods described herein may comprise detecting the complement or reverse complement of the sequence. The amplifying step may comprise performing a rolling circle amplification reaction. The rolling circle amplification reaction may be performed using a polymerase, e.g. a phi29 polymerase.

The rolling circle amplification reaction may be performed using one or more primers. The one or more primers may couple to or bind to one or more analytes (e.g. one or more RNA) at one or more positions. The one or more primers may couple to or bind to one or more binding moieties as described herein. The one or more primers may comprise a nucleic acid (e.g a single-stranded nucleic acid). The one or more primers may comprise DNA, RNA, or a combination thereof. The one or more primers may comprise one or more modifications. The one or more primers may have a length. The length of the one or more primers may comprise at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, or more. The one or more primers may comprise DNA, RNA, or a combination thereof. The one or more primers may comprise one or more modifications. The one or more primers may have a length. The length of the one or more primers may comprise at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, at most about 20 nucleotides, at most about 21 nucleotides, at most about 22 nucleotides, at most about 23 nucleotides, at most about 24 nucleotides, at most about 25 nucleotides, at most about 26 nucleotides, at most about 27 nucleotides, at most about 28 nucleotides, at most about 29 nucleotides, at most about 30 nucleotides, at most about 35 nucleotides, at most about 40 nucleotides, at most about 45 nucleotides, at most about 50 nucleotides, at most about 55 nucleotides, at most about 60 nucleotides, at most about 65 nucleotides, at most about 70 nucleotides, at most about 75 nucleotides, at most about 80 nucleotides, at most about 85 nucleotides, at most about 90 nucleotides, at most about 95 nucleotides, at most about 100 nucleotides, or more. The one or more primers may have a length of about 5 to about 100 nucleotides, about 6 to about 95 nucleotides, about 7 to about 90 nucleotides, about 8 to about 85 nucleotides, about 9 to about 80 nucleotides, about 10 to about 75 nucleotides, about 11 to about 70 nucleotides, about 12 to about 65 nucleotides, about 13 to about 60 nucleotides, about 14 to about 55 nucleotides, about 15 to about 50 nucleotides, about 16 to about 45 nucleotides, about 17 to about 40 nucleotides, about 18 to about 35 nucleotides, about 19 to about 30 nucleotides, about 20 to about 29 nucleotides, about 21 to about 28 nucleotides, about 22 to about 27 nucleotides, about 23 to about 26 nucleotides, or about 24 to about 25 nucleotides.

Another aspect of the present disclosure provides a method for identifying an analyte in a sample. The method may comprise contacting an analyte with a binding moiety. The binding moiety may comprise a probe that couples to the analyte. The probe may comprise a sequence. The sequence may comprise a plurality of subsequences. The method may comprise contacting the analyte with a primer. The primer may bind to or couple to the analyte. The primer may bind to or couple to the binding moiety. The binding moiety may comprise a first region that couples to or binds to the analyte. The binding moiety may comprise a second region that couples to the primer. The binding moiety may comprise a third region that couples to the primer. The primer may comprise a first region that couples to the analyte. The primer may comprise a second region that couples to or binds to the second region of the binding moiety. The primer may comprise a third region that couples to or binds to the third region of the binding moiety. The second region of the binding moiety may be ligated to the third region of the binding moiety. The second region of the binding moiety may be ligated to the third region of the binding moiety upon the second region of the binding moiety coupling to or binding to the second region of the primer and the third region of the binding moiety coupling to or binding to the third region of the primer. The ligation of the second region of the binding moiety to the third region of the binding moiety may generate a circular nucleic acid. The circular nucleic acid may be amplified by performing an amplification reaction. The amplification reaction may comprise a rolling circle amplification reaction. The rolling circle amplification reaction may comprise using the primer. The method may comprise binding the sample in contact with one or more detection probes (e.g. a first detection probe and a second detection probe). The first detection probe may couple to or bind to a first subsequence or derivative thereof (e.g. complement or reverse complement thereof) of the sequence. The first detection probe may comprise a first detection moiety. The second detection probe may couple to or bind to a second subsequence or derivative thereof (e.g. complement or reverse complement thereof) of the sequence. The second detection probe may comprise a second detection moiety. The first subsequence or derivative thereof may be adjacent to the second subsequence or derivative thereof. The method may comprise detecting the first detection moiety, the second detection moiety, or a combination thereof to identify at least the first subsequence or derivative thereof (e.g. complement or reverse complement thereof) and the second subsequence or derivative thereof (e.g. complement or reverse complement thereof). The method may comprise using the at least first subsequence or derivative thereof (e.g. complement or reverse complement thereof) and the second subsequence or derivative thereof (e.g. complement or reverse complement thereof) identified to identify the analyte.

The amplifying steps of the methods described herein may comprise binding a binding moiety to an analyte within a sample. The binding moiety may comprise a probe and a sequence. A binder may be added to the sample. The binder may comprise one or more pluralities of subsequences or complements or reverse complements thereof. The binder may couple to or bind to (e.g. hybridize to) the sequence of the binding moiety. The binder may comprise a nucleic acid, e.g. an oligonucleotide. The binder may comprise a padlock probe. A first end of the binder may be ligated to a second end of the binder to form a circular nucleic acid. For example, a ligase (e.g. a T4 ligase) may be added to the sample and used to ligate the first and second end of the padlock probe. The circular nucleic acid may be amplified to generate one or more amplicons. The one or more amplicons may comprise one or more subsequence or complements or reverse complements thereof. Detection probes may be added to the sample comprising the one or more amplicons. A first detection probe of the detection probes may couple or bind to a first subsequence or complement or reverse complement thereof. The first detection probe may comprise a first detection moiety. A second detection probe of the detection probes may couple or bind to a second subsequence or complement or reverse complement thereof. The second detection probe may comprise a second detection moiety. The first detection probe may be ligated to the second detection probe. The first detection moiety of the first detection probe and/or the second detection moiety of the second detection probe may be detected. The detection of the first detection moiety and/or the second detection moiety corresponding to the first subsequence or complement or reverse complement thereof and/or the second subsequence or complement or reverse complement thereof may be used to identify the analyte.

In some cases, the sample may be contacted with one or more detection probes. The contacting of the sample with one or more detection probes may result in the one or more detection probes coupling with one or more sequences of a binding moiety or a binder or amplification product of a binder (e.g. one or more amplicons). The binder or amplification product of a binder may comprise a sequence or complement or reverse complement thereof. In some cases, a first detection probe (e.g. the first detection probe) may bind to or couple to a sequence or complement or reverse complement thereof of a binding moiety or a sequence or complement or reverse complement thereof of a binder or an amplification product of a binder. A second detection probe (e.g. the second detection probe) may bind or couple to (1) the sequence or complement or reverse complement thereof of the binding moiety or (2) the sequence or complement or reverse complement thereof of the binder or the amplification product of the binder. The first detection probe may be bound to the sequence or complement or reverse complement thereof of the binding moiety or the sequence or complement or reverse complement thereof of the binder or the amplification product of the binder directly adjacent to where the second detection probe is bound (e.g. without any nucleobase spaces). In some cases, the first detection probe may be bound to the sequence or complement or reverse complement thereof of the binding moiety or the sequence or complement or reverse complement thereof of the binder or the amplification product of the binder with one or more nucleobases separated from where the second detection probe is bound. In cases where the first and second detection probes may be separated when bound to the sequence or complement or reverse complement thereof of the binding moiety or the sequence or complement or reverse complement thereof of the binder or the amplification product of the binder, a linker may be added to the sample. The linker may link the first and second detection probes together when the first and second detection probes are bound to the sequence or complement or reverse complement thereof of the binding moiety or the sequence or complement or reverse complement thereof of the binder or the amplification product of the binder. The linker may comprise a bifunctional linker comprising a first reactive chemical moiety on a first end of the linker and a second reactive chemical moiety on a second end of the linker. The first reactive chemical moiety of the linker may react with or interact with a first end of the first detection probe. The second reactive chemical moiety of the linker may react with or interact with a second end of the second detection probe. In some cases, the linker reacts with or interacts with both the first detection probe and the second detection probe to generate a linked detection probe complex. The linked detection probe complex may be detected during the detection steps of the methods described herein. In cases where the first and second detection probes may be separated when bound to the sequence or complement or reverse complement thereof of the binding moiety or the sequence or complement or reverse complement thereof of the binder or the amplification product of the binder, a gap-filling reaction may be performed to generate a linked nucleic acid sequence between the first detection probe and the second detection probe when bound. The gap-filling reaction may comprise adding a polymerase to the sample to gap-fill the nucleobases separated a first end of the first detection probe and a second end of the second detection probe. The product of the gap-filling reaction may comprise a contiguous nucleic acid sequence that comprises the first detection probe nucleic acid sequence and the second detection probe nucleic acid sequence. The gap-filled product may bind or couple stronger to the sequence or complement or reverse complement thereof of the binding moiety or the sequence or complement or reverse complement thereof of the binder or the amplification product of the binder as compared to either the first detection probe or second detection probe when not gap-filled.

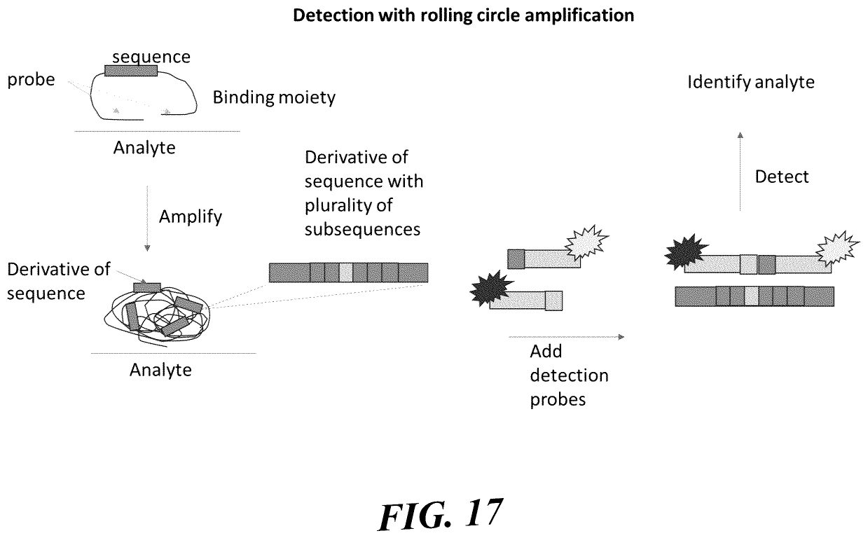

In some cases, rolling circle amplification may be used to identify an analyte. An example of methods that comprise using rolling circle amplification to identify the analyte is shown in FIG. 17. A binding moiety may be added to a sample. The sample may comprise the analyte. The binding moiety may comprise one or more probes and one or more sequences. The one or more probes may bind to or couple to (e.g. hybridize to) the analyte. In some cases, the binding moiety may comprise a padlock probe. The padlock probe may comprise a first end and a second end. The first end may couple or bind to a first portion of the analyte. The second end may couple or bind to a second portion of the analyte. In some cases, the first end may be directly adjacent to the second end when bound to the analyte (e.g. the first end may form a first double-stranded nucleic acid with the first portion of the analyte and the second end may form a second double-stranded nucleic acid with the second portion of the analyte and there is no single-stranded nucleotide between the first double stranded nucleic acid and the second double stranded nucleic acid.) In some cases, the first end may be separated by a gap from the second end when bound to the analyte. For example, the first end may form a first double-stranded nucleic acid with the first portion of the analyte and the second end may form a second double-stranded nucleic acid with the second portion of the analyte and there may be one or more single-stranded nucleotides between the first double stranded nucleic acid and the second double stranded nucleic acid (e.g. a gap). The first double-stranded nucleic acid and the second double-stranded nucleic acid may be extended to fill the gap. For example, a polymerase may be added to the sample. The polymerase may extend the first end of the padlock probe and/or the second end of the padlock probe to generate a gap-filled probe. The gap filled probe may be ligated (e.g. using a T4 ligase) to form a circular nucleic acid. In some cases, the padlock probe may be ligated to form a circular nucleic acid (e.g. without a gap-filling reaction.) The circular nucleic acid may be amplified to generate one or more amplicons. The one or more amplicons may comprise one or more subsequence or complements or reverse complements thereof. Detection probes may be added to the sample comprising the one or more amplicons. A first detection probe of the detection probes may couple or bind to a first subsequence or complement or reverse complement thereof. The first detection probe may comprise a first detection moiety. A second detection probe of the detection probes may couple or bind to a second subsequence or complement or reverse complement thereof. The second detection probe may comprise a second detection moiety. The first detection probe may be ligated to the second detection probe. The first detection moiety of the first detection probe and/or the second detection moiety of the second detection probe may be detected. The detection of the first detection moiety and/or the second detection moiety corresponding to the first subsequence or complement or reverse complement thereof and/or the second subsequence or complement or reverse complement thereof, respectively, may be used to identify the analyte.

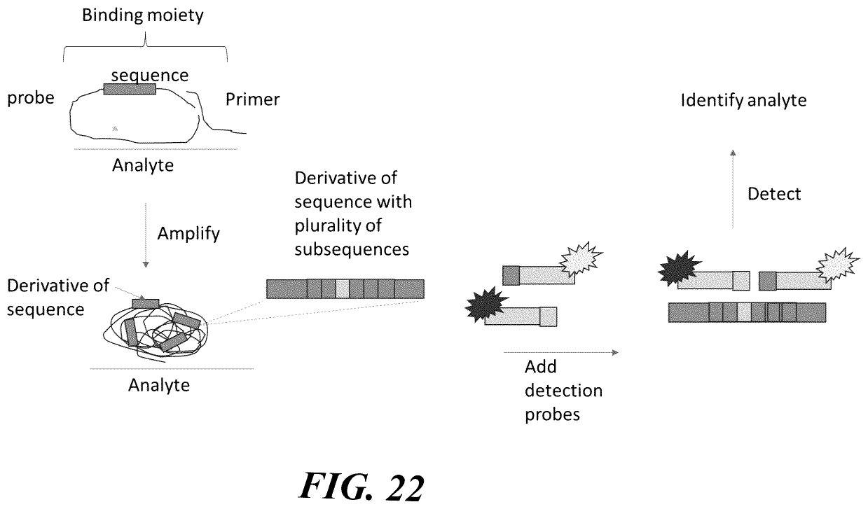

In some cases, rolling circle amplification may be used to identify an analyte. An example of methods that comprise using rolling circle amplification to identify the analyte is shown in FIG. 22. In some cases, a primer may be added to the sample. The primer may couple to or bind to the analyte. The primer may comprise nucleic acid (e.g. an oligonucleotide). A binding moiety may be added to a sample. The sample may comprise the analyte. The binding moiety may comprise one or more probes and one or more sequences. The one or more probes may bind to or couple to (e.g. hybridize to) the analyte. In some cases, the binding moiety may comprise a padlock probe. The padlock probe may comprise a first end and a second end. The first end may couple or bind to a first portion of the binding moiety. The second end may couple or bind to a second portion of the binding moiety. In some cases, the first end may be directly adjacent to the second end when bound to the binding moiety (e.g. the first end may form a first double-stranded nucleic acid with the first portion of the binding moiety and the second end may form a second double-stranded nucleic acid with the second portion of the binding moiety and there is no single-stranded nucleotide between the first double stranded nucleic acid and the second double stranded nucleic acid.) In some cases, the first end may be separated by a gap from the second end when bound to the binding moiety. For example, the first end may form a first double-stranded nucleic acid with the first portion of the binding moiety and the second end may form a second double-stranded nucleic acid with the second portion of the binding moiety and there may be one or more single-stranded nucleotides between the first double stranded nucleic acid and the second double stranded nucleic acid (e.g. a gap). The first double-stranded nucleic acid and the second double-stranded nucleic acid may be extended to fill the gap. For example, a polymerase may be added to the sample. The polymerase may extend the first end of the padlock probe and/or the second end of the padlock probe to generate a gap-filled probe. The gap filled probe may be ligated (e.g. using a T4 ligase) to form a circular nucleic acid. In some cases, the padlock probe may be ligated to form a circular nucleic acid (e.g. without a gap-filling reaction.) The circular nucleic acid may be amplified to generate one or more amplicons. The primer may be used to amplify the circular nucleic acid to generate the one or more amplicons. The one or more amplicons may comprise one or more subsequence or complements or reverse complements thereof. Detection probes may be added to the sample comprising the one or more amplicons. A first detection probe of the detection probes may couple or bind to a first subsequence or complement or reverse complement thereof. The first detection probe may comprise a first detection moiety. A second detection probe of the detection probes may couple or bind to a second subsequence or complement or reverse complement thereof. The second detection probe may comprise a second detection moiety. The first detection probe may be ligated to the second detection probe. The first detection moiety of the first detection probe and/or the second detection moiety of the second detection probe may be detected. The detection of the first detection moiety and/or the second detection moiety corresponding to the first subsequence or complement or reverse complement thereof and/or the second subsequence or complement or reverse complement thereof may be used to identify the analyte.

In some aspects, a detection moiety of a detection probe (e.g. the first detection moiety of the first detection probe) may be detected. A variety of methods may be used to detect the detection moiety of the detection probe (e.g. the first detection moiety of the first detection probe) including but not limited to imaging the sample, scanning the sample, sequencing the sample, or a combination thereof. In some cases, detecting the detection moiety may comprise imaging using one or more imaging systems, including but not limited to a microscope, a scanner, or fluorimeter. The microscope may be a fluorescence microscope, a confocal microscope, an upright microscope, an inverted microscope, or a combination thereof. The imaging system may further comprise a fluidic system, wherein the fluidic system may comprise automated processing of fluid handling associated with the methods described herein. In some cases, the imaging may result in imaging data. The imaging data may comprise an image, a table, a graph, a quantitative metric, a database, or a combination thereof. The imaging data may comprise quantitative information. The imaging data may comprise spatial location information.

In some cases, the methods described herein may comprise detecting a change in signal of one or more detection moieties. In some aspects, detecting a first detection moiety and/or a second detection moiety (e.g. the first detection moiety and/or the second detection moiety) of a first detection probe and/or a second detection probe (e.g. a first detection probe and/or a second detection probe) may comprise detecting a ratio of a signal associated with the first detection moiety and/or the second detection moiety. For example, a signal associated with the first detection moiety may be divided by a signal associated with the second detection moiety to detect a ratio of the first detection moiety and the second detection moiety. In some cases, a count of the first detection moiety may be divided by a count of the second detection moiety to determine a ratio of the first detection moiety and the second detection moiety. The ratio of the first detection moiety and the second detection moiety may be used to identify the analyte. As another example, a signal associated with the second detection moiety may be divided by a signal associated with the first detection moiety to detect a ratio of the second detection moiety and the first detection moiety. In some cases, a count of the second detection moiety may be divided by a count of the first detection moiety to determine a ratio of the second detection moiety and the first detection moiety. The ratio of the second detection moiety and the first detection moiety may be used to identify the analyte. As another example, a signal associated with the third detection moiety may be divided by a signal associated with the fourth detection moiety to detect a ratio of the third detection moiety and the fourth detection moiety. In some cases, a count of the third detection moiety may be divided by a count of the fourth detection moiety to determine a ratio of the third detection moiety and the fourth detection moiety. The ratio of the third detection moiety and the fourth detection moiety may be used to identify the analyte. As another example, a signal associated with the fourth detection moiety may be divided by a signal associated with the third detection moiety to detect a ratio of the fourth detection moiety and the third detection moiety. In some cases, a count of the fourth detection moiety may be divided by a count of the third detection moiety to determine a ratio of the fourth detection moiety and the third detection moiety. The ratio of the fourth detection moiety and the third detection moiety may be used to identify the analyte.

In some cases, the signal associated with the first detection moiety may be a measurement of the concentration of the first detection moiety, percentage of a maximum of the first detection moiety, or a combination thereof. The signal associated with a detection moiety (e.g. the first detection moiety and/or second detection moiety) may be used to determine the presence and/or quantity of a subsequence (e.g. the first subsequence and/or second subsequence). In some cases, detecting a ratio of one detection moiety (e.g. a first detection moiety) to another detection moiety (e.g. a second detection moiety) may comprise detecting a ratio of the one detection moiety to another detection moiety at a given spatial location. For example, a signal of a first detection probe may be measured at a particular pixel location in an image and a signal of a second detection probe may be measured at a particular pixel location in the image. The signal of the first detection probe at the particular pixel location may be divided by or otherwise compared to the signal of the second detection probe at the particular pixel location to generate to generate a ratio or correspondence of the first detection probe to the second detection probe at the particular location. The ratio or correspondence of the first detection probe to the second detection probe at the particular location may be used to identify the analyte.

In some embodiments, the given spatial location may comprise a pixel, a set of pixels, a region of interest, or a combination thereof. In some cases, a first ratio of the first detection moiety and/or the second detection moiety may denote an identity of a first combination of the first subsequence and/or the second subsequence. In some cases, a second ratio of the first detection moiety and/or the second detection moiety may denote an identity of a second combination of the first subsequence and/or the second subsequence. The first combination of the first subsequence and/or the second subsequence and the second combination of the first subsequence and/or second subsequence may be used to determine the identity and/or presence and/or absence of the one or more analytes. For example, the detection of one or more subsequences may enable the identification of the one or more subsequences. The identity of the one or more subsequences may be compared against a codebook and/or reference that contains combinations of subsequences correlated to one or more analytes. The comparison of the one or more subsequences to the codebook and/or reference may enable identification of one or more analytes.

In some aspects detecting a first detection moiety and/or a second detection moiety (e.g. the first detection moiety and/or the second detection moiety) of a first detection probe and/or a second detection probe (e.g. a first detection probe and/or a second detection probe) may comprise counting, quantifying, estimating, and/or determining the number of nucleotides detected. In some cases, detecting a first detection moiety and/or a second detection moiety (e.g. the first detection moiety and/or the second detection moiety) of a first detection probe and/or a second detection probe (e.g. a first detection probe and/or a second detection probe), respectively, may comprise counting the number of each type of nucleotide detected. Counting the number of each type of nucleotide detected may involve counting the number of adenines (A), cytosines (C), guanines (G), thymines (T), or a combination thereof associated with a sequence or complement or reverse complement thereof. In some aspects, detecting a first detection moiety and/or a second detection moiety (e.g. the first detection moiety and/or the second detection moiety) of a first detection probe and/or a second detection probe (e.g. a first detection probe and/or a second detection probe) may comprise counting, quantifying, estimating, and/or determining the number of dinucleotides detected associated with a sequence or complement or reverse complement thereof. In some cases, detecting a first detection moiety and/or a second detection moiety (e.g. the first detection moiety and/or the second detection moiety) of a first detection probe and/or a second detection probe (e.g. a first detection probe and/or a second detection probe) may comprise counting, quantifying, estimating, and/or determining the number of each type of dinucleotide detected. Counting, quantifying, estimating, and/or determining the number of each type of nucleotide detected may involve counting the number of AA's, AC's, AG's, AT's, CC's, CA's, CT's, CG's, GG's, GA's, GT's, GC's, TT's, TA's, TC's, TG's, or a combination thereof associated with a sequence or complement or reverse complement thereof. In some cases, combination of three nucleotides may be counted, quantified, estimated and/or determined. For example, the following illustrative trinucleotide combinations may be counted, quantified, estimated, or otherwise determined: AAA, AAT, AAC, AAG, ATA, ACA, AGA, TAA, CAA, GAA, ATT, ACC, AGG, ATC, ATG, GGC, GCA, GTC, CTG, GTA, GAA, and CCC. In some cases, combination of four nucleotides may be counted, quantified, estimated and/or determined (e.g. ACGT may be a combination) associated with a sequence or complement or reverse complement thereof. In some cases, combination of five nucleotides may be counted, quantified, estimated and/or determined (e.g. CGTCA may be a combination) associated with a sequence or complement or reverse complement thereof. In some cases, a nucleotide, dinucleotide, trinucleotide, combination of four nucleotides, and/or combination of five nucleotides may be associated with a specific fluorescent dye or combination of fluorescent dyes. For example, the dinucleotide TT may be associated with a Cy3 detection moiety. In some cases, a nucleotide, dinucleotide, trinucleotide, combination of four nucleotides, and/or combination of five nucleotides may be associated with a specific fluorescent dye or combination of fluorescent dyes and/or the corresponding stoichiometry of the specific fluorescent dye or combination of fluorescent dyes. In some cases, the nucleotides or dinucleotides are counted across multiple rounds of detection. The detecting a first detection moiety and/or a second detection moiety (e.g. the first detection moiety and/or the second detection moiety) of a first detection probe and/or a second detection probe (e.g. a first detection probe and/or a second detection probe) may be performed without information related to the temporal order of detection round. For example, detecting the first detection moiety and the second detection moiety may comprise counting the instances of detection without tracking the order of detection. The analyte may be identified based on detecting as described herein without information related to the temporal order of detection rounds. The detecting may comprise performing a computer processing to analyze data generated from the one or more detection probes.

In some aspects, the methods described herein may comprise removing the detection probes from binding moiety after detecting. The removing may comprise incubation with a chemical and/or solvent, incubation at a temperature, addition of a removal probe, addition or an enzyme, or a combination thereof. The chemical and/or solvent may comprise dimethyl sulfoxide, guanidine hydrochloride, urea, formamide, NaCl, MgCl₂, NaOH, or a combination thereof. The temperature during probe removal may be at least about 20° C., at least about 25° C., at least about 30° C., at least about 35° C., at least about 40° C., at least about 45° C., at least about 50° C., at least about 55° C., at least about 60° C., at least about 65° C., at least about 70° C., at least about 75° C., at least about 80° C., at least about 85° C., at least about 90° C., or at least about 95° C. The removal probe may comprise a nucleic acid sequence that is at least partially complementary to the sequence of the binding moiety. The temperature during probe removal may be at most about 20° C., at most about 25° C., at most about 30° C., at most about 35° C., at most about 40° C., at most about 45° C., at most about 50° C., at most about 55° C., at most about 60° C., at most about 65° C., at most about 70° C., at most about 75° C., at most about 80° C., at most about 85° C., at most about 90° C., or at most about 95° C. The removal probe may comprise a nucleic acid sequence that is at most partially complementary to the sequence of the binding moiety. The removal probe may comprise a toehold, wherein the toehold is complementary to at least a portion of the binding moiety. The enzyme may comprise a DNAse, an RNAse, an exonuclease, an endonuclease, or a combination thereof.

In some aspects, the methods described herein may comprise contacting the sample with one or more pluralities of binding moieties. In some cases, a first binding moiety of the one or more pluralities of binding moieties may recognize a different analyte compared to a second binding moiety of the one or more pluralities of binding moieties (e.g. the first binding moiety may recognize one RNA and the second binding moiety may recognize a second RNA or the first binding moiety may recognize a protein and the second binding moiety may recognize an RNA). In some cases, each biding moiety of the one or more pluralities of binding moieties may comprise a different sequence. In some embodiments, the one or more pluralities of binding moieties may comprise at least two binding moieties that recognize the same analyte. In some embodiments, the one or more pluralities of binding moieties may comprise at least two binding moieties that recognize a different analyte (e.g. a first binding moiety recognizes a first analyte and a second binding moiety recognizes a second analyte and the first analyte and the second analyte are different compositions). In some embodiments, the one or more pluralities of binding moieties may comprise at least two binding moieties that comprise the same sequence. In some embodiments, the one or more pluralities of binding moieties may comprise at least two binding moieties that comprise a different sequence (e.g. not the same sequence).

In some aspects, additional subsequences of the one or more pluralities of subsequences may be detected using the methods described herein. In some cases, additional subsequences may be detected across additional rounds (e.g. cycles) of detection. For example, a second round of detection may be performed to detect a third subsequence and/or a fourth subsequence using a third detection probe and/or a fourth detection probe to identify the third subsequence and/or fourth subsequence, respectively. The third subsequence and/or fourth subsequence may comprise a nucleic acid and/or have similar characteristics as those described herein in relation to the first subsequence and/or second subsequence. The third detection probe may comprise a third detection moiety. The fourth detection probe may comprise a fourth detection moiety. In some embodiments, the third detection moiety and/or fourth detection moiety may have similar characteristics as the first detection moiety and/or second detection moiety as described herein. The third subsequence may be adjacent to the first and/or second subsequence as described herein. The fourth subsequence may be adjacent to the first and/or second subsequence as described herein. In some embodiments, the first, second, third and/or fourth subsequences each comprise at least one nucleotide associated with a given nucleic acid. In some cases, the first, second, third, and/or fourth subsequences are directly adjacent to each other in a nucleic acid molecule.

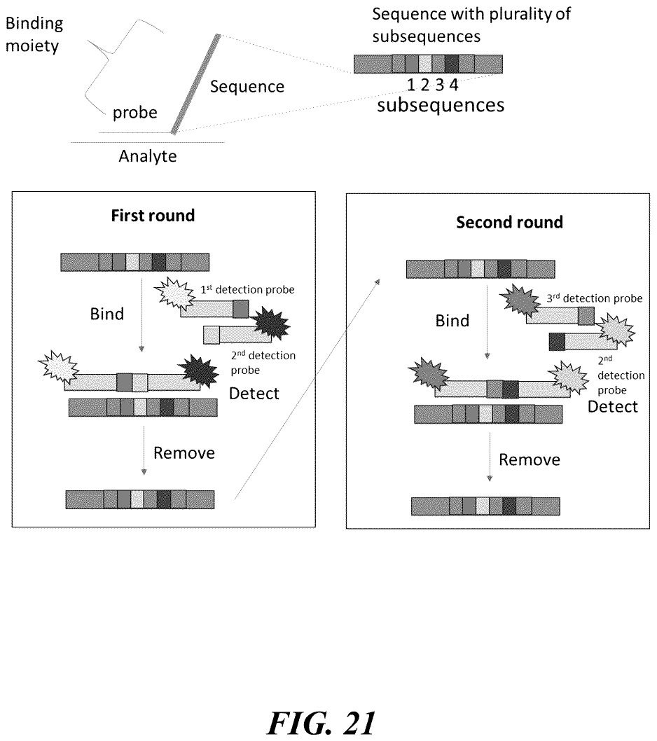

An example of performing more than one round of detection is shown in FIG. 21. In this case, a binding moiety binds or couples to an analyte. The binding moiety may comprise a probe and/or a sequence. The probe may bind the analyte. The sequence may comprise one or more pluralities of subsequences. For example, the sequence may comprise four subsequences, as shown in FIG. 21. A first detection probe and a second detection probe may be added to the sample with the analyte. The first detection probe may bind to the first subsequence. The second detection probe may bind to the second subsequence. The first detection probe may comprise a first detection moiety. The second detection probe may comprise a second detection moiety. FIG. 21 shows an example where the first detection moiety is different than the second detection moiety (e.g. the first detection moiety absorbs light at a different wavelength from the second detection moiety). In some cases, the first detection moiety may be the same as the second detection moiety (e.g. the first detection moiety comprises the same composition as the second detection moiety). The first and the second detection moieties may be detected. The first detection probe and the second detection probe may be removed. A second round of detection may be performed after the first round of detection. A third detection probe and a fourth detection probe may be added to the sample with the analyte. The third detection probe may bind to the third subsequence. The fourth detection probe may bind to the fourth subsequence. The third detection probe may comprise a third detection moiety. The fourth detection probe may comprise a fourth detection moiety. FIG. 21 shows an example where the third detection moiety is different than the fourth detection moiety (e.g. the third detection moiety absorbs light at a different wavelength from the fourth detection moiety). In some cases, the third detection moiety may be the same as the fourth detection moiety (e.g. the third detection moiety comprises the same composition as the fourth detection moiety). The third and the fourth detection moieties may be detected. The third detection probe and the fourth detection probe may be removed. The information from the first round and/or the second round of detection may be used to identify the analyte.

In some aspects, the first and/or the second detection moieties as described herein may be the same (e.g. comprise the same composition, comprise the same molecular weight, or comprise the same chemical formula). In some aspects, the first and/or the second detection moieties as described herein may be different (e.g. comprise different chemical compositions, or comprise different molecular weights). In some cases, wherein the first and/or second detection moieties are different, the first and/or second detection moieties may comprise features that are the same and/or different. In some cases, the first detection moiety comprises first a linker. The first linker may link the detection moiety of the first detection moiety to a nucleic acid of the first detection moiety. In some cases, the first linker may comprise one or more phosphodiester linkages, one or more ester linkages, one or more amide linkages, one or more ether linkages, one or more PEG linkages, one or more methylene linkages, or a combination thereof. In some cases, the second detection moiety comprises second a linker. The second linker may link the detection moiety of the second detection moiety to a nucleic acid of the second detection moiety. In some cases, the second linker may comprise one or more phosphodiester linkages, one or more ester linkages, one or more amide linkages, one or more ether linkages, one or more PEG linkages, one or more methylene linkages, or a combination thereof. In some cases, the first linker and the second linker may be the same (e.g. the first linker may comprise the same chemical composition as the second linker, the first linker may comprise the same linkages as the second linker, the first linker may comprise the same chemical formula as the second linker, the first linker may comprise the same molecular weight as the second linker, or a combination thereof). In some cases, the first linker and the second linker may be the different (e.g. the first linker may not comprise the same chemical composition as the second linker, the first linker may not comprise the same linkages as the second linker, the first linker may not comprise the same chemical formula as the second linker, the first linker may not comprise the same molecular weight as the second linker, or a combination thereof.)

In some cases, the first detection moiety may comprise a first fluorescent dye and the second detection moiety comprise a second fluorescent dye. In some cases, the first fluorescent dye may be the same as the second fluorescent dye. For example, the first fluorescent dye may comprise the same chemical composition as the second fluorescent dye. The first fluorescent dye may comprise the same chemical formula as the second fluorescent dye. The first fluorescent dye may comprise the same molecular weight as the second fluorescent dye. The first fluorescent dye may emit at the same wavelength as the second fluorescent dye. The first fluorescent dye may absorb at the same wavelength as the second fluorescent dye. In some cases, the first fluorescent dye may be the same as the second fluorescent dye for any one or a combination thereof of the preceding reasons.

In some cases, the first fluorescent dye may be different from the second fluorescent dye. For example, the first fluorescent dye may not comprise the same chemical composition as the second fluorescent dye. The first fluorescent dye may not comprise the same chemical formular as the second fluorescent dye. The first fluorescent dye may not comprise the same molecular weight as the second fluorescent dye. The first fluorescent dye may not emit at the same wavelength as the second fluorescent dye. The first fluorescent dye may not absorb at the same wavelength as the second fluorescent dye. In some cases, the first fluorescent dye may be different from the fluorescent dye for any one or a combination thereof of the preceding reasons.

In some aspects, the third and/or the fourth detection moieties as described herein may be the same (e.g. comprise the same composition, comprise the same molecular weight, or comprise the same chemical formula). In some aspects, the third and/or the fourth detection moieties as described herein may be different (e.g. comprise different chemical compositions, or comprise different molecular weights). In some cases, wherein the third and/or fourth detection moieties are different, the third and/or fourth detection moieties may comprise features that are the same and/or different. In some cases, the third detection moiety comprises third a linker. The third linker may link the detection moiety of the third detection moiety to a nucleic acid of the third detection moiety. In some cases, the third linker may comprise one or more phosphodiester linkages, one or more ester linkages, one or more amide linkages, one or more ether linkages, one or more PEG linkages, one or more methylene linkages, or a combination thereof. In some cases, the fourth detection moiety comprises fourth a linker. The fourth linker may link the detection moiety of the fourth detection moiety to a nucleic acid of the fourth detection moiety. In some cases, the fourth linker may comprise one or more phosphodiester linkages, one or more ester linkages, one or more amide linkages, one or more ether linkages, one or more PEG linkages, one or more methylene linkages, or a combination thereof. In some cases, the third linker and the fourth linker may be the same (e.g. the third linker may comprise the same chemical composition as the fourth linker, the third linker may comprise the same linkages as the fourth linker, the third linker may comprise the same chemical formula as the fourth linker, the third linker may comprise the same molecular weight as the fourth linker, or a combination thereof). In some cases, the third linker and the fourth linker may be the different (e.g. the third linker may not comprise the same chemical composition as the fourth linker, the third linker may not comprise the same linkages as the fourth linker, the third linker may not comprise the same chemical formula as the fourth linker, the third linker may not comprise the same molecular weight as the fourth linker, or a combination thereof.)

In some cases, the third detection moiety may comprise a third fluorescent dye and the fourth detection moiety comprise a fourth fluorescent dye. In some cases, the third fluorescent dye may be the same as the fourth fluorescent dye. For example, the third fluorescent dye may comprise the same chemical composition as the fourth fluorescent dye. In some cases, the third fluorescent dye may comprise the same chemical formula as the fourth fluorescent dye. The third fluorescent dye may comprise the same molecular weight as the fourth fluorescent dye. In some cases, the third fluorescent dye may emit at the same wavelength as the fourth fluorescent dye. The third fluorescent dye may absorb at the same wavelength as the fourth fluorescent dye. In some cases, the third fluorescent dye may be the same as the fourth fluorescent dye for any one or a combination thereof of the preceding reasons.

In some cases, the third fluorescent dye may be different from the fourth fluorescent dye. For example, the third fluorescent dye may not comprise the same chemical composition as the fourth fluorescent dye. The third fluorescent dye may not comprise the same chemical formular as the fourth fluorescent dye. The third fluorescent dye may not comprise the same molecular weight as the fourth fluorescent dye. The third fluorescent dye may not emit at the same wavelength as the fourth fluorescent dye. The third fluorescent dye may not absorb at the same wavelength as the fourth fluorescent dye. In some cases, the third fluorescent dye may be different from the fourth fluorescent dye for any one or a combination thereof of the preceding reasons.

In another aspect, the present disclosure provides a reagent composition for identifying one or more analytes in a sample. The reagent composition may comprise one or more binding moieties as disclosed herein. A binding moiety of the one or more binding moieties may comprise one or more probes, one or more sequences, or a combination thereof, as disclosed herein. The one or more probes may comprise nucleic acid, one or more polypeptide, or a combination thereof, as disclosed herein. The one or more sequences of the binding moiety may comprise nucleic acid, as disclosed herein. The reagent composition may comprise one or more detection probes, as disclosed herein. The reagent composition may comprise a first detection probe having a first detection moiety, a second detection probe having a second detection moiety, a third detection probe having a third detection moiety, a fourth detection probe comprising a fourth detection moiety, or any combination thereof, as disclosed herein. The reagent composition may comprise one or more binders. The reagent composition may comprise one or more ligases, as disclosed herein. The reagent composition may comprise one or more polymerases, as disclosed herein. The reagent composition may comprise one or more buffers, as disclosed herein. The reagent composition may comprise one or more chaotropic agents, as disclosed herein. The reagent composition may comprise one or more samples, as disclosed herein. The reagent composition may comprise one or more polymers, as disclosed herein. The reagent composition may comprise one or more solvents, as disclosed herein. The reagent composition may comprise one or more RNAse, as disclosed herein. The reagent composition may comprise one or more DNAse, as disclosed herein.

In another aspect, the present disclosure provides a reaction mixture comprising a reagent composition disclosed herein and further comprising one or more analytes. The one or more analytes of the reaction mixture may be contained within the sample. For example, the one or more analytes may be within, on the surface of, associated with, or covalently attached to a sample. The sample may be a tissue sample, as described herein. The sample may be a tissue sample embedded in a hydrogel, as described herein.

In another aspect, the present disclosure provides a kit for identifying one or more analytes. The kit may comprise one or more reagent compositions disclosed herein. The kit may comprise one or more samples as described herein. The kit may comprise instructions for directing the use of the reagent composition for identifying one or more analytes as described herein. The instructions may comprise directions for performing one or more operations associated with the methods described herein. The kit may comprise one or more sample holders. The kit may comprise one or more well plates for mounting one or more samples as described herein. The kit may comprise one or more flow cells.

### Computer Systems

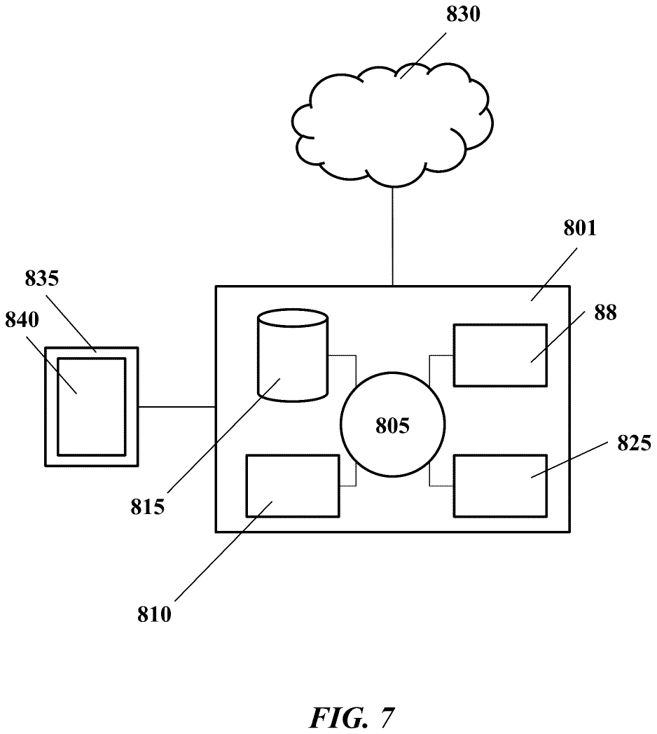

The present disclosure provides computer systems that are programmed to implement methods of the disclosure. FIG. 7 shows a computer system 801 that is programmed or otherwise configured to control protein expression in plant seeds. The computer system 801 can regulate various aspects of plant seed growth and protein extraction of the present disclosure, such as, for example, temperature control, humidity control, pumping speed, column pressure, exposure to light, access to water, or a combination thereof. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 88 (e.g., network adapter) for communicating with one or more other systems, and peripheral devices 825, such as cache, other memory, data storage and/or electronic display adapters. The memory 810, storage unit 815, interface 88 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 88. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user (e.g., an automated greenhouse). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine-readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, operating parameters and conditions, options to control the temperature, humidity level or flow rate, volume dispensed, process progress, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, calculate values, measure variances, analyze image data, analyze tabulated data, measure minimum values, measure maximum values, analyze mass spectrometry data, or calculate flow rates.

### EXAMPLES

### Example 1: Sequence Identification without Subsequence Order Information Across Four States Per Detection Round

In this Example, 165 target sequences will be identified using 4-state decoding and 8 rounds of detection without the need of arranging the detection rounds in specific temporal order.

A sample comprising at least 165 target sequences will be incubated with a plurality of binding moieties which each comprise a barcode sequence. The barcode sequences will include the following base sequence: 3′-CATGCTACGN₁N₂N₃N₄GCATACGCTTGATCGN₅N₆N₇N₈GATCAATACGG-5′, where each G, T, A, and C is representative of guanine (G), thymine (T), adenine (A), and cytosine (C), respectively. Each Nᵢ is representative of any one of A, C, T, or G at the given position. The identity of each target sequence will be represented by a particular quantity of each nucleotide detected in any particular order. For example, a first target sequence could be represented by 2 A's, 3 T's, 1 C, and 2G's, a second target sequence could be represented by 3 A's, 1 T, 2 C's, and 2 G's, a third target sequence could be represented by 0 A's, 1 T, 4 C's, and 3 G's, and a fourth target sequence could be represented by 3 A's, 1 T, 1 C, and 3G's. This identification scheme enables detection of 165 unique target sequences using 4-states. The number of rounds performed as a function of the plex level of a given target sequence set is shown in FIG. 1 for different numbers of states.

The binding moieties will each recognize a unique target sequence. Each corresponding barcode sequence will be uniquely correlated to the target sequence by the quantity of nucleotides within each variable region of the barcode sequence. Detection of the barcode sequence will include 8 rounds of detection. Each round of detection will comprise incubation of a population of decoding probes and a population of anchoring probes. The population of decoding probes will be the same for every round of detection and will include the following probe sequences: 5′Dye1-NNNNNNNNA 3′, 5′Dye2-NNNNNNNNT 3′, 5′Dye3-NNNNNNNNC 3, and 5′Dye4-NNNNNNNNG 3′, wherein Dye1, Dye2, Dye3, and Dye4, are spectrally distinct and wherein N is represented by any one of A, C, T, or G. Each round of detection will include the addition of a given anchoring probe population, where, the population in the first round will be 5′-p-NNNCGTATGC/invT/, the population in the second round will be 5′-p-NNCGTATGCG/invT/, the population in the third round will be 5′-p-NCGTATGCGA/invT/, the population in the fourth round will be 5′-p-CGTATGCGAA/invT/, the population in the fifth round will be 5′-p-NNNCTAGTTA/invT/, the population in the sixth round will be 5′-p-NNCTAGTTAT/invT/, the population in the seventh round will be 5′-p-NCTAGTTATG/invT/, and the population in the eighth round will be 5′-p-CTAGTTATGC/invT/. For each of the anchoring probes, 5′ end includes a phosphorylation modification (given by -p-) and 3′ end includes an inverted thymine (given by/invT/). The 5′ phosphorylation modification enables ligation of the anchoring probe to the decoding probes. The 3′ inverted thymine prevents extension and/or ligation.

The anchor and decoding probes will couple or bind to the barcode sequences within the sample corresponding to each of the target sequences. After binding, the bound probes will be ligated, and the non-ligated probes will be washed. The sample will be imaged using a fluorescence microscope. The signal detected at each location will be associated with the given terminal nucleotide of the decoding probe corresponding to each position of the barcode. After completing eight rounds of detection, the signals at each location corresponding to each of Dyes 1-4 across all rounds will be analyzed. The analysis will involve correlating the dye signal at each location to a nucleotide identity and counting the frequency of each nucleotide at each location. The total count of each nucleotide at each location will be compared to the total nucleotide count within each barcode sequence corresponding to each of the target sequences. Each location within the sample with a detectable signal will be identified as one of the target sequences based on the number of each nucleotide detected.

### Example 2: Sequence Identification without Subsequence Order Information Across Ten States Per Detection Round

In this example, 715 target sequences will be identified using a 10-state decoding and 4 rounds of detection without the need of arranging the detection rounds in specific temporal order.

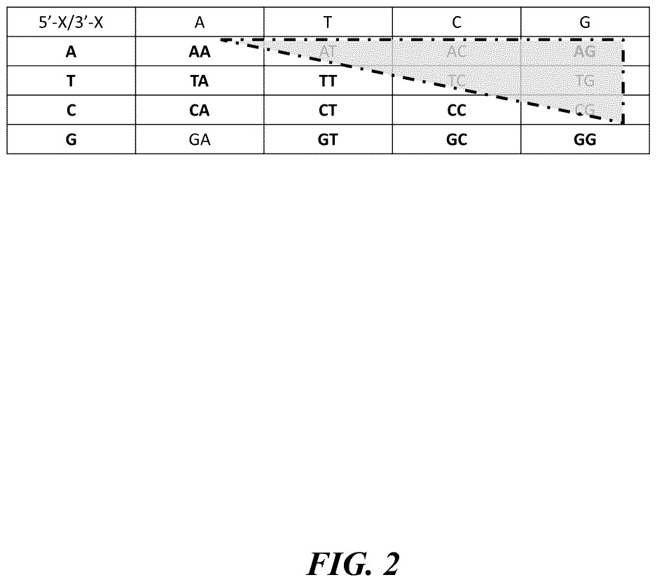

A sample comprising at least 715 target sequences will be incubated with a plurality of binding moieties which each comprise a barcode sequence. The barcode sequences will include the following base sequence: 3′ CATGCTACG(N₁N₂)(N₃N₄)GCATACGCTTGATCG(N₅N₆)(N₇N₈)GATCAATACGG-5′, where each G, T, A, and C is representative of guanine (G), thymine (T), adenine (A), and cytosine (C), respectively. Each Nᵢ is representative of any one of A, C, T, or G at the given position. The identity of each target sequence will be represented by a particular quantity of each of 10 dinucleotides detected in any particular order. The detected dinucleotides are combinations of the barcode sequence that are given by N₁N₂, N₃N₄, N₅N₆, and N₇N₈. For example, a first target sequence could be represented by 1 AT, 1 TC, 1 CG, and 1 AA, a second target sequence could be represented by 1 AT, 1 TT, 1 CA, and 1 AG, a third target sequence could be represented by 1 AC, 1 AC, 1 CG, and 1 GG and a fourth target sequence could be represented by 1 AC, 1 AT, 1 TC, and 1 AG. This identification scheme enables detection of 715 target sequences across 4 cycles. The possible dinucleotide combinations are given in FIG. 2. In this detection scheme, certain dinucleotide combinations may not be distinguishable from others, including TA vs. AT, e.g. Thus, the 10-state detection scheme depicted in this example eliminates 6 of the possible 16 dinucleotide combinations from the barcode possibilities.

The binding moieties will each recognize a unique target sequence. Each corresponding barcode sequence will be uniquely correlated to the target sequence by the quantity of dinucleotides within each variable region of the barcode sequence. Detection of the barcode sequence will include 4 rounds of detection. Each round of detection will comprise incubation of two populations of decoding probes. In the first round of detection, the first population of decoding probes will include 5′-Dye1-GTACGATGCA-3′, 5′-Dye2-GTACGATGCT-3′, 5′-Dye3-GTACGATGCC-3′, and 5′-Dye4-GTACGATGCG-3, and the second population of decoding probes will include 5′-p-ANNCGTATGC-3′Dye1, 5′-p-TNNCGTATGC-3′Dye2, 5′-p-CNNCGTATGC-3′Dye3, and 5′-p-GNNCGTATGC-3′Dye4. Each of Dye1, Dye2, Dye3, and Dye4 will be a fluorescent dye and be spectrally distinct. Each of the second population of decoding probes will include a 5′ phosphorylation group (given by -p-). In each nucleic acid sequence, N can be any one of the four nucleotides (A, C, T, or G) within different molecules of the population of the probe. In the second round of detection, the first population of decoding probes will include 5′-Dye1-ACGATGCNNA-3′, 5′-Dye2-ACGATGCNNT-3′, 5′-Dye3-ACGATGCNNC-3′, and 5′-Dye4-ACGATGCNNG-3′, and the second population of decoding probes will include 5′-p-ACGTATGCGA-3′Dye1, 5′-p-TCGTATGCGA-3′Dye2, 5′-p-CCGTATGCGA-3′Dye3, and 5′-p-GCGTATGCGA-3′Dye4. Each additional round of detection will use similar populations of decoding probes. For each round of detection, probes from each of populations 1 and 2 will couple or bind to the barcode sequences within the sample corresponding to each of the target sequences. After binding, the bound probes will be ligated, and the non-ligated probes will be washed. The sample will be imaged using a fluorescence microscope. The ligated decoding probes from each round will be removed prior to addition of the decoding probes for the next cycle.

The signal(s) detected at each location during each cycle will be associated with a given dinucleotide sequence. Each nucleotide is uniquely associated with one of Dyes1-4, and the combination of dye signals detected corresponds to one of the 10 possible dinucleotides. After completing four rounds of detection, the signals at each location corresponding to each of Dyes 1-4 across all rounds will be analyzed. The analysis will involve correlating the combination of dye signals at each location during each round to a dinucleotide identity and counting the frequency of each dinucleotide at each location. The total count of each dinucleotide at each location will be compared to the total dinucleotide count within each barcode sequence corresponding to each of the target sequences. Each location within the sample with a detectable signal will be identified as one of the target sequences based on the number of each dinucleotide detected.

### Example 3: Sequence Identification without Subsequence Order Information Across Sixteen States per Detection Round

In this example, 3876 target sequences will be identified using a 16-state decoding and 4 rounds of detection without the need of arranging the detection rounds in specific temporal order.

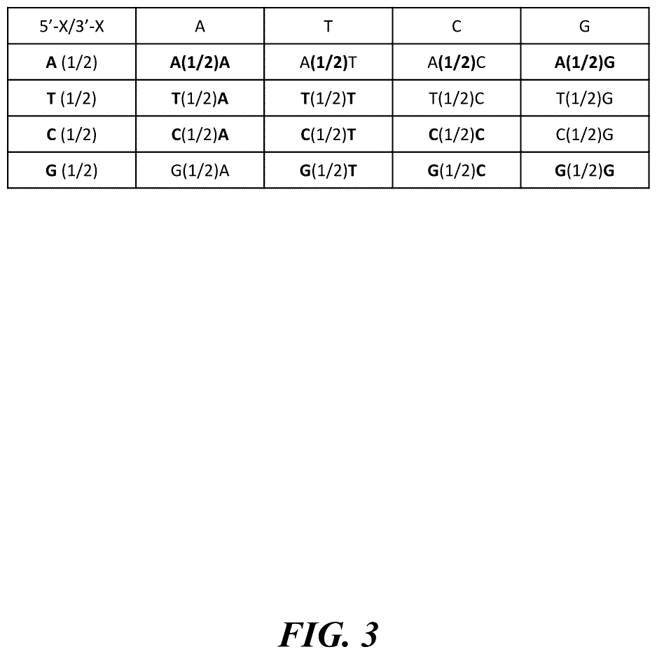

A sample comprising at least 3876 target sequences will be incubated with a plurality of binding moieties which each comprise a barcode sequence. The barcode sequences will include the following base sequence: 3′ CATGCTACG(N₁N₂)(N₃N₄)GCATACGCTTGATCG(N₅N₆)(N₇N₈)GATCAATACGG-5′, where each G, T, A, and C is representative of guanine (G), thymine (T), adenine (A), and cytosine (C), respectively. Each Nᵢ is representative of any one of A, C, T, or G at the given position. The identify of each target sequence will be represented by a quantity of each of 16 dinucleotides detected in any particular order. Each nucleotide of each dinucleotide will have a specific dye associated with it. The dye in each case may differ by either the dye molecule or the number of dye molecules associated with the detection event of the corresponding nucleotide. In each case, the dinucleotide will be detected by ligating two decoding probes together which hybridize to regions of the barcode sequence that overlap with the variable region corresponding to the dinucleotide to be detected. The 3′ terminal nucleotide of one decoding probe and 5′ terminal nucleotide of the other decoding probe will represent the sequence of the particular dinucleotide. The type and number of dinucleotides associated with each nucleotide of the dinucleotide combination will relate to the states of the 16-state decoding scheme. An example of a 16-state decoding scheme is shown in FIG. 3. In this case, one population of decoding probes will have half the number of detection molecules as compared to the other population of decoding probes. This allows differentiation between two different dinucleotide combinations which each comprise an A and a T, e.g., by quantifying the level of signal and not just the signal itself in the dinucleotide decoding scheme.

In this decoding scheme, examples of four different target sequence barcode sequences could be given by: target sequence 1: 1A(1/2)T, 1T(1/2)C, 1C(1/2)G, and 1A(1/2)A, target sequence 2: 1T(1/2)A, 1A(1/2)T, 1C(1/2)A, and 1A(1/2)G, target sequence 3: 1A(1/2)C, 1A(1/2)G, 1C(1/2)G, 1G(1/2)G, and target sequence 4: 1A(1/2)T, 1C(1/2)T, 1T(1/2)C, and 1A(1/2)G. Detection of the barcode sequence will include 4 rounds of detection. Each round of detection will comprise incubation of two populations of decoding probes. In the first round of detection, the first population of decoding probes will include combinations of the same sequence both with and without a 5′ dye modification, with the modified and unmodified sequences being present in equal proportions. The sequences that detect an T in the N1 position include 5′-Dye1-GTACGATGCA-3′ and 5′-GTACGATGCA-3′. The sequences that detect an A in the N1 position include 5′-Dye2-GTACGATGCT-3′ and 5′-GTACGATGCT-3′. The sequences that detect a G in the N1 position include 5′-Dye3-GTACGATGCC-3′, and 5′-GTACGATGCC-3′. The sequences that detect C in the N1 position include Dye4-GTACGATGCG-3′ and 5′-GTACGATGCG-3′. The second population of decoding probes will include 5′-p-ANNCGTATGC-3′Dye1, 5′-p-TNNCGTATGC-3′Dye2, 5′-p-CNNCGTATGC-3′Dye3, 5′-p-GNNCGTATGC-3′Dye4. The second round of decoding probes will include two populations. Again, the first population will include a combination of labeled and unlabeled probes. The first population of decoding probes will include the sequences that detect an T in the N3 position include 5′Dye1 ACGATGCNNA-3′, and 5′ACGATGCNNA-3′. The sequences that detect an A in the N3 position include 5′-Dye2-ACGATGCNNT-3′, and 5′-ACGATGCNNT-3′. The sequences that detect G in the N3 position include 5′-Dye3-ACGATGCNNC-3′ and 5′-ACGATGCNNC-3′. The sequences that detect C in the N3 position include 5′-Dye4-ACGATGCNNG-3′ and 5′-ACGATGCNNG-3′. The second population of decoding probes will include 5′-p-ACGTATGCGA-3′Dye1, 5′-p-TCGTATGCGA-3′Dye2, 5′-p-CCGTATGCGA-3′Dye3, 5′- and p-GCGTATGCGA-3′Dye4/.

In another decoding probe design, one nucleotide can be labelled by 2 dye molecules while the second nucleotide can be labelled by 1 dye molecule. In the first round of detection, the first population of decoding probes will include 5′-Dye1-GTACGA/T-Dye1/GCA-3′, 5′-Dye2-GTACGA/T-Dye2/GCT-3′, 5′-Dye3-GTACGA/T-Dye3/GCC-3′, 5′-Dye4-GTACGA/T-Dye4/GCG-3′, and the second population of decoding probes will include 5′-p-ANNCGTATGC-3′Dye1, 5′-p-TNNCGTATGC-3′Dye2, 5′-p-CNNCGTATGC-3′Dye3, 5′-p-GNNCGTATGC-3′Dye4. The second round of decoding probes will include two populations. The first population of decoding probes will include 5′-Dye1-ACGA/T-Dye1/GCNNA-3′, 5′-Dye2-ACGA/T-Dye2/GCNNT-3′, 5′-Dye3-ACGA/T-Dye3/GCNNC-3′, and 5′-Dye4-ACGA/T-Dye4/GCNNG-3′, and the second population of decoding probes will include 5′-p-ACGTATGCGA-3′Dye1, 5′-p-TCGTATGCGA-3′Dye2, 5′-p-CCGTATGCGA-3′Dye3, 5′- and p-GCGTATGCGA-3′Dye4/.

Each of Dye1, Dye2, Dye3, and Dye4 will be a fluorescent dye and be spectrally distinct. The first population of decoding probes in each round will include two dye molecules per detection molecule, with one dye molecule being incorporated internally. The second population of decoding probes in each round will include a 5′ phosphorylation modification to enable ligation between a member of the first population of decoding probes. Each additional round of detection will use similar populations of decoding probes. For each round of detection, probes from each of populations 1 and 2 will couple or bind to the barcode sequences within the sample corresponding to each of the target sequences. After binding, the bound probes will be ligated, and the non-ligated probes will be washed. The sample will be imaged using a fluorescence microscope. The ligated decoding probes from each round will be removed prior to addition of the decoding probes for the next cycle.

The signal(s) detected at each location during each cycle will be associated with a given dinucleotide sequence. Each nucleotide is uniquely associated with one of Dyes1-4 and its corresponding stoichiometry, and the combination of dye signals detected corresponds to one of the 16 possible dinucleotides. After completing four rounds of detection, the signals at each location corresponding to each of Dyes 1-4 at a given stoichiometry across all rounds will be analyzed. The analysis will involve correlating the combination of dye signals and levels at each location during each round to a dinucleotide identity and counting the frequency of each dinucleotide at each location. The total count of each dinucleotide at each location will be compared to the total dinucleotide count within each barcode sequence corresponding to each of the target sequences. Each location within the sample with a detectable signal will be identified as one of the target sequences based on the number of each dinucleotide detected.

### Example 4: Sequence Identification without Subsequence Order Information Using Bridge Oligonucleotides

In this example, 3876 target sequences will be identified using a 16-state decoding and 4 rounds of detection using bridging oligonucleotides to facilitate detection.

A sample comprising at least 3876 target sequences will be incubated with a plurality of binding moieties which each comprise a barcode sequence. The barcode sequences will include the following base sequence: 3′-CATGCTACG(N₁N₂)(N₃N₄)GCATACGCTTGATCG(N₅N₆)(N₇N₈)GATCAATACGG-5′, where each G, T, A, and C is representative of guanine (G), thymine (T), adenine (A), and cytosine (C), respectively. Each Nᵢ is representative of any one of A, C, T, or G at the given position. The identify of each target sequence will be represented by a quantity of each of 16 dinucleotides detected in any particular order. Each nucleotide of each dinucleotide will have a specific dye associated with it. The dye in each case may differ by either the dye molecule or the number of dye molecules associated with the detection event of the corresponding nucleotide. Details of the dinucleotide decoding scheme are the same as that described in Example 3. Detection of the barcode sequence will include 4 rounds of detection. Each round of detection will comprise incubation of two populations of decoding probes.

In the first round of detection, the first population of decoding probes will include 5′-Bridging-oligo1-GTACGATGCA-3′, 5′-Bridging-oligo2-GTACGATGCT-3′, 5′-Bridging-oligo3-GTACGATGCC-3′, and 5′-Bridging-oligo4-GTACGATGCG-3′, and the second population of decoding probes will include 5′-p-ANNCGTATGC-3′Bridging-oligo5/InvT/, 5′-p-TNNCGTATGC-3′Bridging-oligo6/InvT/, 5′-p-CNNCGTATGC-3′Bridging-oligo7/InvT/, and 5′-p-GNNCGTATGC-3′Bridging-oligo8/InvT/. The bridging oligo will comprise a nucleic acid sequence that is 5-20 nucleotides in length that is not complementary to the barcode sequence. Each of the bridging oligos will correspond to 3′ terminal nucleotide of the first population of decoding probes or 5′ terminal nucleotide of the second population of decoding probes. For each round of detection, probes from each of populations 1 and 2 will couple or bind to the barcode sequences within the sample corresponding to each of the target sequences. After binding, the bound probes with matched bases will be ligated, and un-ligated probes will be washed. Readout probes will be added to the sample for detecting signal associated with the ligated decoding probes. The first population of readout probes will include a complement or a reverse complement sequence to each of the four bridging oligos used corresponding to 3′ terminal nucleotide of the first population of decoding probes. Each of the four readout probes will be uniquely associated with a dye molecule. The dye molecules can be incorporated into the readout probes in different numbers such that a given dinucleotide identity may be a function of the sequence as well as the stoichiometry of dye molecules. The second population of readout probes will include a complement or a reverse complement sequence to each of the four bridging oligos used corresponding to 5′ terminal nucleotide of the second population of decoding probes. Similarly, each of the four readout probes will be uniquely associated with a dye molecule. The dye molecules can be incorporated into the readout probes in different numbers such that a given dinucleotide identity may be a function of the sequence as well as the stoichiometry of dye molecules. The sample will be imaged. The readout probes and the decoding probes will be removed, and a second round of decoding and readout will be performed in a similar manner. A total of four rounds of detection will be performed.

The signal(s) detected at each location during each cycle will be associated with a given dinucleotide sequence. Each nucleotide is uniquely associated with one of Dyes1-4 and its corresponding stoichiometry, and the combination of dye signals detected corresponds to one of the 16 possible dinucleotides. After completing four rounds of detection, the signals at each location corresponding to each of Dyes 1-4 at a given stoichiometry across all rounds will be analyzed. The analysis will involve correlating the combination of dye signals and levels at each location during each round to a dinucleotide identity and counting the frequency of each dinucleotide at each location. The total count of each dinucleotide at each location will be compared to the total dinucleotide count within each barcode sequence corresponding to each of the target sequences. Each location within the sample with a detectable signal will be identified as one of the target sequences based on the number of each dinucleotide detected.

### Example 5: Sequence Identification without Subsequence Order Information

In this example, 50,625 target sequences (15⁴) will be identified using a 15-state decoding and 4 rounds of detection without the need of arranging the detection rounds in specific temporal order.

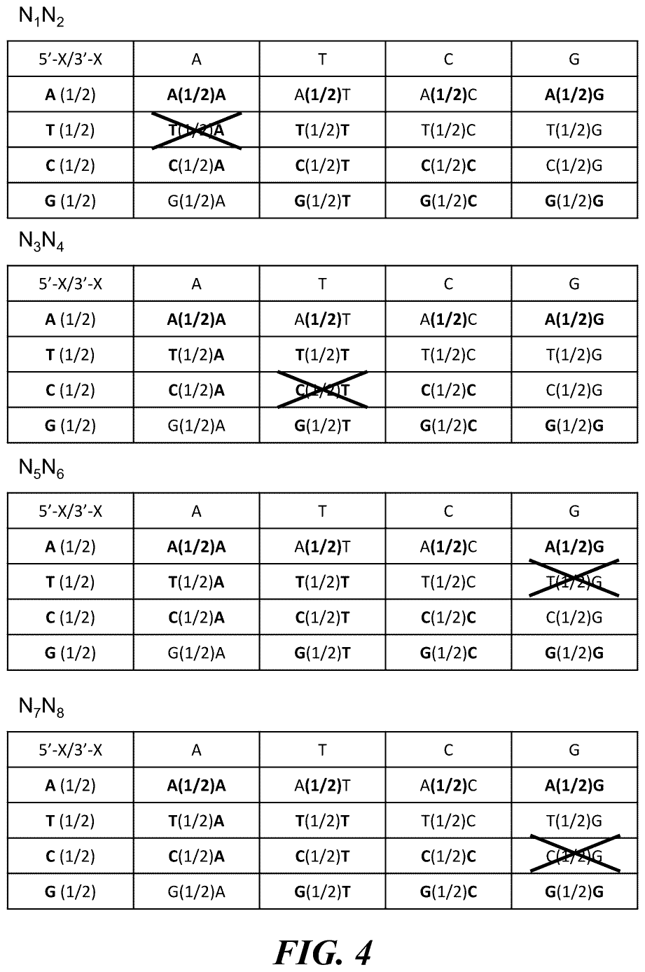

A sample comprising at least 50,625 target sequences will be incubated with a plurality of binding moieties which each comprise a barcode sequence. The barcode sequences will include the base following sequence: 3′-CATGCTACG(N₁N₂)(N₃N₄)GCATACGCTTGATCG(N₅N₆)(N₇N₈)GATCAATACGG-5′, where each G, T, A, and C is representative of guanine (G), thymine (T), adenine (A), and cytosine (C), respectively. Each Nᵢ is representative of any one of A, C, T, or G at the given position with the except of one specific di-nucleotide combination in any given position (FIG. 4). The information of the non-existing di-nucleotide in any given round will give the information of the specific position of N₁N₂, N₃N₄, N₅N₆, or N₇N₈. The identify of each target sequence will be represented by a quantity of each of 15 dinucleotides detected in a particular order. Details of the dinucleotide decoding scheme are the same as that described in Example 3 with the exception that for each dinucleotide position of the barcode sequence, one dinucleotide will be omitted as an option. The combinations for an example code scheme are shown in FIG. 4.

The detection probes will be the same as those described in Example 3. Detection probes will be added to the sample, the sample imaged, and detection probes removed during each cycle. Four rounds of detection will be performed without the requirement of specifying the temporal order.

The signal(s) detected at each location during each cycle will be associated with a given dinucleotide sequence. Each nucleotide is uniquely associated with one of Dyes1-4 and its corresponding stoichiometry, and the combination of dye signals detected corresponds to one of the 16 possible dinucleotides. After completing four rounds of detection, the signals at each location corresponding to each of Dyes 1-4 at a given stoichiometry across all rounds will be analyzed. In addition to this information, combinations of dinucleotides detected at each cycle will be assessed and compared to the 16 possible configurations. Each cycle will involve the omission of a particular dinucleotide, and the omission will correspond to a given cycle based on the configuration shown in FIG. 4. The analysis will involve correlating the combination of dye signals and levels at each location during each round to a dinucleotide identity, and correlating the collection of all detected dinucleotide identities in any decoding round to the round-specific omission of the specific dinucleotides to identify the specific dinucleotide position out of N₁N₂, N₃N₄, N₅N₆, or N₇N₈ this decoding round is probing based on the configuration shown in FIG. 4, and subsequently identify each dinucleotide identity within the barcode. The identity of all four dinucleotides at each location will be compared to the four dinucleotide identities at each position within each barcode sequence corresponding to each of the target sequences. Each location within the sample with a detectable signal will be identified as one of the target sequences based on the identity of all four dinucleotide detected.

### Example 6:8 Gene Decoding Using Dinucleotide Detection

In this example, 8 mRNA sequences were identified using a 10-state decoding and 4 rounds of detection without the need of arranging the detection rounds in specific temporal order.

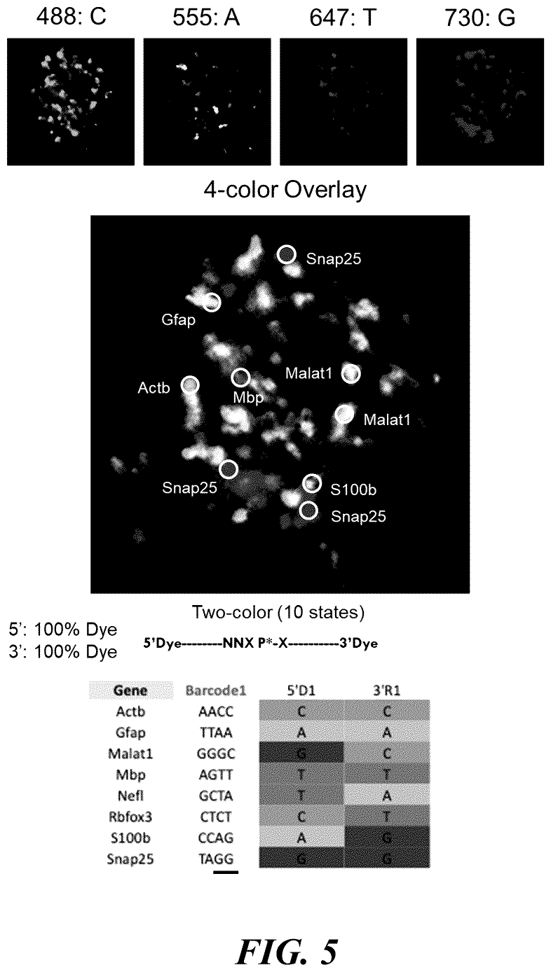

A 20 μm thick fresh frozen mouse brain tissue section was sliced onto a coverslip. The sample was fixed with formaldehyde and incubated with a set of padlock probe sequences. Each of the padlock probe sequences of the set of padlock probe sequences included a target binding sequence that recognized one of eight transcripts: ActB, Gfap, Malat1, Mpb, NefI, Rbfox3, S100b, and Snap25A. An additional oligonucleotide sequence was added corresponding to each padlock probe sequence that included a target-binding sequence adjacent to the target binding sequence on the padlock probe and a padlock probe binding sequence that couples or binds to 5′ and 3′ end of the corresponding padlock probe sequence. Upon binding, the padlock probe sequences, and additional oligonucleotides sequences formed complexes on the mRNA target sequences. A ligase was added to the sample to form a circular oligonucleotide and phi29 polymerase was added to initiate a rolling circle amplification to form an amplification product at each location on each mRNA bound by the padlock probe sequences. Each padlock probe sequence comprised a barcode sequence according to the list depicted by in FIG. 5. A dinucleotide combination of each barcode was detected by binding two different sets of detection probes according to the structure depicted in FIG. 5. The X position was given by each of the four nucleotides: A, T, C, and G, where a given X nucleotide was associated with a unique dye according to the following scheme: AlexaFluor488=C(488 laser channel), AlexaFluor546=A (555 laser channel), AlexaFluor647=T (647 laser channel), and AlexaFluor750=G (750 laser channel). The N was given by every permutation of A, T, C, and G. After binding the detection probes, a ligase was added to create a continuous probe comprising a dye molecule at both 3′ and 5′ ends. The signal associated with the detection probes was imaged using a fluorescence microscope. A representative single channel image of each of the four dye channels for a given region of interest is shown in the top pane of FIG. 5. After detection, detection probes were removed using a high percentage formamide solution. A second round of detection was performed in a similar manner. A 4-color overlay image showing the signal across all four channels for a given cycle of detection. The overlay image is annotated with example instances of each of the given mRNA targets based on the signal across both cycles and four channels.

### Example 7: Detection of Dinucleotides Associated with Barcode Sequences Associated with 250 mRNA Targets

In this example, dinucleotides associated with the barcode sequences of 250 mRNA sequences were identified using multiple different decoding schemes.

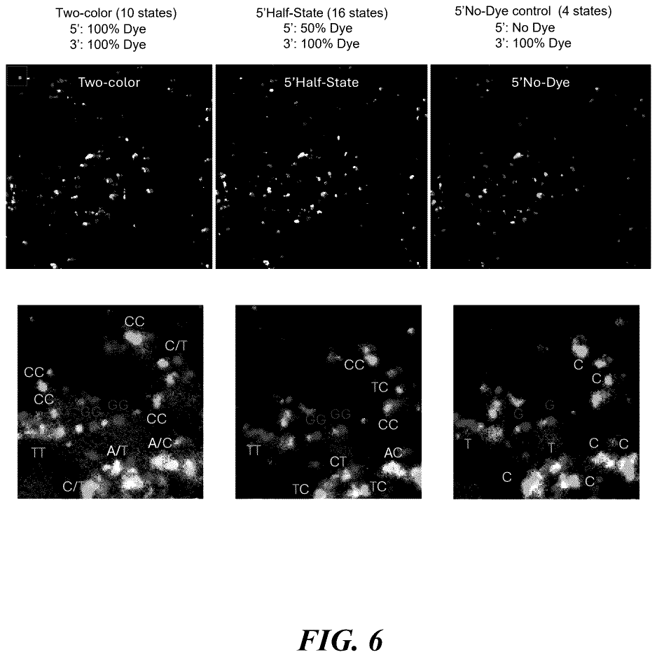

A 20 μm thick fresh frozen mouse brain tissue section was sliced onto a coverslip. The sample was fixed with formaldehyde and incubated with a set of padlock probe sequences. Each of the padlock probe sequences of the set of padlock probe sequences included a target binding sequence that recognized one of 250 transcripts. An additional oligonucleotide sequence was added corresponding to each padlock probe sequence that included a target-binding sequence adjacent to the target binding sequence on the padlock probe and a padlock probe binding sequence that couples or binds to 5′ and 3′ end of the corresponding padlock probe sequence. Upon binding, the padlock probe sequences, and additional oligonucleotides sequences formed complexes on the mRNA target sequences. A ligase was added to the sample to form a circular oligonucleotide and phi29 polymerase was added to initiate a rolling circle amplification to form an amplification product at each location on each mRNA bound by the padlock probe sequences. Each padlock probe sequence comprised a barcode sequence that was uniquely associated with one of the mRNA targets. Detection of dinucleotide combinations associated with the barcode sequences was performed by adding two sets of detection probes to the sample. In the first cycle, detection probes were added based on a 10-state detection scheme according to FIG. 2. A representative image of the colocalized spots is shown in the image on the left of FIG. 6 with annotated combinations of nucleotides indicated. In a second cycle, detection probes were added based on a 16-state detection scheme, as outlined in FIG. 3. A representative image of the colocalized spots is shown in the image in the middle of FIG. 6 with annotated combinations of nucleotides indicated. In a third cycle, detection probes were added based on a 4-state detection scheme, where only one dye was associated with the ligated detection probe product. A representative image of the singled nucleotide annotated spots are shown in the image on the right of FIG. 6. While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Example 8: Detection of Dinucleotides Associated with Barcode Sequences Associated with 9 mRNA Targets

In this example, dinucleotides associated with the barcode sequences of mRNA sequences were identified using multiple different decoding schemes.

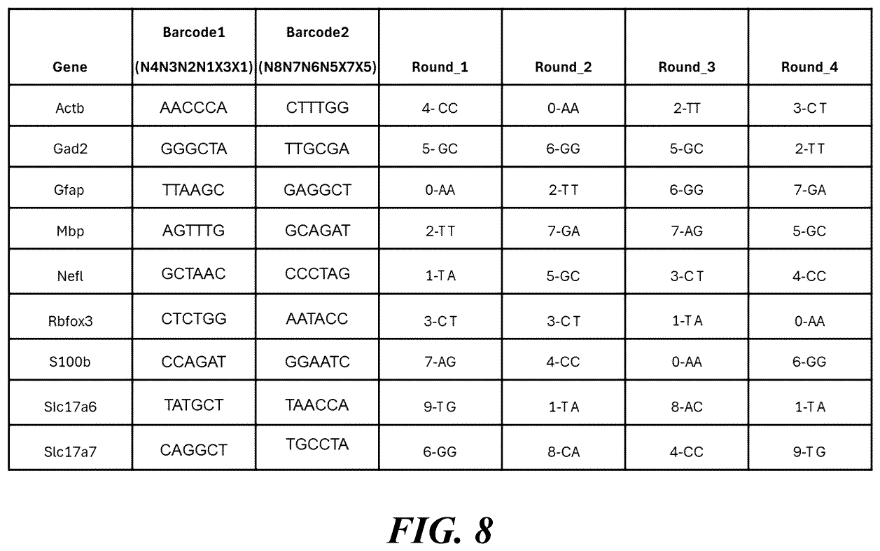

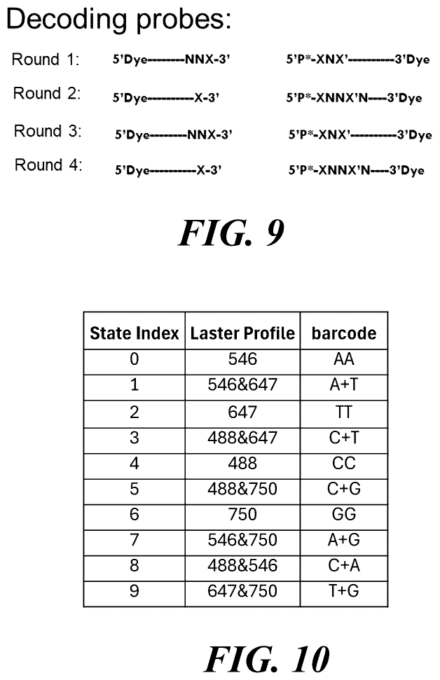

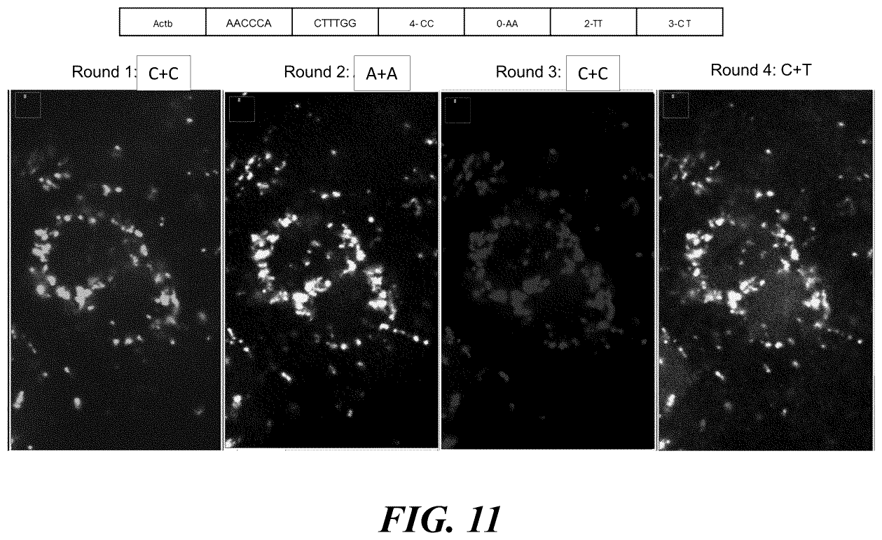

A 20 μm thick fresh frozen mouse brain tissue section was sliced onto a coverslip. The sample was fixed with formaldehyde and incubated with a set of padlock probe sequences. Each of the padlock probe sequences of the set of padlock probe sequences included a target binding sequence that recognized one of 9 transcripts. An additional oligonucleotide sequence was added corresponding to each padlock probe sequence that included a target-binding sequence adjacent to the target binding sequence on the padlock probe and a padlock probe binding sequence that couples or binds to 5′ and 3′ end of the corresponding padlock probe sequence. Upon binding, the padlock probe sequences, and additional oligonucleotides sequences formed complexes on the mRNA target sequences. A ligase was added to the sample to form a circular oligonucleotide and phi29 polymerase was added to initiate a rolling circle amplification to form an amplification product at each location on each mRNA bound by the padlock probe sequences. Each padlock probe sequence comprised two barcode sequences that were uniquely associated with one of the mRNA targets. The details of the barcode sequences are shown in FIG. 8. For each barcode sequence shown in FIG. 8, the first four nucleotides were used to detect the mRNA sequence, whereas the last two nucleotides were added to reduce mis-ligation of a 3′ end of the detection probe with a 5′ end of another detection probe. For example, a mis-ligation for 3′ end of the detection probe may result in a two-nucleotide mismatch. The decoding probes (detection probes) used to detect dinucleotide combinations within the barcode sequences of FIG. 8 are shown in FIG. 9. Four rounds of detection were performed. The ‘N′’s listed in the decoding probes of FIG. 9 indicate that every combination of nucleotides was included at that nucleotide position (e.g. detection probes that include an A, C, G, and T were included). The ‘X′’s listed in the decoding probes of FIG. 9 correspond to the nucleotides associated with each barcode sequence. The P* in FIG. 9 indicates a 5′ phosphorylation group. Each decoding probe in FIG. 9 includes a dye. The laser profile of the different dyes used is shown in FIG. 10. A detection probe with a terminal A had a dye with an absorption profile peak at 546 nm. A detection probe with a terminal T had a dye with an absorption profile peak at 647 nm. A detection probe with a terminal G had a dye with an absorption profile peak at 750 nm. A detection probe with a terminal C had a dye with an absorption profile peak at 488 nm. For example, in Barcode 1 for Actb, the first round of detection detected a ‘CC’ dinucleotide. Thus, the only signal detected was 488. As another example, the first round of detection for detecting the Gad2 barcode detected a ‘GC’ dinucleotide. The combination of 488 nm signal and 750 nm signal indicated the presence of that dinucleotide.

Representative image data for detecting ActB mRNA is shown in FIG. 11. Here, images of the same region of interest across four rounds of detection are shown. The first dinucleotide detected for this barcode is ‘CC’ (the middle dinucleotide of the first barcode sequence: AACCCA). Based on the detection scheme shown in FIG. 10, the only signal associated with ‘CC’ is 488 nm. The first image on the left of FIG. 11 shows the signal for the 488 nm channel. The second dinucleotide detected for this barcode is ‘AA’ (the first dinucleotide of the first barcode sequence: AACCCA). Based on the detection scheme shown in FIG. 10, the only signal associated with ‘AA’ is 546 nm. The second image on the left of FIG. 11 shows the signal for the 546 nm channel. The third dinucleotide detected for this barcode is ‘TT’ (the second dinucleotide of the second barcode sequence: CTTTGG). Based on the detection scheme shown in FIG. 10, the only signal associated with ‘TT’ is 647 nm. The third image on the left of FIG. 11 shows the signal for the 546 nm channel. The fourth dinucleotide detected for this barcode is ‘CT’ (the first dinucleotide of the second barcode sequence: CTTTGG). Based on the detection scheme shown in FIG. 10, the two signals associated with ‘CT’ are 546 nm and 647 nm. The fourth image on the left of FIG. 11 shows the overlay image of signal for the 546 nm channel and signal for the 647 nm channel. As shown, the signal pattern across the four images shows overlap and the locations of the ActB mRNA sequences within the sample.

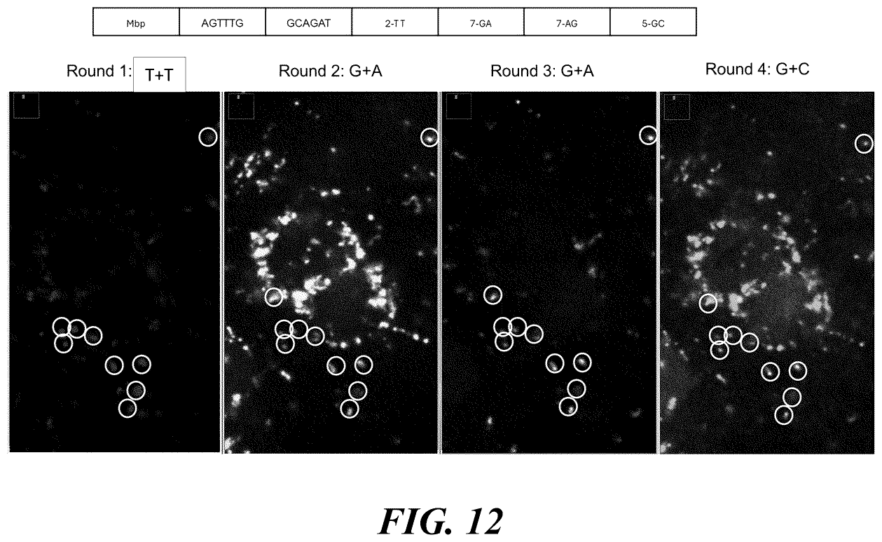

Representative image data for detecting Mbp mRNA is shown in FIG. 12. Here, images of the same region of interest across four rounds of detection are shown. The first dinucleotide detected for this barcode is ‘TT’ (the middle dinucleotide of the first barcode sequence: AGTTTG). Based on the detection scheme shown in FIG. 10, the only signal associated with ‘TT’ is 647 nm. The first image on the left of FIG. 12 shows the signal for the 647 nm channel. The second dinucleotide detected for this barcode is ‘GA’ (the first dinucleotide of the first barcode sequence: AGTTTG). Based on the detection scheme shown in FIG. 10, the signal associated with ‘GA’ is 750 nm and 546 nm. The second image on the left of FIG. 12 shows the overlay image of the signal for the 750 nm channel and the 546 nm channel. The third dinucleotide detected for this barcode is ‘AG’ (the second dinucleotide of the second barcode sequence: GCAGAT). Based on the detection scheme shown in FIG. 10, the signal associated with ‘AG’ is 546 nm and 750 nm. The third image on the left of FIG. 12 shows the overlay image of the signal for the 546 nm channel and the 750 nm channel. The fourth dinucleotide detected for this barcode is ‘GC’ (the first dinucleotide of the second barcode sequence: GCAGAT). Based on the detection scheme shown in FIG. 10, the two signals associated with ‘GC’ are 750 nm and 488 nm. The fourth image on the left of FIG. 12 shows the overlay image of signal for the 750 nm channel and signal for the 488 nm channel. As shown, the signal pattern across the four images shows overlap and the locations of the Mbp mRNA sequences within the sample.

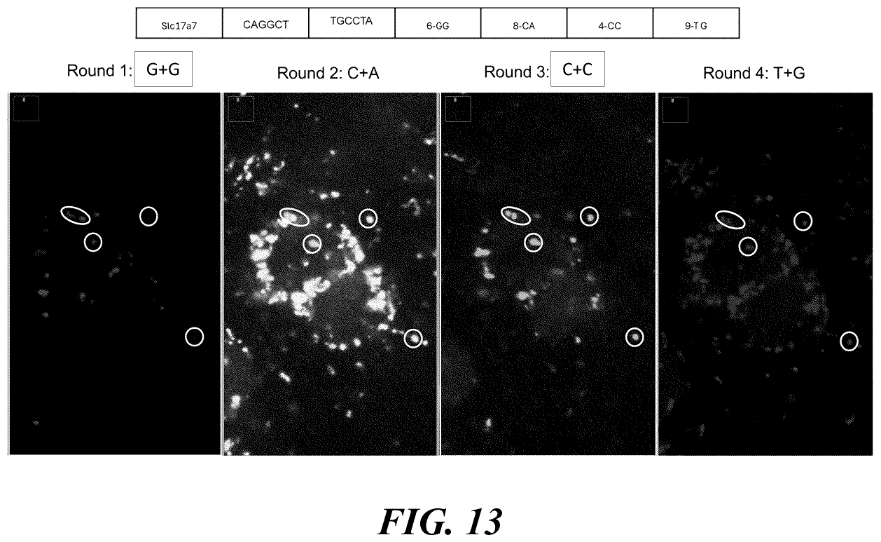

Representative image data for detecting Slc17a7 mRNA is shown in FIG. 13. Here, images of the same region of interest across four rounds of detection are shown. The first dinucleotide detected for this barcode is ‘GG’ (the middle dinucleotide of the first barcode sequence: CAGGCT). Based on the detection scheme shown in FIG. 10, the only signal associated with ‘GG’ is 750 nm. The first image on the left of FIG. 13 shows the signal for the 750 nm channel. The second dinucleotide detected for this barcode is ‘CA’ (the first dinucleotide of the first barcode sequence: CAGGCT). Based on the detection scheme shown in FIG. 10, the signal associated with ‘CA’ is 488 nm and 546 nm. The second image on the left of FIG. 13 shows the overlay image of the signal for the 488 nm channel and the 546 nm channel. The third dinucleotide detected for this barcode is ‘CC’ (the second dinucleotide of the second barcode sequence: TGCCTA). Based on the detection scheme shown in FIG. 10, the only signal associated with ‘CC’ is 488 nm. The third image on the left of FIG. 13 shows the signal for the 488 nm channel. The fourth dinucleotide detected for this barcode is ‘TG’ (the first dinucleotide of the second barcode sequence: TGCCTA). Based on the detection scheme shown in FIG. 10, the two signals associated with ‘TG’ are 647 nm and 750 nm. The fourth image on the left of FIG. 13 shows the overlay image of signal for the 647 nm channel and signal for the 750 nm channel. As shown, the signal pattern across the four images shows overlap and the locations of the Slc17a7 mRNA sequences within the sample.

1. A method for identifying an analyte in a sample, comprising:
(a) contacting an analyte with a binding moiety, wherein said binding moiety comprises (i) a probe that couples to said analyte and (ii) a sequence, wherein said sequence comprises a plurality of subsequences;
(b) bringing said sample in contact with a first detection probe and a second detection probe, wherein:
(i) said first detection probe couples to a first subsequence of said sequence or derivative thereof, and wherein said first detection probe comprises a first detection moiety;
(ii) said second detection probe couples to a second subsequence of said sequence or derivative thereof, and wherein said second detection probe comprises a second detection moiety; and
(iii) said first subsequence and said second subsequence are adjacent to each other;
(c) detecting said first detection moiety and said second detection moiety to identify at least said first subsequence and said second subsequence; and
(d) using at least said first subsequence and said second subsequence identified in (c) to identify said analyte.

2. The method of claim 1, wherein said sample is a tissue.

3. The method of claim 1, wherein said sample is embedded in a hydrogel.

4. The method of claim 1, wherein said analyte comprises a nucleic acid.

5. The method of claim 4, wherein said nucleic acid is ribonucleic acid.

6. The method of claim 1, wherein said probe that couples to said analyte comprises a nucleic acid.

7. The method of claim 1, wherein a subsequence of said plurality of subsequences comprises a nucleic acid.

8. The method of claim 1, wherein said plurality of subsequences comprises subsequences of the same length.

9. The method of claim 1, wherein said plurality of subsequences comprises subsequences of different lengths.

10. The method of claim 1, wherein said first detection probe comprises a nucleic acid.

11. The method of claim 1, wherein said second detection probe comprises a nucleic acid.

12. The method of claim 1, wherein said first detection moiety comprises a fluorescent dye.

13. The method of claim 1, wherein said second detection moiety comprises a fluorescent dye.

14. The method of claim 1, wherein (c) comprises imaging said analyte.

15. The method of claim 14, wherein said imaging comprises using a microscope.

16. The method of claim 1, further comprising amplifying said sequence in (a) to generate an amplified sequence, wherein said amplified sequence comprises multiple copies of said sequence or derivative thereof.

17. The method of claim 1, further comprising ligating said first detection probe and said second detection probe in (b).

18. The method of claim 17, wherein said ligating comprises using a ligase.

19. The method of claim 1, wherein (c) further comprises detecting a ratio of said first detection moiety and said second detection moiety.

20. The method of claim 19, further comprising using said ratio to identify said analyte.
